(19) United States
(12) Patent Application Publication
(10) Pub. No.: US 2024/0016762 A1
(43) Pub. Date: Jan. 18, 2024

(54) DUAL ANALGESIC/ANTI-INFLAMMATORY COMPOSITIONS COMPRISING CB2 RECEPTOR AGONISTS, COMBINATIONS, AND METHODS OF USE THEREOF

(71) Applicant: Altus Formulation lnc., Mirabel (CA)

(72) Inventors: Damon SMITH, Montreal (CA); Bradut MITRASCA, Laval (CA); Piotr KUJAWA, Montreal (CA); Alexander CUNNINGHAM, Dollard-des-Ormeaux (CA); Anna-Maria SZCZESNIAK, Fergusons Cove (CA); Melanie KELLY, Fergusons Cove (CA); Robert ZALEGA, Montreal (CA); Fouzia SOULHI, Pierrefonds (CA); Sylvain CHEMTOB, Cote St Luc (CA); Samy OMRI, Montreal (CA)

(21) Appl. No.: 18/042,145

(22) PCT Filed: Aug. 19, 2021
(86) PCT No.: PCT/CA2021/051152

§ 371 Date: Feb. 17, 2023

Related U.S. Application Data
(60) Provisional application No. 63/068,737, filed on Aug. 21, 2020.
(60) Provisional application No. 63/087,038, filed on Oct. 2, 2020.
(60) Provisional application No. 63/191,107, filed on May 20, 2021.

(51) Int. Cl. A61K 31/09; A61K 47/34; A61K 9/107; A61K 9/19; A61K 31/635; A61K 9/06; A61K 47/38; A61K 47/26; A61K 47/18; A61K 47/32; A61K 9/20; A61K 9/28; A61K 9/48

(52) U.S. Cl. CPC A61K 31/09; A61K 47/34; A61K 9/1075; A61K 9/19; A61K 31/635; A61K 9/06; A61K 47/38; A61K 47/26; A61K 47/186; A61K 47/32; A61K 9/205; A61K 9/2013; A61K 9/2009; A61K 9/28; A61K 9/2054; A61K 9/2059; A61K 9/4808; A61K 9/4866; A61K 9/485; A61K 9/4858

(57) ABSTRACT

Herein is described a pharmaceutical composition for agonizing CB2 receptor activity, medical kits, therapeutic applications thereof, and methods of making and using such compositions. The composition comprises a combination of: —a first compound of Formula I: (E1) or a pharmaceutically acceptable salt thereof; and —a second compound of Formula II: (E2) or a pharmaceutically acceptable salt thereof.

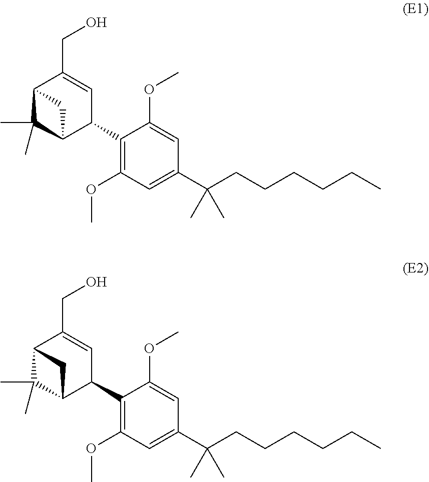

### RELATED APPLICATIONS

This application claims the benefit of priority of U.S. Provisional Application No. 63/068,737 entitled “DUAL ANALGESIC/ANTI-INFLAMMATORY COMPOSITIONS, COMBINATIONS, AND METHODS OF USE THEREOF” and filed Aug. 21, 2020; of U.S. Provisional Application No. 63/087,038 entitled “DUAL ANALGESIC/ANTI-INFLAMMATORY COMPOSITIONS, COMBINATIONS, AND METHODS OF USE THEREOF” and filed Oct. 2, 2020; and of U.S. Provisional Application No. 63/191,107 entitled “DUAL ANALGESIC/ANTI-INFLAMMATORY COMPOSITIONS, COMBINATIONS, AND METHODS OF USE THEREOF” and filed May 20, 2021, the foregoing three applications being incorporated by reference in their respective entireties.

### FIELD OF THE INVENTION

The invention provides, in part, pharmaceutical compositions for agonizing cannabinoid type 2 (CB₂) receptor activity in a subject, medical kits, and methods of making and using such compositions.

### BACKGROUND

When a subject is injured, for example via physical trauma resulting from an accident or surgery, various response mechanisms are initiated to protect the subject from further injury and initiate the healing processes. For example, immediately after an injury occurs, fast acting peripheral nerve fibers (“nociceptors”) are activated to trigger muscle recoil and the reinforcing sensation of piercing pain thereby reducing the risk of further injury (Basbaum, et al. (2009) CELL, 139(2): 267-84). The injury also triggers local inflammation.

Inflammation is a first-line mammalian response to injury and is mediated, predominantly by white cells of the innate immune system (Janeway, et al. (2001) IMMUNOBIOLOGY: THE IMMUNE SYSTEM IN HEALTH AND DISEASE. 5th edition. New York: Garland Science). These cells, when activated by injury damaged tissue, transform rapidly to amplify the initial, acute response in several ways (Krystel-Whittemore, et al. (2016) FRONT. IMMUNOL., 6: 620; Zhang, et al. (2007) INTERNATIONAL ANESTHESIOLOGY CLINIC, 45(2): 27-37); Vance, et al. (2009) CELL HOST & MICROBE, 6(1): 10-21). For example, the expression of inducible cyclooxygenase 2 (COX-2) is upregulated causing rapid increases in prostanoids such as prostaglandin E2 (PGE2) and thromboxane (Cadieux, et al. (2005) J. CELL SCIENCE, 118(Pt 7): 1437-47). These early phase molecules increase the pain sensation induced by local nociceptors (Zeilhofer, et al. (2007) BIOCHEMICAL PHARMACOL., 73: 165-174), activate remote nociceptors (Funk (2001) SCIENCE, 294(5548): 1871-5; Reinold et al. (2005) J. CLIN. INVEST., 115: 673-679; Lehnardt et al. (2003) PROC. NATL. ACAD. SCI. USA, 100: 8514-8519) and promote arterial dilation and enhanced tissue permeability (Ricciotti, et al. (2011) ARTERIOSCLER. THROMB. VASC. BIOL., 31(5): 986-1000). These latter effects facilitate immune cell access to the area and increase local blood flow creating the classic signs of inflammation such as visual redness, swelling, and raised temperature. Prostanoids also augment the actions of other immune-mediators such as chemokines.

Chemokines are chemoattractants that induce circulating immune cells to migrate to the inflamed area (Krystel-Whittemore (2016) supra; Zhang, et al. (2007) supra; Vance (2009) supra). Due to their high numbers in circulation, neutrophils often are the first cells recruited to a site of injury and their increased presence, along with macrophages, and T and B lymphocytes, magnifies the immune response heightening the pain sensation and increasing swelling. Together these effects further mitigate additional injury to the site.

New immune cells accumulating at the site of inflammation also increase local cytokine concentrations particularly those of IL-1β and TNF∞, which act synergistically to further intensify vasodilation and tissue permeability, and upregulate adhesion molecules to retain the newly arrived immune cells.

Along with intensifying inflammation, cytokines also increase the pain response and the sensation of pain. For example, IL-1β has been shown to be a potent mechanical and thermal hyperalgesic agent in peripheral tissues (Ferreira S H, et al. (1988) NATURE, 334: 698-700; Zelenka, et al. (2005) PAIN, 116(3): 257-263.) while, conversely TNF∞ causes central, CNS mediated algesia (Hess, et al. (2011) PROC. NATL. ACAD. SCI., 108(9): 3731-6). Bradykinin, another early phase cytokine, generates a similarly painful sensation, which collectively with the other cytokines results in acute pain.

Acute pain is temporary and typically diminishes during the healing process. However, along with mediating inflammation, activated immune cells also release a range of toxic molecules to destroy pathogens infecting the injured tissue. These include reactive oxygen species (such as the superoxide anion, hydrogen peroxide, hydroxyl radicals and hypochlorous acid) (Teng, et al. (2017) J. IMMUNOL RES., 2017: 9671604) and destructive enzymes such as collagenases and myeloperoxidases, all of which come into contact with host tissues (Brinkmann, et al. (2004) SCIENCE, 303(5663): 1532-1535; Wang (2018) CELL TISSUE RES., 371(3): 531-539).

Should the source of infection not be eliminated, the injury not heal, or the cause of immune cell activation continue (as occurs in autoimmune diseases), host tissues can experience significant damage from the continued response. In these situations, acute inflammation may transform into chronic inflammation and long-term damage to both local, and distant, tissues can occur (Garn, et al. (2016) J. ALLERGY AND CLIN. IMMUNOL., 138(1): 47-56). Acute pain will also transform into chronic pain, which through nerve remodeling becomes difficult to treat (Voscopoulos, et al. (2010) BR. J. ANAESTH, 105(S1): 69-85).

Thus, while acute inflammation and the resulting pain are beneficial (yet unpleasant) reactions to minimize host damage and promote healing, chronic inflammation is a disease state that causes host-tissue damage and long-term pain. Although formerly thought of as an isolated condition, there is now a growing body of evidence suggesting that chronic inflammation underlies a range of debilitating diseases, including certain cardiovascular diseases, diseases of the eye, multiple sclerosis, and cancer.

Early and effective control of inflammation is therefore important in controlling acute pain, avoiding chronic pain and preventing recurrent tissue damage.

It has become apparent that certain agents with the ability to inhibit inflammation can also act as analgesics directly by inhibiting nociceptor sensitization and cytokine release, and indirectly via inhibition of the allodynic effects of swelling. Non-Steroidal Anti-Inflammatory Drugs (NSAIDs) and Corticosterids (Steroids) are two such classes of anti-inflammatory agent.

A number of NSAID medicines are available for use as analgesics including, for example, ketorolac (Toradol®; Roche), diclofenac (Voltaren®; Novartis) and ibuprofen (Nurofen®; Reckitt Benckiser). As a class, these drugs bind to and inhibit both constitutive COX-1 enzymes and inducible COX-2 enzymes found throughout the body thereby decreasing the prostanoid production noted above. NSAIDs are used systemically for acute conditions such as postoperative somatic pain and for chronic ailments such as osteoarthritis and rheumatoid arthritis. A number of NSAIDs are also approved for local use for example in the eye, for the treatment of pain associated with acute conditions such as post-surgical pain arising from cataract removal (Hoffman, et al. (2016) J. CATARACT REFRACT. SURG., 42(9): 1368-1379), Photorefractive Keratectomy (PRK) (Razmju, et al. (2012) INT. J. PREV. MED., 3(Supp 1): S199-S206)), corneal cross linking (CXL) (Sameen, et al. (2017) PAK. J. MED. SCI., 33(5): 1101-1105), and chronic conditions such as dry-eye syndrome (Colligris, et al. (2014) SAUDI J. OPHTHALMOL. 28(1): 19-30).

Although generally effective, the broad specificity of NSAIDS, particularly their inhibition of constitutive COX-1, can generate serious side-effects and, as a class, they display warnings for cardiovascular toxicity (including heart attack and stroke) and bleeding risk. Because of these dangers, certain NSAIDs, for example, Toradol® and Voltaren® are contraindicated for many subjects (especially elderly subjects) and for treating indications where bleeding can occur, including during cardiac surgery. Employing NSAIDs to treat local pain and inflammation may also be problematic because, as a class they are linked with delays in wound healing which increases the duration of pain and the risk of infection (Iwamoto, et al. (2017) NATURE SCIENTIFIC REPORTS, 7: 13267). The use of NSAIDs in the eye has also been associated with corneal melting, a serious condition in which the corneal epithelium and underlying stroma are lost leading to perforation and blindness (Flach (2001) TRANS. AM. OPHTHALMOL. SOC., 99: 205-212).

COX-2 inhibitors are a sub-set of traditional NSAIDs that inhibit predominantly the inducible form of COX-2 generated at an inflammation site, and decrease the prostanoid levels at that site. COX-2 drugs may therefore avoid the systemic side-effects of COX-1 inhibition seen with traditional NSAIDs. Evidence of this comes from a European study (Arfè, et al. (2016) BRIT. MED. J., 354: i4857) where non-specific NSAID drugs taken for their analgesic effects, including ketorolac, naproxen and ibuprofen, increased the risk of heart failure in these patients while COX-2 specific inhibitors given to the same population did not pose such a risk. Apart from their benefits during systemic use, COX-2 inhibitors have also shown advantages over COX-1 inhibitors when delivered locally. For example, celecoxib (Celebrex®; Pfizer) a potent inhibitor of COX-2, when administered directly to the eye has been shown to mitigate the debilitating effects of age-related macular degeneration and diabetic retinopathy, two leading causes of blindness (Kompella, et al. (2010) EXPERT OPIN. DRUG DELIV., 7(5): 631-645). COX-1 inhibition was unable to achieve these benefits. Despite these advantages however, COX-2 drugs as a class display warnings for severe side effects including cardiovascular and gastrointestinal toxicity (see, e.g., Highlights of Prescribing information for CELEBREX® June 2018 revision available on the www at accessdata.fda.gov/drugsatfda_docs/label/2018/020998s0501bl.pdf).

Corticosteroids (steroids) are broad spectrum anti-inflammatory drugs that mimic hormones generated by the adrenal glands. The value of steroids is their general applicability to a wide range of indications and ability to enter numerous cell types (Oren, et al. (2004) BIOPHYS. J. 87(2): 768-79). Upon entry into a cell, steroids bind rapidly to glucocorticoid receptors (GR) in the cytoplasm thereby initiating two complimentary anti-inflammatory pathways; at low concentrations steroids act indirectly via gene coactivators such as NF-κB to inhibit production of pro-inflammatory cytokines, chemokines, and adhesion molecules (Hua (2013) FRONT PHARMACOL., 4: 127); at higher concentrations steroids also interact directly with DNA to induce the production of anti-inflammatory molecules such as lipocortin 1, IκB-α and IL-10 (Dostert, et al. (2004) CURR. PHARM. DES., 10: 2807-2816; Couper, et al. (2008) J. IMMUNOL., 180(9): 5771-5777). Production of these molecules helps to silence any ongoing inflammatory processes.

Due to these profound immunomodulatory effects, steroids are today amongst the most widely prescribed drugs in the world and their use continues to increase (see, e.g., Dennison, et al. (1998) B.M.J., 316(7134): 789-90). The analgesic effects of steroids are limited however. For example, in a meta-analysis of 14 controlled-trials to study corticosteroid mediated postoperative analgesia, corticosteroids achieved only very minor reductions in pain when given orally and no benefits when given locally (Mohammad, et al., (2017) SYSTEMATIC REV., 6(1): 92). The evidence for pain control following ophthalmic surgery is also limited despite their wide use.

The ubiquitous cellular uptake of steroids and their pleiotropic activity are benefits for broad efficacy. However, the lack of specificity can result in extensive off-target binding and the resulting side effects associated with steroid use (e.g., hypertension, weight gain, diabetes, cataracts, glaucoma, venous thromboembolism, and bone fracture) can be extensive and serious.

NSAID, COX-2 and steroid drugs provide wide-ranging and significant benefits for the treatment of inflammation while NSAID and COX-2 drugs have an ability, not observed with steroids, to control acute pain. Despite this advantage however, the analgesic effect of NSAIDs and COX-2 drugs is also limited as both display a saturation, or ceiling effect, after which higher administered doses generate no greater analgesia, though side effects continue to rise (Motov, et al. (2017) ANN. EMERG. MED., 70(2): 177-184). As a result, their value is diminished for those in severe or extreme pain. This limitation cannot be overcome by combining NSAIDs and/or COX-2 drugs as their common mechanism of action leads only to cumulative side-effects not increased pain relief. As a result, physicians generally do not rely on NSAIDs and COX-2 drugs alone to treat those with extreme pain.

Similarly, corticosteroid medications all share a common mechanism of action such that they too cannot be combined for greater effect as severe and long-lasting side-effects may result. Their lack of analgesic effect also dictates that a combination of steroids with NSAIDs or COX-2 drugs is unlikely to be of benefit to patients with uncontrolled pain, especially in the acute phase where the delayed onset time for steroids is a concern (Williams (2018) CLIN. PHARMACOL. CORTICOSTEROIDS RESP. CARE, 63(6): 655-670; Becker (2013) ANESTHESIA PROGRESS, 60(1): 25-31).

Of the estimated 300 million plus surgical operations performed globally each year (Weiser, et al. (2016) BULL. WORLD HEALTH ORG., 94(3): 201-209), 90% cause moderate to extreme pain with 12% being in the extreme pain category not adequately controlled by steroids NSAIDS or COX-2 drugs (Gan, et al. (2014) CURR. MED. RES. OPIN., 30(1): 149-60); Gan (2017) J. PAIN RES. 10: 2287-2298). Undertreating the pain experienced by these patients can result in a diminished quality of life and, if improper management continues, chronic pain and inflammation can ensue (Kehlet, et al. (2001) BR. J. ANAESTH. 87(1): 62-72).

Opioid analgesics are often used to manage the pain of patients undertreated by steroid NSAID and COX-2 drugs and, despite the known dangers, they remain a mainstay of many physicians (Zhao, et al. (2019) PAIN RES. MANAGE., 2019: 7490801; Lail, et al. (2014) CAN. J. HOSP. PHARM., 67(5): 337-42; Garimella (2013) CLINICS COLON RECTAL SURG., 26(3): 191-196). It is estimated that, on a worldwide basis, 80% of all surgical patients receive opioid analgesics as the fundamental agent for pain relief (Zhao (2019) supra; Wunsch, et al. (2016) JAMA 315(15): 1654-1657).

Of the 300 million surgeries performed globally each year 70 million are performed in the U.S. and, of these, approximately 7 million are eye surgeries. Patients undergoing eye surgery often receive NSAID analgesics and steroids and approximately 1 million patients are prescribed opioids. Despite these treatments, it is believed that pain from eye surgery remains undertreated (Galor, et al. (2015) EYE, 29: 301-312; Pereira, et al. (2017) PAIN PHYSICIAN, 20: 429-436) increasing the risk that the acute surgical pain progresses to chronic conditions including dry eye syndrome, especially in the cases where opioid treatment was required. Dry eye syndrome is a chronic condition where more powerful, but often ineffective immunosuppressant drugs are required (Trattler, et al. (2017) CLIN. OPHTHALMOL., 11: 1423-1430; Shtein (2011) EXPERT REVIEW OPHTHALMOL., 6(5): 575-582; Schwatrtz (2018) JAMA INTERN. MED., 178(2): 181-182). Today, dry eye syndrome is estimated to occur in approximately 5% of the U.S. population (or about 16.5 million people) and the prevalence is increasing on a yearly basis (Dana, et al. (2019) AM. J. OPHTHALMOL. 202: 47-54).

It is contemplated that, in order to avoid under treatment of surgical pain, while also minimizing reliance on opioid or other analgesic medicines in use today, safer analgesic alternatives of equal of greater efficacy are required which avoid the side effects of todays therapies. Such alternatives could be used either as monotherapies or as part of a multimodal regimen where they would act additively or synergistically with existing therapies (Ong, et al. (2007) CLIN. MED. RES., 5(1):19-34). However, drugs having mechanisms of action different to those currently available are needed for this to occur. As a result, despite the efforts made to date on pain management, especially postoperative pain management, there is still a need for new drugs that address the deficiencies associated with commercially available analgesics.

### SUMMARY OF THE INVENTION

The present invention provides drug compositions, pharmaceutical compositions, formulations, medical kits, and methods of making and using such compositions. The compositions disclosed herein exhibit a different mode of action to NSAIDs, COX-2, opioid and steroid drugs, for example, by agonizing endocannabinoid receptor activity and particularly CB₂ endocannabinoid receptor activity, with dual action for ameliorating pain and inflammation in a subject. The cannabinoid type 2 (CB₂) receptor is a G protein-coupled receptor from the cannabinoid receptor family that, in humans, is encoded by the CNR2 gene.

The compositions described herein display dual analgesic and anti-inflammatory activities and represent a new class of analgesic and/or anti-inflammatory drug. Furthermore, because of their distinct modes of action, the compositions disclosed herein may be combined with drugs that act by different modes of action to achieve a greater total therapeutic effect. The compositions may also enhance the efficacy (synergize the activity) of other drugs that work via different modes of action. As a result, the compositions described herein are capable of acting synergistically with cyclooxygenase inhibitor drugs to ameliorate pain and inflammation. In other words, the compositions described herein have the ability to; (a) reduce the amount of cyclooxygenase inhibitor drug required to achieve a given reduction of pain or inflammation experienced by a subject (COX sparing) or (ii) increase the level pain and inflammation reduction experienced by a subject receiving a cyclooxygenase inhibitor to an extent not achievable by the cyclooxygenase inhibitor alone, or in combination with other drugs that act via the same mode of action (COX enhancement).

In a first aspect, the invention provides a composition, including a composition, for agonizing CB₂ receptor activity in a subject. The composition comprises a combination of:
(a) a first compound of Formula I:

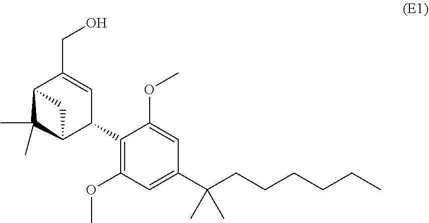 [SMILES: CCCCCCC(C)(C)c1cc(OC)c([C@H]2C=C(CO)[C@H]3C[C@@H]2C3(C)C)c(OC)c1]

or a pharmaceutically acceptable salt thereof; and
(b) a second compound of Formula II:

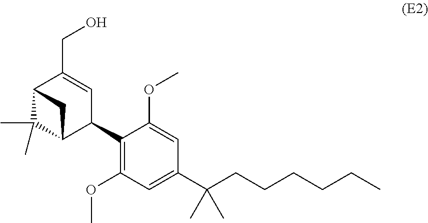 [SMILES: CCCCCCC(C)(C)c1cc(OC)c([C@@H]2C=C(CO)[C@@H]3C[C@H]2C3(C)C)c(OC)c1]

or a pharmaceutically acceptable salt thereof;
where the composition includes the compound of Formula I and the compound of Formula II (which together in combination are referred to as “CB₂ Receptor Agonist Composition” or “CB2RA Composition”) in a weight ratio of from 99.85:0.15 to 93.5:6.5. The pharmaceutical composition also comprises a pharmaceutically acceptable excipient. The weight ratio of the compound of Formula I to the compound of Formula II in the CB2RA Composition of the invention may be from 99.8:0.2 to 98.2:1.8; from 98.8:1.2 to 98.4:1.6 and; from 99.3:0.7 to 98.7:1.3. The weight ratio of the compound of Formula I to the compound of Formula II in the CB2RA Composition of the invention may be about 99:1, or 99:1.

It is contemplated that a pharmaceutical composition comprising the CB2RA Composition may comprise one or more pharmaceutically acceptable excipients to facilitate solubilization and/or delivery of the CB2RA Composition in the pharmaceutical composition. Exemplary excipients include agents that solubilize the CB2RA Composition, including, for example, one or more polymers (for example, amphipathic polymers including non-ionic polymers, poloxomers, diblock and triblock polymers, graft polymers and star shaped polymers), salts, buffers (for example, bicarbonate buffers, citrate buffers, phosphate buffers, tromomethane buffers, lactate buffers and acetate buffers) or a combination thereof. Other excipients that may be used in a pharmaceutical composition described herein include, for example, complex sugars (for example, dextrose, cyclodextrin, fructose, glucose, trehalose and mannitol), oils and waxes (for example, mineral oil, cottonseed oil and soybean oil), stabilizing agents (for example, glycine, histidine or lecithin), viscosity enhancing agents (for example, alginates, carbomers, chitosan, xanthan gum, hyaluronic acid and cellulose derivatives) stabilizing agents (for example, ascorbic acid, cysteine, glutamate and alpha tocoperherol) and preservatives (for example, benzalkonium chloride, benzyl alcohol and sodium benzoate).

It is contemplated that the CB2RA Composition described herein may be provided in variety of forms for storage, delivery or use, including, a dry powder, a lyophilized cake for reconstitution, an injectable, an infusion, a spray, a gel, a cream, an ointment, a bead, a pellet, a particle, a microparticle, a nanoparticle, a micelle, a liposome, a nanomaterial, a compressed nanomaterial, a compressed particle, a compressed powder, a capsule, a tablet, a suppository, a pessary or the like.

Depending upon the intended use, the CB2RA Composition may be formulated into immediate release or modified release forms, for example, where the modified release features include accelerated release, delayed release, extended release, single phase release, dual phase release or multi-phase release of the CB2RA Composition.

Depending upon the circumstances, the CB2RA Composition can be formulated into, for example, micelles, nanoparticles, microparticles, powders or dispersions for delivery. The formulation optionally includes a polymer or series of polymers that facilitate the formation of such micelles, nanoparticles, microparticles, powders or dispersions. In an embodiment, the polymer may be a block co-polymer, such as a polyvinylpyrrolidone-polylactic acid (PVP-PLA) copolymer. An exemplary PVP-PLA copolymer has the structure of Formula III:

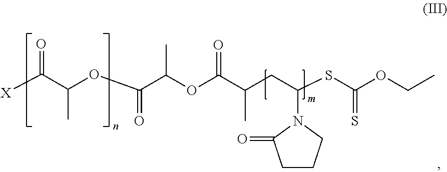

wherein X, n and m, are defined hereinbelow.

It is contemplated that the formulation (e.g., a pharmaceutical composition) comprising the CB2RA Composition may include a buffer or buffering agent, for example, phosphate buffer. Furthermore, it is contemplated that the pharmaceutical composition comprising the CB2RA Composition may also include a salt, to provide a solution suitable for delivery to a subject. Exemplary salts include, for example, a sodium salt, a potassium salt, or a combination thereof.

In certain embodiments, the formulation containing the CB2RA Composition may be in the form of a micellar preparation, which maybe in the form of a liquid or may be in solid form (for example a compressed powder, a lyophilized powder or spray-dried powder). The solid form may be subsequently rehydrated prior to administration to a subject.

In certain embodiments, a pharmaceutical composition may include from about 0.25% (w/w) to about 60% (w/w), from about 0.5% (w/w) to about 40% (w/w), from about 0.75% (w/w) to about 30% (w/w) or from about 1% (w/w) to about 20% (w) of the CB2RA Composition. In addition, the pharmaceutical composition may include from about 5% (w/w) to about 95%, (w/w) or from 30% to about 90%, or from about 60% to about 85% or from about 70% to about 80%. by weight of a polymer. Furthermore, the pharmaceutical composition may include from about 1% to about 20% by weight of a buffering agent. Depending upon the intended modes of delivery and uses, the pharmaceutical composition may also further include a preservative, a complex sugar, a tonicity agent a cryoprotectant, a bulking agent, a solvent or vehicle, a lipid, and a fatty acid. It is contemplated that the actual pharmaceutical composition chosen will depend upon a number of factors including, without limitation, the age and sex of the subject to be treated, the indication and severity of the indication to be treated, the desired release kinetics, and the desired mode of delivery.

In certain embodiments, a solid dosage form, for example, after delivery and storage, may be administered directly to subject as is, or solubilized or rehydrated in a solvent to produce a micellar solution prior to use. The solvent may be, for example, an alcohol, water (for example water for injection), bacteriostatic water, a dextrose solution (for example a 5% dextrose solution), Ringer's solution, Ringer's lactate solution or saline.

The pharmaceutical composition described herein, when in liquid, gel, ointment or other semi-solid form, may have a pH from about 5 to about 9, from about 6 to about 8, or from about 6.5 to about 7.5. The micellar solution of the contemplated composition may include particles having a particle size (Z.av) of between 12-50 nm, 15-45 nm, or 20-40 nm and/or a polydispersity index (PDi) of from about 0.05 to about 0.5. The particles may be charged or uncharged.

The pharmaceutical composition described herein, when in liquid, gel, ointment or other semi-solid form, may have a viscosity range from about 0.2 mPas to about 80,000 mPas, from about 0.5 mPas to about 70,000 mPas, from about 1 mPas to about 60,000 mPas, from about 10 mPas to about 50,000 mPas, from about 50 mPas to about 50,000 mPas, from about 100 mPas to about 50,000 mPas.

Depending upon the circumstances, it is contemplated that the CB2RA Composition may further comprise a third compound, a fourth compound a fifth compound a sixth compound and a seventh compound the amounts of which, with respect to each third, fourth, fifth, sixth and seventh compound, are between 0.015% and 1.5% (wt/wt) of the composition.

Also provided herein is a method of treating inflammation and/or pain in a subject in need thereof, where the method includes administering to the subject a therapeutically effective amount of the CB2RA Composition or a pharmaceutical composition containing the CB2RA Composition. The inflammation or the pain may be chronic or acute. The inflammation or pain may be somatic inflammation or somatic pain which may be postoperative somatic inflammation or post operative somatic pain. The inflammation or pain may be ocular inflammation or ocular pain, which may be postoperative ocular inflammation or postoperative ocular pain. The inflammation or pain may be associated with ocular disease or trauma including corneal trauma (e.g., corneal surgery or injury). The inflammation or pain may be visceral inflammation or visceral pain, which may be postoperative visceral inflammation or postoperative visceral pain. The inflammation or pain may be neuropathic inflammation or neuropathic pain, which may be postoperative neuropathic inflammation or postoperative neuropathic pain.

The method may further comprise administering to the subject a therapeutically effective amount of a nonsteroidal anti-inflammatory drug (NSAID) and/or a steroid. The NSAID may be, for example, aspirin, naproxen, diclofenac, meloxicam, ibuprofen, ketoprofen, tolmetin, indomethacin, sulindac, piroxicam, mefenamic acid, etodolac, flurbiprofen, nepafenac, acetaminophen, bromofenac, ketorolac, celecoxib, etoricoxib, lumiroacoxib, rofecoxib, valdecoxib, parecoxib, acemethacin, dexibuprofen, nimesulide, nabumetone, tiaprofenic acid, lornoxicam, tenoxicam, aceclofenac, proglumethacin, dexketoprofen or oxaprozin. The steroid may be, for example, a corticosteroid, including a glucocorticosteroid. The corticosteroid or glucocorticosteroid may be clobetasol propionate, halobetasol propionate, fluocinonide, diflorasone diacetate, desoximetasone, clocortolone pivalate, mometasone furoate, triamcinolone acetonide, betamethasone valerate, fluticasone propionate, prednicarvate, hydrocortisone probutate, triamcinolone acetonide fluocinolone acetonide, dexamethasone loteprednolloteprednol etabonate, alclometasone dipropionate, desonide or hydrocortisone.

The NSAID and/or steroid may be administered to the subject before, simultaneous with, or after administration of the CB2RA Composition. The administration of the CB2RA Composition may act synergistically with an NSAID and/or steroid or other analgesic or anti-inflammatory drug administered in combination to a subject.

The administration of the CB2RA Composition, when administered in combination with the NSAID and/or steroid or other analgesic or anti-inflammatory drug, may enhance the effect of the NSAID and/or steroid and/or other analgesic and/or anti-inflammatory drug to achieve a greater reduction in pain and inflammation than could be achieved by administering the NSAID and/or steroid and/or other analgesic and/or anti-inflammatory drug alone. The administration of the CB2RA Composition, when administered in combination with the NSAID and/or steroid and or other analgesic or anti-inflammatory drug, may reduce the amount of the NSAID and/or steroid and/or other analgesic and/or anti-inflammatory drug required to achieve a desired reduction in pain and inflammation.

In another aspect, the present invention provides a composition comprising a pharmaceutically effective amount of the CB2RA Composition and a pharmaceutically effective amount of a nonsteroidal anti-inflammatory drug (NSAID) and/or a steroid. The NSAID may be, for example, aspirin, naproxen, diclofenac, meloxicam, ibuprofen, ketoprofen, tolmetin, indomethacin, sulindac, piroxicam, mefenamic acid, etodolac, nepafenac, flurbiprofen, acetaminophen, bromofenac, ketorolac, celecoxib, etoricoxib, lumiroacoxib, rofecoxib, valdecoxib, parecoxib, acemethacin, dexibuprofen, nimesulide, nabumetone, tiaprofenic acid, lornoxicam, tenoxicam, aceclofenac, proglumethacin, dexketoprofen, cylcosporin, lifitegrast, or oxaprozin. The steroid may be, for example, a corticosteroid, including a glucocorticosteroid. The corticosteroid or glucocorticosteroid may be clobetasol propionate, halobetasol propionate, fluocinonide, diflorasone diacetate, desoximetasone, clocortolone pivalate, mometasone furoate, triamcinolone acetonide, betamethasone valerate, fluticasone propionate, prednicarvate, hydrocortisone probutate, triamcinolone acetonide fluocinolone acetonide, dexamethasone loteprednolloteprednol etabonate, alclometasone dipropionate, desonide or hydrocortisone. The CB2RA composition of the invention and the NSAID and/or steroid and or other analgesic drug, and/or other anti-inflammatory drug may be formulated into a single dosage form. The other analgesic drug may be a narcotic drug, a stimulant, an opioid drug, an antidepressant drug or an anticonvulsant drug.

### BRIEF DESCRIPTION OF THE DRAWINGS

FIGS. 1A-F show exemplary rapid ultra-performance convergence (UPC2) chromatograms of various exemplary CB2RA Composition that comprise E1 and E2 at weight ratios of E1 relative to E2 of (a) 98.4:1.6 (FIGS. 1A and 1B), with FIG. 1B being an exploded view of FIG. 1A; (b) 8.4:6.4 (FIGS. 1C and 1D), with FIG. 1D being an exploded view of FIG. 1C; (c) 82.4:17.6 (FIGS. 1E and 1F), with FIG. 1F being an exploded view of FIG. 1E. Each chromatogram was generated using a Waters Acquity UPC2 chromatography system fitted with a Trefoil AMY1 chiral column (2.5 μm, 3.0×150 mm). Each Figure also displays an expanded view of the chromatograms as an inset which expanded view also shows the various other components of each batch separated by their chirality.

FIGS. 2A-B show exemplary expanded view reverse phase HPLC chromatograms of various exemplary CB2RA Compositions with purity (assay) values of 98.9% (FIG. 2A) or 98.7% (FIG. 2B), as determined by reverse phase HPLC. E1 and E2, as enantiomers possessing the same hydrophobicity, are not resolved into separate peaks by reverse phase HPLC. Chromatograms were generated using an Agilent 1100 HPLC system equipped with a quaternary pump, column heating compartment and diode array detector fitted with a Zorbax-Eclipse XDB-C8 5 μm, 15×4.6 mm column. Each expanded view shows both the single peak comprising both E1 (peak 2) and E2 (peak 3) and various other components of these the CB2RA Compositions separated according to their hydrophobicity.

FIG. 3 shows a schematic representation of the process for making an exemplary PVP-PLA based formulation containing an exemplary CB2RA Composition (CB2RA Formulation 1) as used in the rodent incisional pain model.

FIG. 4 shows particle size distribution chromatogram of an exemplary CB2RA Formulation 1 reconstituted with water for injection measured using a Malvern Nano ZS90 Zetasizer. A CB2RA Composition comprising a weight ratio E1 to E2 of 98.4:1.6 was used to generate an exemplary CB2RA Formulation 1 used for this study.

FIG. 5A shows comparison of the withdrawal threshold exhibited by test animals before and after a plantar incision in a model for acute surgical pain.

FIG. 5B shows AUC_{0.25-8 hrs} for withdrawal threshold/time curves after administration of vehicle (saline), increasing concentrations of ketorolac, or a single dose of buprenorphine. PI: plantar incision.

FIG. 5C shows the AUC_{0.25-8 hr}/injected dose, dose/response curve for ketorolac generated from the data in FIG. 5B. An ED₅₀ value of 23.1 mg/kg was calculated for ketorolac as shown in the FIGURE.

FIG. 6A shows rat hindpaw withdrawal threshold (analgesic) response/time plots generated for 2 mg/kg, 4 mg/kg and 11 mg/kg intravenous doses of an exemplary CB2RA Formulation 1 with a weight ratio of E1 to E2 of 99.4:0.6. Withdrawal measurements were made 15 minutes after intravenous administration of the exemplary CB2RA Formulation 1. The withdrawal threshold/time produced by intravenous administration of saline (Control) is also shown. Onset of analgesia, as determined by an increased withdrawal threshold versus Control occurred within 15 minutes of intravenous administration of the CB2RA formulation with analgesia being maintained until the end of the test period for, at least, the 4 mg/kg and 11 mg/kg the groups. The result for group receiving 8 mg/kg of the CB2RA Composition administered in the same manner produced similar results but is omitted from this plot for clarity.

FIG. 6B shows AUC_{0.25-8 hr} for Control and 2 mg/kg, 4 mg/kg, 8 mg/kg and 11 mg/kg intravenous doses of an exemplary CB2RA Formulation 1 of FIG. 5A calculated by analysis of the withdrawal threshold/time plots. AUC_{0.25-8 hr} values from 2 mg/kg to 8 mg/kg the CB2RA Composition increased in proportion to administered dose with 8 mg/kg and 11 mg/kg doses exhibiting a similar or ‘plateau’ response. Based on these data, the 4 mg/kg the CB2RA composition dose, being central to the dose/response curve, was selected to determine the effect of E1 to E2 weight ratio on analgesic response in subsequent experiments.

FIG. 7 shows the AUC_{0.25-8 hr}/injected dose, dose/response curve for the exemplary CB2RA Composition of FIG. 6B. An ED₅₀ value of 3.0 mg/kg was calculated for this CB2RA Composition as shown in the FIGURE.

FIG. 8 shows AUC_{0.25-8 hr} for 2 mg/kg and 4 mg/kg doses of a CB2RA Formulation 1 with a weight ratio of E1 to E2 of 98.8:1.2. A dose/response effect was observed between the 2 mg/kg and 4 mg/kg doses of the CB2RA Composition with the AUC_{0.25-8 hr} for the 4 mg/kg dose being greater than that generated in this model by the CB2RA Composition comprising an E1 to E2 ratio of 99.4:0.6. The AUC_{0.25-8 hr} for the 2 mg/kg dose was also greater than that generated with the CB2RA composition comprising an E1 to E2 ratio of 99.4:0.6.

FIG. 9 shows AUC_{0.25-8 hr} for 2 mg/kg and 4 mg/kg doses of a CB2RA Formulation 1 with a weight ratio of E1 to E2 of 98.4:1.6. A dose/response relationship was observed between the 2 mg/kg and 4 mg/kg doses. While still analgesic versus Control, the AUC_{0.25-8 hr} for the 4 mg/kg dose of this CB2RA Composition was less than that generated in this model for the CB2RA composition comprising E1 to E2 ratios of either 99.4:0.6 or 98.8:1.2. The AUC_{0.25-8 hr} for the 2 mg/kg dose of this CB2RA composition was also lower than that generated with the CB2RA Composition comprising E1 to E2 ratios of either 99.4:0.6 or 98.8:1.2.

FIG. 10 shows AUC_{0.25-8 hr} for 2 mg/kg and 4 mg/kg doses of a CB2RA Formulation 1 with a weight ratio of E1 to E2 of 93.6:6.4. Here, while analgesia was observed for both doses versus Control, no dose/response relationship was observed between the 2 mg/kg and 4 mg/kg doses. AUC_{0.25-8 hr} for the 4 mg/kg dose of this CB2RA Composition was also less than that generated in this model for the CB2RA Composition comprising weight ratios of E1 to E2 of either 99.4:0.6, 98.8:1.2 or 98.4:1.6. The AUC_{0.25-8 hr} for the 2 mg/kg dose of this the CB2RA Composition was lower than that generated with the CB2RA composition comprising weight ratios of E1 to E2 ratio of 99.4:0.6 or 98.8:1.2, but was similar to those generated by the CB2RA composition comprising an E1 to E2 ratio of 98.8:1.6.

FIG. 11 shows AUC_{0.25-8 hr} for 2 mg/kg and 4 mg/kg doses of a CB2RA Formulation 1 comprising the CB2RA Composition with weight ratio of E1 to E2 of 82.4:17.6. Analgesia was again observed for both doses versus Control but again no dose/response relationship was observed between the 2 mg/kg and 4 mg/kg doses. Results at the applied dose were similar to those generated with the CB2RA composition comprising an E1 to E2 weight ratio of 93.6:6.4.

FIG. 12A compares the AUC_{0.25-8 hr} for the 4 mg/kg intravenous doses of the various CB2RA Compositions shown in FIGS. 7, 8, 9, 10 and 11 demonstrating the change in AUC_{0.25-8 hr} (or analgesic effect) as a function of the E1 to E2 weight ratio in each CB2RA Composition. While all doses of each CB2RA Composition administered to animals, irrespective of E1 to E2 ratio, displayed an analgesic effect versus Control, the analgesic effect was greatest for the CB2RA Composition comprising an E1 to E2 ratio of 98.8:1.2.

FIG. 12B shows curve fitting analysis of the data in FIG. 12A highlighting that all compositions of the CB2RA Composition tested generated analgesia greater than Control (Vehicle) and the range of E2 ratios (between 0.19 and 1.78) where the enhancing analgesic effect of E2 was observed. Curve fitting predicted that the greatest degree of enhanced analgesic effect occurs at a weight ratio E1 to E2 of about 99:1 where Area under the curve was double that of other E1 to E2 compositions.

FIG. 13 shows the plasma concentrations over time of an exemplary CB2RA Composition (comprising E1 to E2 in the weight ratio of 99.4:0.6) following intravenous administration of an exemplary CB2RA formulation to male Sprague Dawley rats. The CB2RA Composition concentrations in rat plasma, for the first 12 hours after dosing, were determined by GC-MS/MS using an Agilent HP-5 ms UltraInert (30 m, 0.25 mm, 0.25 μm) analytical column employing ethyl acetate as the mobile phase.

FIG. 14 shows AUC_{0.25-8 hr} for Control and 15 mg/kg, 22.5 mg/kg, 30 mg/kg and 40 mg/kg intravenous doses of a micellar PVP-PLA composition of the COX inhibitor, celecoxib (COX Formulation 1) administered to rats 1 hour post hind paw incisional surgery as performed for analysis of the CB2RA formulations described in Example. AUC_{0.25-8 hr} values from 15 mg/kg to 30 mg/kg increased in proportion to administered dose with 30 mg/kg and 40 mg/kg celecoxib doses exhibiting a similar ‘plateau’ response.

FIG. 15 shows the AUC_{0.25-8 hr}/injected dose, dose/response curve for a celcoxib formulation (COX Formulation 1) as generated from data presented in FIG. 14. An ED₅₀ value of 22.29 mg/kg was calculated for celecoxib delivered by PVP-PLA micelles.

FIG. 16 shows AUC_{0.25-8 hr} values for Control and increasing doses of a fixed ratio mixture of CB2RA Formulation 1 and COX Formulation 1 (collectively referred to as CB2RA Formulation 2) administered to rats post surgery as described in Example 5. The CB2RA Formulation 1 used for this study contained a CB2RA Composition having a weight ratio of E1 to E2 of 99.4:0.6. The two formulations, namely CB2RA Formulation 1 and COX Formulation 1, were mixed in liquid form to produce CB2RA Formulation 2 at a weight ratio of approximately 1:13 the CB2RA Composition to celecoxib based on their relative ED₂₅ values calculated from their respective dose/response curves shown in FIGS. 7 and 15. The doses of CB2RA Formulation 2 administered ranged from 0.32 mg/kg to 1.125 mg/kg with respect to the CB2RA Composition and 4.22 mg/kg to 15 mg/kg with respect to celecoxib. All doses of CB2RA Formulation 2 generated an analgesic response superior to control and a dose response relationship was seen from the lowest to the highest dose administered.

FIG. 17 shows the dose/response curve generated from FIG. 16 with respect to the administered dose of the CB2RA Composition as generated by CB2RA Formulation 2 (▪) and compared with the dose/response curve of the CB2RA Composition as generated by CB2RA formulation 1 from FIG. 7 (●). When combined with celecoxib, the dose/response curve for the CB2RA Composition is shifted to the left predicting an ED₅₀ for the CB2RA Composition of 0.42 mg/kg when administered as the fixed dose combination, compared to 5.6 mg/kg for the CB2RA Formulation 1.

FIG. 18 shows dose/response curve generated from FIG. 16 with respect to the administered dose of celecoxib as generated by CB2RA Formulation 2 (▪) and compared with the dose/response curve of celecoxib as generated by the COX Formulation 1 from FIG. 15 (●). When combined with the CB2RA Composition the dose/response curve for celecoxib is shifted to the left predicting an ED₅₀ for celecoxib of 5.6 mg/kg when administered as the fixed dose combination, compared to 22.29 mg/kg for the COX Formulation 1.

FIG. 19 shows the ED₅₀ value generated for the CB2RA Composition by CB2RA Formulation 1, the ED₅₀ value generated for celecoxib by COX Formulation 1 and the ED₅₀ value generated for the mixture of the CB2RA Formulation 1 and the COX Formulation 1 in the form of CB2RA Formulation 2. The ED₅₀ value for the CB2RA Composition as CB2RA Formulation 1 (2.9 mg/kg) is marked on the x axis and joined by a straight line (isobole) to the ED₅₀ value for celecoxib in COX Formulation 1 (22.29 mg/kg) plotted on the y axis. The connecting isobole represents the ED₅₀ values that would be generated by the fixed dose combination ratios if the analgesic effects of the CB2RA Composition and celecoxib were simply additive. If the ED₅₀ value the fixed dose combination were to fall above the isobole then the combined effect of the CB2RA Composition and celecoxib would be antagonistic in nature. As the ED₅₀ value of the fixed dose combination fell below the isobole then the combined effect of the CB2RA Composition and celecoxib is synergistic. In other words, the combined effect is greater than the sum of the individual drug effects for any given dose.

FIG. 20 is a bar chart showing that the topical administration of a CB2RA composition formulation comprising PVP-PLA reduces corneal pain in BALB/c mice after corneal cauterization and capsaicin challenge, when performed according to the protocol set forth in Thapa D. et al. (2018) CANNABIS CANNABINOID RES. 3:1, 11-20. Data (as pain scores) were compared to those generated by a vehicle control or a 1.5% solution of HU-308 formulated in soybean oil. Pain score measurements to determine the analgesic effects of the various test agents were recorded electronically for 30 seconds following capsaicin challenge and then reviewed blind. Data were then unblinded and plotted as shown in the Figure.

FIG. 21 (A) is a bar chart showing the number of neutrophils per section in corneas from mice treated with a 0.5% PVP-PLA formulation of a CB2RA composition formulation at 12 hours post-injury compared to vehicle-treated eyes visualized and generated according to the method of Thapa (2018) supra. The results were compared against data generated after application of a 1.5% solution of HU-308 formulated in soybean oil. Representative images of transverse sections of the central cornea treated with vehicle are shown in FIG. 21(B) and with a 0.5% CB2RA formulation are shown in FIG. 21(C); the scale bar=50 μm.

FIG. 22 is a bar chart showing that topical administration of a CB2RA Formulation reduces corneal pain in BALB/c mice after corneal cauterization and capsaicin challenge performed according to the method of Thapa D. et al. (2018)². Method as per FIG. 20. Values represent mean+SEM. For statistical analysis, one-way ANOVA with Dunnett's post hoc test (compared to vehicle) was used. **p<0.01, *p<0.05.

FIG. 23 is a graph fitting of the data from Table 30 using a cubic spline function for ED₅₀ determination of TA-A001 in murine corneal hyperalgesia model.

FIG. 24 is a bar chart showing that topical administration of a COX formulation does not reduce corneal pain in BALB/c mice after corneal cauterization and capsaicin challenge performed according to the method of Thapa D. et al. (2018). Pain score measurements to determine the analgesic effects of the test agents were recorded electronically for 30 seconds following capsaicin challenge and then reviewed blind. Data were then unblinded and plotted as shown in the Figure.

FIG. 25 is a bar chart showing the effects of intraperitoneal application of CB2RA formulation on the amount of IBA-1 positive cells in the subretinal space by quantification of IBA-1 positive cells in the subretinal space of mice exposed to blue light and treated with saline, vehicle, or different concentrations of the CB2RA formulation (n=5-8).

FIG. 26A shows the degree of neovascularization generated by PBS, vehicle, anti-VEGF, the different concentrations of the CB2RA composition, celecoxib or the mixture of the CB2RA composition and celecoxib. Change in vascular area T24-T0 normalized to the PBS (*** P<0.001 vs PBS, n=4-7) is depicted.

FIG. 26B shows the same results as shown in FIG. 26A normalized to the PBS response (test article response/PBS response). Variation of vascular area presented as a fold change of PBS condition. (** P<0.01, *** P<0.001 vs vehicle) is shown.

FIG. 27A is a line graph showing fluorescence intensity above the baseline plotted as a function of time for vehicle, 0.25 and 0.5% of CB2RA. Data were fitted using monoexponential decay equations.

FIG. 27B is a bar chart showing gealing rate constants calculated from fitting the data for vehicle, 0.25 and 0.5% CB2RA.

FIG. 28 is a bar chart showing leukocyte numbers in the cornea of rats exposed to saline, PVP-PLA polymer or increasing concentrations of the CB2RA Composition (as the CB2RA Formulation). Leukocyte numbers were measured 8 hrs after cauterization with silver nitrate.

FIG. 29 is a bar chart showing concentrations of CB2RA Composition in ng/100 mg in front of eye homogenates collected 6 hrs after final dosing with CB2RA Formulation. Results are presented means±SEM. Statistical differences were evaluated using two-way ANOVA with Tukey's post-hoc test. * p<0.05, ** p<0.01, *** p<0.001.

FIG. 30 is a bar chart showing concentrations of CB2RA Composition in ng/100 mg in back of eye homogenates collected 6 hrs after final dosing with CB2RA Formulation. Results are presented means±SEM. Statistical differences were evaluated using two-way ANOVA with Tukey's post-hoc test. * p<0.05, ** p<0.01, *** p<0.001.

### DETAILED DESCRIPTION

The invention is based, in part, upon the discovery of a new composition of matter, the CB2RA Composition with a unique mode of action, distinct from those of currently available anti-inflammatory and/or analgesic medicines, namely agonism of the cannabinoid CB₂ receptor. The composition described herein represents a new composition for treating pain and/or inflammation in a subject and represents a first in class endocannabinoid receptor-mediated dual analgesic and anti-inflammatory agent. The invention thus provides therapeutic compositions, therapeutic formulations, therapeutic dosage forms, medical kits, and methods of making and using such compositions, therapeutic dosage forms and medical kits.

The practice of the present invention employs, unless otherwise indicated, conventional techniques of organic chemistry, pharmacology, and biochemistry. Such techniques are explained in the literature, such as in “Comprehensive Organic Synthesis” (B. M. Trost & I. Fleming, eds., 1991-1992), each of which is herein incorporated by reference in its entirety. Various aspects of the invention are set forth below in sections; however, aspects of the invention described in one particular section are not to be limited to any particular section.

### I. Definitions

To facilitate an understanding of the present invention, a number of terms and phrases are defined below.

The terms “a” and “an” as used herein mean “one or more” and include the plural unless the context is inappropriate.

The compounds and compositions of the disclosure may contain one or more chiral centers and/or double bonds and, therefore, exist as stereoisomers, such as geometric isomers, enantiomers or diastereomers. The term “stereoisomers” when used herein consists of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

Individual stereoisomers of compounds of the present invention may be prepared synthetically from commercially available starting materials that may contain, or be caused by processing to contain, asymmetric or stereogenic centers. Conversely, such starting materials may require novel methods of synthesis in order to precisely control stereoisomeric ratios in the compounds. The individual stereoisomers of compounds of the present invention may also be obtained by preparation of stereoisomeric mixtures followed by resolution methods well known to those of ordinary skill in the art. These methods of resolution are exemplified by direct separation of a mixture of optical enantiomers on chiral chromatographic columns. Stereoisomeric mixtures can also be resolved into their component stereoisomers by well-known methods, such as chiral-phase gas chromatography or chiral-phase high performance liquid chromatography. Further, enantiomers can be separated using supercritical fluid chromatographic (SFC) techniques described in the literature. Still further, stereoisomers can be obtained from stereomerically-pure intermediates, reagents, and catalysts by well-known asymmetric synthetic methods.

Geometric isomers can also exist in the compounds of the present invention. The symbol [symbol] denotes a bond that may be a single, double or triple bond as described herein. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The invention also embraces isotopically labeled compounds of the invention which are identical to those recited herein, except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the invention can generally be prepared by following procedures analogous to those disclosed in, e.g., the Examples herein by substituting an isotopically labeled reagent for a non-isotopically labeled reagent.

As used herein, the terms “subject” and “patient” refer to organisms to be treated by the methods of the present invention. Such organisms are preferably mammals (e.g., murines, simians, equines, bovines, porcines, canines, felines, and the like), and more preferably humans.

As used herein, the term “effective amount” refers to the amount of a compound or composition (e.g., a compound or composition of the present invention) sufficient to effect beneficial or desired results. An effective amount can be administered by one compound alone or two or more compounds working together, in one or more administrations, applications or dosages and is not intended to be limited to a particular formulation or administration route. As used herein, the terms “treat,” “treating,” and “treatment” include any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Martin, Remington's Pharmaceutical Sciences, 15th Ed., Mack Publ. Co., Easton, PA [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

The phrase “therapeutically-effective amount” as used herein means that amount of a compound, material, or composition (for example, a composition comprising a compound or compounds of the present invention) which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. In the case of differences in ratios of stereoisomers in the present invention, one to the other, then the term ‘about’ before the specific quantitative value also includes the specific quantitative value itself unless specifically stated otherwise.

It should be understood that the order of steps or order for performing certain actions is immaterial so long as the present invention remain operable. Moreover, two or more steps or actions may be conducted simultaneously.

At various places in the present specification, substituents are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl. By way of other examples, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20. Additional examples include that the phrase “optionally substituted with 1-5 substituents” is specifically intended to individually disclose a chemical group that can include 0, 1, 2, 3, 4, 5, 0-5, 0-4, 0-3, 0-2, 0-1, 1-5, 1-4, 1-3, 1-2, 2-5, 2-4, 2-3, 3-5, 3-4, and 4-5 substituents.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

Abbreviations as used herein include mass spectrometry (MS), gas chromatography (GC), retention times (RT), relative retention times (RRT), supercritical fluid chromatographic (SFC), and high-performance liquid chromatography (HPLC).

### II. Compositions and Pharmaceutical Compositions

The present invention is, in part, directed to a new chemical composition that, as a CB₂R agonist, affords a new mechanism of action for simultaneously treating pain and/or inflammation in a subject.

In one aspect, provided herein is a composition, for example, a pharmaceutical composition, capable of agonizing CB₂ receptor activity in a subject. The composition comprises a combination of:
(a) a first compound of Formula I:

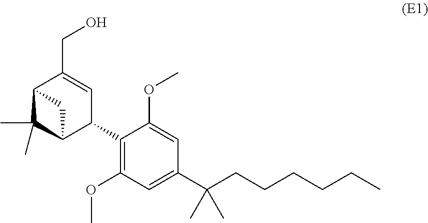 [SMILES: CCCCCCC(C)(C)c1cc(OC)c([C@H]2C=C(CO)[C@H]3C[C@@H]2C3(C)C)c(OC)c1]

or a pharmaceutically acceptable salt thereof;
and
(b) a second compound of Formula II:

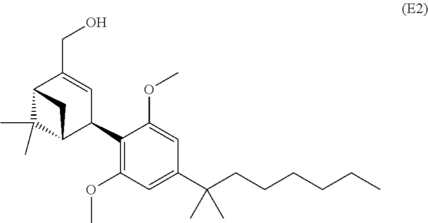 [SMILES: CCCCCCC(C)(C)c1cc(OC)c([C@@H]2C=C(CO)[C@@H]3C[C@H]2C3(C)C)c(OC)c1]

or a pharmaceutically acceptable salt thereof;
wherein the composition comprises the compound of Formula I and the compound of Formula II in a weight ratio of from 99.85:0.15 to 93.5:6.5. The pharmaceutical composition also comprises a pharmaceutically acceptable excipient.

A composition comprising both a first compound of Formula I and a second compound of Formula II is herein referred to as the CB2 receptor agonist Composition or a CB2RA Composition. The compound of Formula I is also herein referred to as Enantiomer 1 (“E1”) and is based on the CB₂ receptor agonist known as Hu-308 (Soethoudt (2017) NATURE COMMUN., 8: 13958). The compound of Formula II is also herein referred to as Enantiomer 2 (“E2”) and is based on the CB₂ receptor agonist known as Hu-433 (Morales, et al. (2017) FRONT. PHARMACOL., 8: 422). The E2 enantiomer reportedly has a potency 1,000 to 10,000 times greater than E1 while binding the same receptor but yet possesses a lower affinity for CB₂ receptor than E1 (Smoum et al. (2015) PROC. NATL. ACAD. SCI. USA, 112.28: 8774-8779).

In describing this new chemical entity, the activity profiles of enantiomeric compositions comprising different ratios of the first and second compounds known as E1 and E2 are provided. These ratios may be defined in terms of weight ratios, one enantiomer to the other (wt/wt), or in terms of enantiomeric excess, where the enantiomeric excess is calculated using the following formula (E1-E2)/(E1+E2).

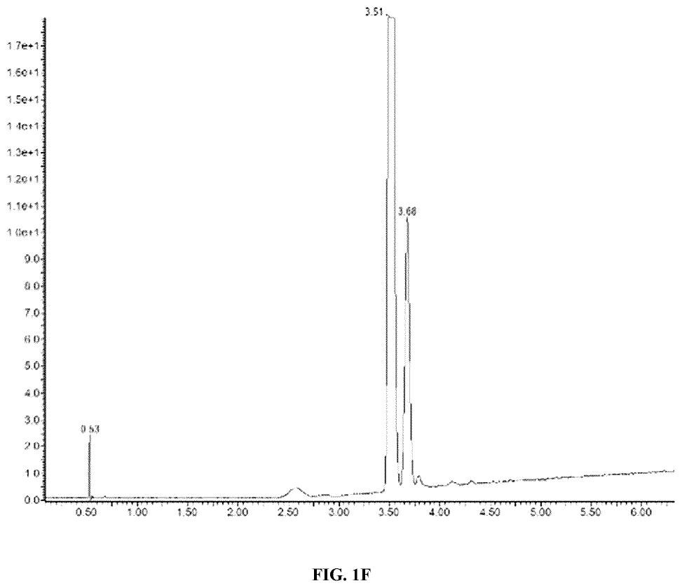

The present invention is based, in part, on an unexpected discovery that a CB₂ receptor agonist composition comprising a combination of the compound of Formula I (E1) and the compound of Formula II (E2) displays different degrees of analgesic and/or anti-inflammatory effect in a model of acute pain and inflammation depending on the weight ratio of the enantiomers E1 and E2 (see, FIGS. 6B and FIGS. 8-11).

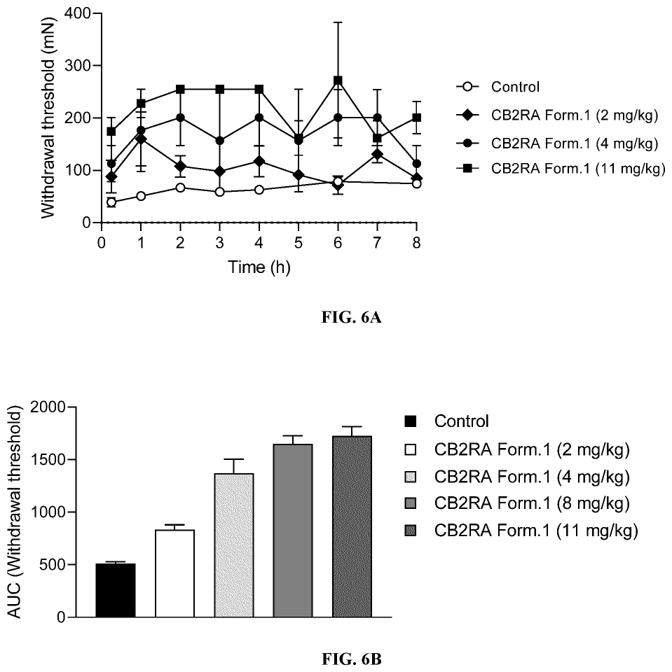

Furthermore, it has been discovered, unexpectedly, that despite E2 reportedly displaying potency 1,000 to 10,000 times greater than that of E1 when administered alone to an animal (Smoum et al. (2015) supra), when in the presence of E1, and when E2 amounts are increased relative to amounts of E1 in the composition, the change in analgesic response does not reflect the 1,000-10,000 fold potency increase reported. Rather, it has been discovered that optimal activity can be achieved using specific ratios of E1 and E2, where E1 is in excess of E2. In other words, the present invention is based, in part, on the unexpected discovery that despite the reportedly greater potency of E2 over E1, the potency of the CB2RA Composition of the invention does not continue to increase as the amount of E2 in the composition increases but instead that potency increases are limited to a defined range of E2 concentrations in the enantiomeric mixture (FIG. 12A). While the CB2RA Compositions of the invention with weight ratios of E1 to E2 described herein provided an analgesic and/or anti-inflammatory response greater than that generated by Control administration, those with a defined range of weight ratios provided an enhanced analgesic response relative to other ratios.

Thus, and for example, it has been discovered through curve fitting (see, FIG. 12B) that, when the amount of E2 in the CB2RA Composition is between about 0.15% to about 2% or between about 0.2% to about 1.8% then the analgesic potency of the CB2RA Composition (e.g., the analgesic effect per milligram of CB2RA Composition) is increased. When the weight ratio of E1 to E2 is about 99:1, the potency of the CB2RA Composition is over twice that of CB2RA Compositions with an amount of E2 outside this range. Such enhanced potency is greatly beneficial to a subject requiring analgesia because a lower dose of the CB2RA Composition can be administered to achieve the required level of analgesia. As is well known in the art, lower doses of drugs such as analgesics drugs, are associated with fewer adverse side effects. Patient life quality may therefore be increase if a higher potency CB2RA Composition is administered.

Conversely, because of their higher potency CB2RA Compositions with an amount of E2 between about 0.15% to about 2% or between about 0.2% to about 1.8% can achieve more analgesia per dose than CB2RA Compositions with amounts of E2 outside this range. Thus, for a given level of adverse side effects, greater analgesic relief can be achieved when administering a CB2RA Composition with an amount of E2 between about 0.15% to about 2% or between about 0.2% to about 1.8% than could be achieved with a CB2RA Composition with an amount of E2 outside this range. This can be beneficial to a subject in need of analgesia as greater relief can be obtained.

The increased potency achieved by CB2RA Compositions with amounts of E2 between about 0.15% to about 2% or between about 0.2% to about 1.8% may therefore have a greater ceiling dose than CB2RA Compositions with amounts of E2 outside this range.

Additionally, it has been discovered that the CB2RA Compositions described herein may act synergistically when mixed with non-CB2RA Composition e.g., a cyclooxygenase inhibitor analgesic, to create a pharmaceutical composition of greater potency than either of the cyclooxygenase inhibitor, or the CB2RA Composition, alone. It is contemplated that this synergistic effect will be greatly beneficial to a subject in need of an analgesic and or anti-inflammatory effect especially in the multi-modal setting. It is contemplated that those in need of analgesic and or anti-inflammatory relief can derive greatest benefit from the co-administration of pharmaceutical agents possessing different but complementary modes of action whether administered separately or as an admixture. The synergy of the CB2RA Compositions described herein with inhibitors of cyclooxygenase enzymes (e.g., COX inhibitors) thus allows greatly reduced doses of either the CB2RA Composition, the cyclooxygenase inhibitor, or both agents to be administered in order to provide a satisfactory analgesic and or an anti-inflammatory effect than would be required if the CB2RA Composition or the cyclooxygenase inhibitor was administered alone, or if a purely additive analgesic effect was achieved by their mixing. Fewer adverse side effects may therefore be generated by the multi-modal synergistic combination resulting in an improved treatment outcome for the subject and a better subject experience. Conversely, the greater potency achieved by combining, or otherwise co-administering the CB2RA Composition, and the cyclooxygenase inhibitor, allows greater amounts of analgesic and or anti-inflammatory effect for any given dose of CB2RA Composition, or cyclooxygenase inhibitor if given alone. The discovery that the CB2RA Composition of the present invention can act synergistically with cyclooxygenase enzyme inhibitors therefore allows greater levels of analgesia and anti-inflammatory effect to be achieved safely.

A new enantiomeric CB₂ receptor mediated analgesic and/or anti-inflammatory agent, namely the CB2RA Composition described herein has been discovered with a mode of action different to the commercially available opioid receptor agonists, cyclooxygenase inhibitors and steroids commonly used to treat pain and inflammation respectively, which can act synergistically with commonly used NSAID analgesics. Furthermore, it has been discovered unexpectedly that the ratio of enantiomers in the CB2RA Composition has a profound effect on the potency of the composition such that a range of ratios may be defined wherein the combined effect of the enantiomers results in an increased potency that may reduce by half the amount of the CB2RA Composition required to treat a subject. In addition, it has been discovered that, when formulated using a micellar technology (for example, using a PVP-PLA-based polymer) and delivered intravenously, the CB2RA Composition elicits an immediate and prolonged analgesic and/or anti-inflammatory effect in a model of acute incisional pain with a potency, in terms of ED₅₀ approximately ten times that of commercially available NSAIDS and COX-2 inhibitors (see, for example, Table 3).

In view of the foregoing, the contemplated compositions may have a weight ratio of the compound of Formula I (E1) to the compound of Formula II (E2) of from 99.85:0.15 to 93.5:6.5, from 99.8:0.2 to 98.2:1.8, from 98.8:1.2 to 98.4:1.6 and 99.3:0.70 to 98.7:1.3. The weight ratio of the compound of Formula I to the compound of Formula II in the CB2RA Composition may be about 99:1, or 99:1.

For convenience, the values set forth in weight ratio ranges of E1:E2 and the equivalent values set forth in terms of enantiomeric excess ranges are summarized in Table 1.

TABLE 1

| Weight Ratio Range (E1:E2) | Enantiomeric Excess Range¹ |
|---|---|
| 99.85:0.15 to 93.5:6.5 | 99.7-87.0 |
| 99.8:0.2 to 98.22:1.8 | 99.6-96.4 |
| 99.3:0.7 to 98.7:1.3 | 98.6-97.4 |
| 98.8:1.2 to 98.4:1.6 | 97.6-96.8 |
| 99:1 | 98 |

¹EE % = (|E1 − E2|)/(E1 + E2) *100

Furthermore, in certain embodiments, the weight ratio of the compound of Formula I to the compound of Formula II is or about 99:1, is or about 98:2, is or about 97:3, is or about 96:4, is or about 95:5, or is or about 94:6. These values correspond to EE values of about 98, about 96, about 94, about 92, about 90 and about 88, respectively.

Similarly, the CB2RA Compositions described herein may have an EE of from about 87% to about 99.7%, from about 96.4% to about 99.6%, from about 97.4% to 98.6%, from about 88% to 98%, from about 90% to about 98%, from about 92% to about 98%, from about 94% to about 98%, from about 96% to about 98%, favoring the compound of Formula (I) relative to the compound of Formula (II). The composition described herein may have an EE of about 87%, about 88%, about 90%, about 92%, about 94%, about 96%, or about 98%, favoring the compound of Formula (I) relative to the compound of Formula (II).

The composition may further include from 0.015% to 1.5% or 0.01% to 1% of a third, fourth, fifth or sixth compound with retention times (RT) and relative retention times (RRT), as generated according to the reverse phase HPLC analytical method of Example 2 as specified in Table 2. In Table 2, Content (%) denotes the measured amount of compound in terms of percentage composition relative to that of the entire composition and (+) denotes an identified compound with a percentage composition relative to that of the entire composition below the level of quantification of the specified analytical method.

TABLE 2
Compound Number

| | | | | CB2RA | | |
|---|---|---|---|---|---|---|
| | | | | Com- | | |
| 1 | 2 | 3 | 4 | position | 6 | 7 |

Retention Time (RT min.)

| | | | | | | |
|---|---|---|---|---|---|---|
| 17.4 | 18.1 | 18.4 | 19.2 | 19.9 | 20.2 | 23.2 |

Relative Retention Time (RRT. min)

| | | | | | | |
|---|---|---|---|---|---|---|
| 0.87 | 0.91 | 0.92 | 0.96 | 1.00 | 1.02 | 1.16 |

Content (%)

| | | | | | | | |
|---|---|---|---|---|---|---|---|
| Lot 1 | 0.32 | + | 0.65 | + | 98.18 | 0.13 | 0.08 |
| Lot 2 | 0.54 | 0.11 | 0.29 | 0.13 | 98.01 | 0.09 | + |

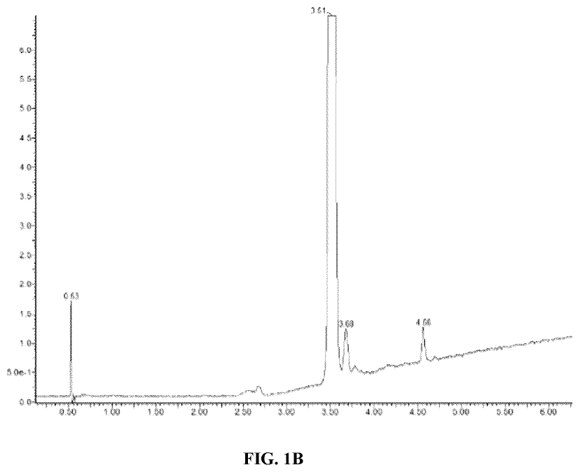

Table 2 shows the existence of certain compounds in addition to the CB2RA Composition in two exemplary lots of material as produced in the Example 9, their peak number, their percentage compositions relative to the CB2RA Composition, and their retention times as identified by the reverse phase HPLC method as described in Example 2 of FIG. 2A. The CB2RA Composition (i.e., containing enantiomers E1 and E2) has a retention time of 19.9 minutes using the reverse phase HPLC method cited for FIG. 2, and (+) denotes the presence of compound other than the CB2RA composition where the percentage composition was below levels of quantification of the reverse phase HPLC method. A first compound with a peak at 17.4 minutes is present in the amount from 0.1% to 0.6%, a second compound with a peak at 18.1 minutes is present in the amount of 0.01% to 0.2%, a third compound with a peak at 18.4 minutes is present in the amount of 0.2% to 0.7%, a fourth compound with a peak at 19.2 minutes is present in the amount of 0.01 to 0.15%, a sixth compound with a peak at 20.2 minutes is present in the amount of 0.05% to 0.15%, and a seventh compound with a peak at 23.2 minutes is present in the amount of 0.01% to 0.1%.

In one embodiment, the composition comprises up to 15% wt/wt of an impurity which has a structure of Formula B:

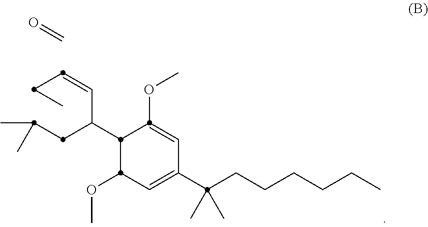

In one embodiment, the composition comprises up 14% wt/wt of the impurity. In one embodiment, the composition comprises up 13% wt/wt of the impurity. In one embodiment, the composition comprises up 12% wt/wt of the impurity. In one embodiment, the composition comprises up 11% wt/wt of the impurity. In one embodiment, the composition comprises up 10% wt/wt of the impurity. In one embodiment, the composition comprises up 9% wt/wt of the impurity. In one embodiment, the composition comprises up 8% wt/wt of the impurity. In one embodiment, the composition comprises up 7% wt/wt of the impurity. In one embodiment, the composition comprises up 6% wt/wt of the impurity. In one embodiment, the composition comprises up 5% wt/wt of the impurity. In one embodiment, the composition comprises up 4% wt/wt of the impurity. In one embodiment, the composition comprises up 3% wt/wt of the impurity. In one embodiment, the composition comprises up 2% wt/wt of the impurity. In one embodiment, the composition comprises up 1% wt/wt of the impurity. In one embodiment, the composition comprises up 0.5% wt/wt of the impurity. In one embodiment, the composition comprises up 0.1% wt/wt of the impurity. In one embodiment, the composition comprises up 0.05% wt/wt of the impurity. In one embodiment, the composition comprises up 0.01% wt/wt of the impurity. In one embodiment, the composition comprises up 0.005% wt/wt of the impurity. In one embodiment, the composition comprises up 0.001% wt/wt of the impurity.

In some embodiments, the amount of the impurity measured above is following incubation at 40° C. In some embodiments, the amount of the impurity measured above is following incubation at 40° C. for 1 month. In some embodiments, the amount of the impurity measured above is following incubation at 40° C. for 2 months. In some embodiments, the amount of the impurity measured above is following incubation at 40° C. for 3 months. In some embodiments, the amount of the impurity measured above is following incubation at 40° C. for 6 months.

In some embodiments, the amount of the impurity measured above is following incubation at 65° C. In some embodiments, the amount of the impurity measured above is following incubation at 65° C. for 24 hours. In some embodiments, the amount of the impurity measured above is following incubation at 65° C. for 48 hours.

In some embodiments, the amount of the impurity measured above is following incubation at 80° C. In some embodiments, the amount of the impurity measured above is following incubation at 80° C. for 24 hours. In some embodiments, the amount of the impurity measured above is following incubation at 80° C. for 48 hours.

In one embodiment, the composition further comprises an antioxidant for reducing formation of the impurity.

In one embodiment, the antioxidant is selected from the group consisting of ascorbic acid, butylated hydroxytoluene, sesamol, guaiac resin, methionine, citric acid, tartaric acid, phosphoric acid, thiol derivatives, potassium metabisulphite, ascorbyl palmitate, calcium stearate, propyl gallate, sodium thiosulphate, glutathione, dihydroxybenzoic acid, benzoic acid, urate and uric acid, sorbic acid, sodium benzoate, EDTA, sodium bisulphite, vitamin E, cysteine hydrochloride, sorbitol, butylated hydroxyanisol, and mixtures thereof.

In one embodiment, the antioxidant is selected from the group consisting of EDTA, sodium bisulphite, vitamin E, cysteine hydrochloride, sorbitol, butylated hydroxyanisol, and mixtures thereof.

In one embodiment, the antioxidant comprises butylated hydroxyanisol.

In one embodiment, the composition is essentially free of oxygen.

In one embodiment, the composition is free of oxygen.

In one embodiment, the composition is stored under an inert gas.

### III. Synthesis of Active Ingredient(s)

E1 and E2 can be synthesized according to the Schemes shown below and also the methods as described in Example 9.

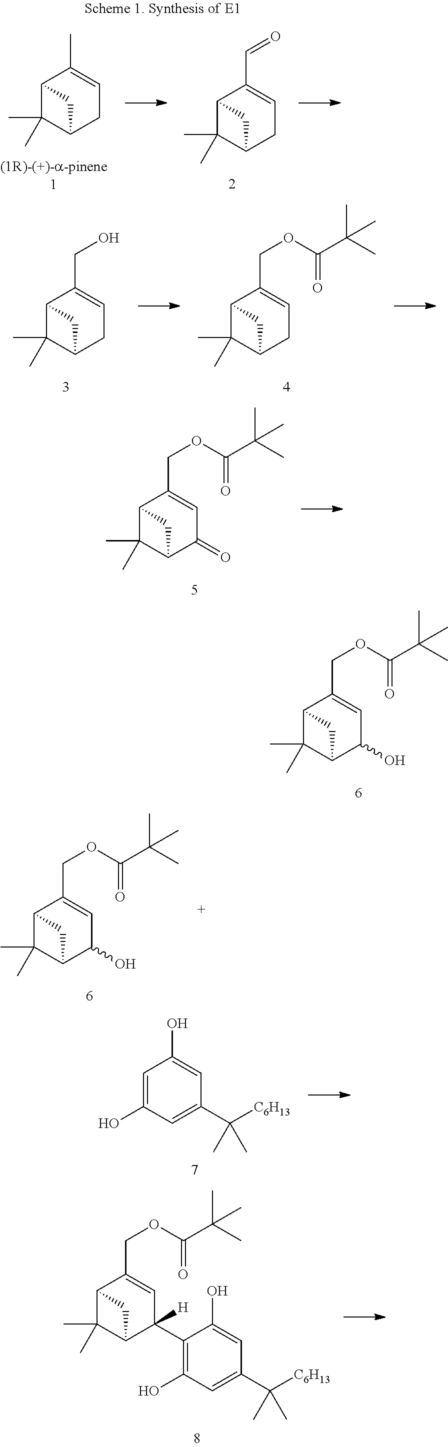

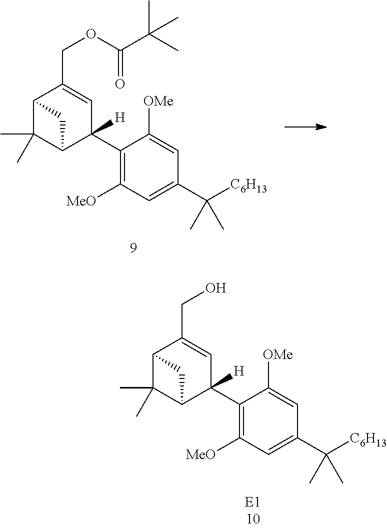

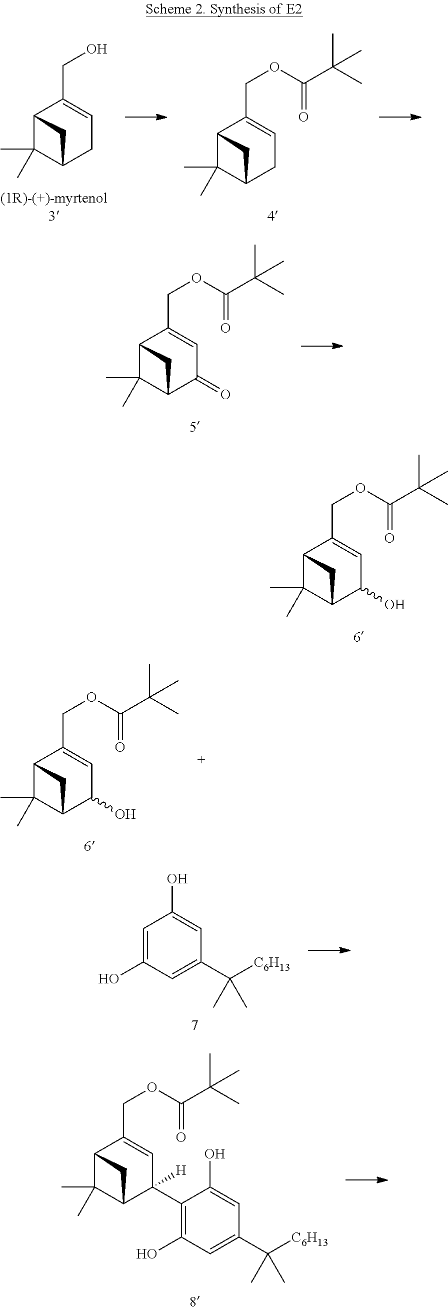

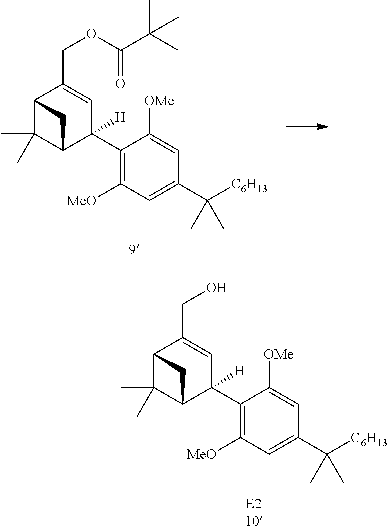

The compound of Formula I (enantiomer E1) can be synthesized via a series of eight steps beginning with (1R)-(+)-α-pinene as starting material. Briefly, the methyl carbon at position C2 of (1R)-(+)-α-pinene can be oxidized to produce myrtenal, followed by a reduction to (+)-myrtenol. Then, the alcohol group of (+)-myrtenol can be protected with a pivaloyl group. The protected (+)-myrtenol can be further oxidized at the methylene carbon (C4 position) and reduced to obtain 4-hydroxy-myrtenyl pivalate. Next, the 4-hydroxy-myrtenyl pivalate can be condensed with 5-(1,1-dimethylheptyl)-resorcinol affording (2-[2,6-dihydroxy-4-(2-methyloctan-2-yl)phenyl]-7,7-dimethyl-4-bicyclo[3.1.1]hept-3-enyl]pivalate). In the following step, the alcohol groups at C2 and C6 positions of the resorcinol moiety can be methylated into methoxy groups. In the final step, the pivalate protecting group can be removed through reduction of (2-[2,6-dimethoxy-4-(2-methyloctan-2-yl)phenyl]-7,7-dimethyl-4-bicyclo[3.1.1]hept-3-enyl]pivalate) to afford the desired product (E1, the compound of Formula I). Enantiomerically pure material can be obtained by chiral separation of the desired component.

The compound of Formula II (enantiomer E2), can be synthesized in 6 steps from (1R)-(−)-myrtenol. Briefly, the alcohol group at the C2 position of (1R)-(−)-myrtenol can be protected using a pivaloyl protecting group. The protected (1R)-(−)-myrtenol can then be oxidized at its C4 position to generate 4-oxo-myrtenyl pivalate which is subsequently reduced into 4-hydroxy-myrtenyl pivalate. The (1R)-(−)-4-hydroxy-myrtenyl pivalate can then be condensed with 5-(1,1-dimethylheptyl)-resorcinol to give (2-[2,6-dihydroxy-4-(2-methyloctan-2-yl)phenyl]-7,7-dimethyl-4-bicyclo[3.1.1]hept-3-enyl]pivalate). In the final two steps, the alcohols at C2 and C6 positions can be methylated and the pivalate protecting group can be removed via reduction producing the compound of Formula II (enantiomer E2). Enantiomerically pure material can be obtained by chiral separation of the desired component.

Compound 3′ of Formula II can also be synthesized beginning with (1S)-(+)-α-pinene as starting material in a two-step synthesis. Briefly, the methyl carbon at position C2 of (1 S)-(+)-α-pinene can be oxidized to produce myrtenal, followed by a reduction to (+)-myrtenol. The (+)-myrtenol can then be used as described to generate the compound of Formula 2 (E2).

E1 and E2 may be enantiomerically purified at the end of their respective synthesis routes via a method known to one skilled in the art (e.g., a chiral HPLC, SFC). Once enantiomerically pure materials are achieved, they can be combined in the desired amounts to achieve the desired ratio of E1 to E2, e.g., 99:1.

Alternatively, the starting materials of E1 and E2 or the synthetic intermediates may be enantiomerically purified. The E1/E2 ratio of the contemplated composition may then be controlled by mixing known amounts of enantiomerically pure E1 and E2 and confirming the ratio by a chiral HPLC.

Alternatively, a composition containing the compounds of Formula I and Formula II can be synthesized via a series of three steps beginning with 4-hydroxy-myrtenyl pivalate as starting material. Briefly, (1S)-(+)4-hydroxy-myrtenyl pivalate is mixed with (1R)-(−)-4-hydroxy-myrtenyl pivalate and the mixture condensed with 5-(1,1-dimethylheptyl)-resorcinol to produce (2-[2,6-dihydroxy-4-(2-methyloctan-2-yl)phenyl]-7,7-dimethyl-4-bicyclo[3.1.1]hept-3-enyl]pivalate). In a next step, the alcohol groups at C2 and C6 positions of the resorcinol moiety are methylated into methoxy groups. In the final step, the pivalate protecting group is removed through reduction of (2-[2,6-dimethoxy-4-(2-methyloctan-2-yl)phenyl]-7,7-dimethyl-4-bicyclo[3.1.1]hept-3-enyl]pivalate) to produce the desired composition.

Similarly, a composition containing compounds of Formula I and Formula II can be synthesized by mixing the equivalent products of any one step of Scheme 1 and 2, at a desired intra-process stage, and then completing the remaining synthetic steps of the synthesis via a single reaction scheme.

### IV. Pharmaceutical Formulations and Administration

As described in hereinbelow, pharmaceutical compositions of containing a CB2RA Composition described herein may be specially formulated for administration in solid or liquid form, including those adapted for the following: (1) oral administration, for example, solutions or suspensions (e.g., aqueous or non-aqueous solutions or suspensions), tablets (e.g., those targeted for buccal, sublingual, and/or systemic absorption or topical local absorption), boluses, powders, including powders for pulmonary delivery, granules, pastes for application to the tongue; (2) parenteral administration by, for example, intravenous, subcutaneous, intramuscular, or epidural injection as, for example, a sterile solution or suspension, or sustained- or otherwise modified release formulation; (3) topical application, for example, as a cream, ointment, a powder or a controlled-release patch, or implant, or spray applied to the skin or inhaled; (4) intravaginally or intrarectally, for example, as a pessary, cream or foam; (5) sublingually; (6) ocularly; (7) transdermally; or (8) nasally. In certain embodiments, the pharmaceutical compositions may be administered to a subject in need thereof orally. In other embodiments, the pharmaceutical compositions may be administered to a subject in need thereof intravenously or subcutaneously or intraperitoneally or intra-ocularly or periocularly. In certain embodiments, the pharmaceutical compositions may be administered to a subject in need thereof topically. In certain embodiments, the pharmaceutical compositions may be administered to a subject in need thereof intranasally.

Irrespective of the enantiomeric excess or weight ratio of the enantiomers, the compositions described herein containing E1 and E2 typically have an aqueous solubility of less than 0.5 mg/L. As a result, the compositions preferably are formulated so as to increase aqueous drug concentration, especially if intravenous, subcutaneous or ocular dosing is to be achieved. It is contemplated that formulations containing a certain polymer technologies, for example, PVP-PLA-based polymers, can be used to enhance the solubility up to at least 5 g/L, which represents a solubility increase of over four orders of magnitude.

It is understood that the pharmaceutical compositions can be formulated depending upon the mode of delivery desired. However, it is contemplated that the pharmaceutical composition is formulated with one or more pharmaceutically acceptable excipients, which can include, for example, solubilizing polymer (for example, an amphipathic polymer including a non-ionic polymer), a buffer or buffering agent, a salt, or a combination thereof. Other excipients that may be used in a pharmaceutical composition include complex sugars (for example dextrose, cyclodextrin, fructose, glucose, trehalose and mannitol, cellulose derivatives, starches and starch derivatives), oils and waxes (for example mineral oil, cottonseed oil and soybean oil), stabilizing agents (for example glycine, histidine or lecithin), viscosity enhancing agents (for example alginates, carbomers, chitosan, xanthan gum, polyvinyl alcohols and hyaluronic acid), stabilizing agents (for example ascorbic acid, cysteine, glutamate and alpha tocopherol) and preservatives (for example benzalkonium chloride, benzyl alcohol and sodium benzoate), emulsifying agents, coloring agents, release agents, coating agents, sweetening agents, and lubricants.

An exemplary solubilizing polymer may be a polyvinylpyrrolidone-polylactic acid (PVP-PLA) copolymer, for example, as described in International Publication No. WO2018176158A1, the teachings of which are incorporated by reference herein in entirely. The PVP-PLA polymer comprises structure of Formula III:

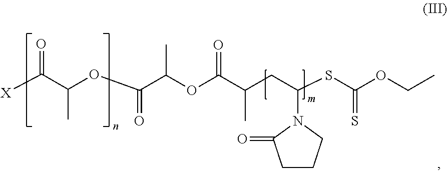

where x is an initiator alcohol having a boiling point greater than 145° C., n is, on average, from 20 and 40, and m is, on average, from 10 and 40, wherein the block copolymers have a number average molecular weight (Mn) of at least 3,000 Da. An “initiator alcohol” is understood to mean a species having a hydroxyl group capable of serving a substrate for polymerization, in this case of poly (D,L lactide) (PLA).

The initiator alcohol can be selected from the group consisting of: 1-hexanol; 1-heptanol; diethylene glycol monoethyl ether; diethylene glycol mono methyl ether; triethylene glycol mono methyl ether; tetraethylene glycol mono methyl ether; oligo-ethylene glycol mono methyl ethers of formula IV

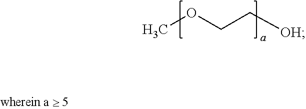

oligo-ethylene glycol mono ethyl ethers of formula V

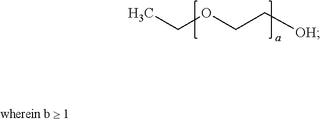

and mixtures thereof. It is contemplated that x can be, for example, diethylene glycol mono ethyl ether (DEGMEE). In certain embodiments, the block copolymers have a Mn of less than 12,000 Da, 11,000 Da, 10,000 Da, 9,000 Da, 8000 Da, 7000 Da, or 6000 Da. In certain embodiments, the block copolymers have a Mn of less than 7,000 Da. In certain embodiments, the block copolymers have a Mn of greater than 4,000 Da, 5,000 Da, or 6,000 Da.

In certain embodiments the pharmaceutical composition may comprise from about 5% to about 95%, or from 30% to about 90%, or from about 60% to about 85% or from about 70% to about 80%. by weight of a block copolymer.

In certain embodiments, the block copolymers are capable of forming microparticles containing the composition of the invention, wherein the microparticles are suitable for administration to a subject. The microparticles nanoparticles, may have an average particle size less than 1000 μm, 500 μm, 100 μm, 75 μm, 50 μm, 25 μm, 10 μm or 1 μm. The microparticles may be sized to allow subcutaneous delivery of the composition of the invention or may be sized to allow delivery of the composition of the invention to the eye including by intra-ocular, peri-ocular or supra-ocular routes. For example, microparticles may be size greater than 250 nm (Lengyel, et al. (2019) SCIENTIA PHARMACEUTICA, 87(3): 20-31).

In certain embodiments, the block copolymers are capable of forming nanoparticles containing a CR2RA Composition, wherein the nanoparticles are suitable for administration to a subject. The nanoparticles may have an average particle size less than 500 nm, 400 nm, 300 nm, 200 nm, 100 nm, 75 nm, 50 nm, or 25 nm. The nanoparticles may be sized to avoid or reduce renal excretion. For example, nanoparticles may have an average particle size greater than 12 nm.

In addition, a CB2RA Composition may be formulated in micelles to produce a micellar composition. For example, the micelles can be produced from amphipathic polymers (e.g., PVP-PLA block copolymers, which contain both hydrophobic regions (PLA groups) as well as hydrophilic regions (PVP groups). A typical micelle in aqueous solution forms with the hydrophilic regions of the polymer in contact with surrounding solvent, sequestering the hydrophobic regions in the micelle core. Micelles can be present in solution or in dry form (e.g., a cake or powder) that may be administered to a subject or rehydrated to produce a solution that may be administered to a subject. For example, micelles include a dried form of a previously liquid colloidal composition of micelles, wherein some elements of a micellar structure are retained in dried form, or wherein the dried form readily reforms micelles upon hydration. When dried, the micelles may be rehydrated in a solvent, for example water (e.g., WFI), bacteriostatic water, a dextrose solution (for example a 5% dextrose solution), Ringer's solution, Ringer's lactate solution or saline. In certain embodiments the formulations may contain a preservative or anti-microbial agent such as benzylkonium chloride, benzyl alcohol, sodium benzoate and phenol.

In certain embodiments, the formulations (for example, the micellar formulations) may be an essentially clear liquid, for example, free of visible particulates. In certain embodiments, solution can have an optical transmittance indicative of the general clearness of a solution. The optical transmittance may be, for example, greater than 70%, 80%, 90%, 95%, 96%, 97%, or 98%.

It is contemplated that, in the appropriate situations, the pharmaceutical compositions may comprise a hydrogel such as a thermoplastic hydrogel, or a cross-linked hydrogel, or a tunable hydrogel, or an adhesive hydrogel or an implantable hydrogel (Jainyu (2016) NAT. REV. MATER., 1(12): 1-38).

It is contemplated that, under certain circumstances, the hydrogel may be a starch-based hydrogel and preferably a cross-linked high amylose hydrogel, for example, as described in Canadian Patent No. 3034722.

It is contemplated that the hydrogel may contain polymers including cross-linkable polymers.

It is contemplated that the pharmaceutical compositions may display immediate release or modified release characteristics where modified release includes accelerated release, burst release, delayed release, extended release, single phase, dual phase, pulsed or multi-phase release.

It is contemplated that the pharmaceutical compositions can comprise a buffer or buffering agent. Exemplary buffering agents include, for example, maleic acid, malic acid, meglumine, methionine, potassium chloride, potassium hydroxide, sodium carbonate, sodium bicarbonate, sodium chloride, sodium hydroxide, sodium lactate, sodium phosphate, potassium phosphate, phosphate acid, sulfuric acid, sodium acetate, ammonium acetate, acetic acid, tromethamine (Tris), sodium citrate/citric acid. In one embodiment the buffer or buffering agent is a phosphate buffer. In certain embodiments, the buffer or buffering agent is a Tris buffer. Alternatively, or in addition, the pharmaceutical compositions can include one or more salts, for example, to produce an isotonic solution. Exemplary salts include, for example, an ammonium salt, or a calcium salt. In certain embodiments, the salt may be a sodium salt, or a potassium salt.

It is contemplated that the pharmaceutical compositions may include a bulking agent. Exemplary bulking agents include dextrin dextrose, fructose, gelatin, glucose, lactose, maltose, mannitol, polyvinylpyrrolidone, sucrose, sorbitol, trehalose and raffinose.

It is contemplated that the pharmaceutical compositions may include a solvent or vehicle. Exemplary solvents and vehicles include, for example, water, saline, ethanol, cottonseed oil, soybean oil, dimethyl sulfoxide, dimethylacetamide, ethyl oleate, glycerin, glycofurol, mineral oil, monoethanolamine, polyethylene glycol, propylene glycol and methylpyrrolidone.

It is contemplated that the pharmaceutical compositions may include a solubility enhancer. Exemplary solubility enhancers include oleoyl polyoxyl-6 glyderides such as Labrafil® M1944 CS (Gattefossé Inc.), castor oil, caprylic acid, a-cyclodextrin, g-cyclodextrin, b-cyclodextrin, disodium edetate, gelucire 44/14 (Gattefossé Inc.), hydroxypropyl betadex, macrogol 15 hydroxystearate, medium-chain triglycerides, poly(L-lactide), poly(DL-lactide), poly(lactide-co-glycolide or PLGA), a poloxamer, polyoxyethylene castor oil derivatives, polyoxyethylene sorbitan fatty acid esters, povidone, and triolein.

It is contemplated that the pharmaceutical compositions may include a viscosity controlling or gelling agent. Exemplary viscosity controlling or geling agents include alginic acid, a sodium alginate, carboxymethylcellulose sodium, carbomers, chitosan, hyaluronic acid, hypromellose, hydroxypropyl cellulose, polyvinyl alcohol, xanthan gum and xyloglucan.

It is contemplated that the pharmaceutical compositions may include an antioxidant. Exemplary antioxidants include ascorbate, argon, sodium bisulfite, butylated hydroxy anisol, butylated hydroxy toluene, cysteine, sodium dithionite, gentisic acid, glutamate, potassium metabisulfite, thioglycerol, nitrogen, sodium sulfite and alpha-tocopherol.

It is contemplated that the pharmaceutical compositions may include a stabilizing agent. Exemplary stabilizing agents include albumin, diethanolamine, glycine, histidine and lecithin.

It is contemplated that the pharmaceutical compositions may include a release controlling polymer. Exemplary release controlling polymers include, for example, polyethylene oxide, polyvinyl pyrrolidone-polylactide copolymers, polyvinylpyrrolidone, pullulan, pectin, chitosan, sodium alginate, carrageenan, gelatin, methyl cellulose, carboxymethylcellulose sodium, crosslinked carboxymethylcellulose sodium, crosslinked hydroxypropylcellulose, cross-linked starch, cross linked high amylose starch, hydroxypropylmethylcellulose, carboxymethyl starch, polymethacrylate, polyvinylpyrrolidone, polyvinyl alcohols, polyethylene glycols, or potassium methacrylate-divinyl benzene copolymer and mixtures thereof.

It is contemplated that the pharmaceutical composition may include a release accelerating agent. Exemplary release accelerating agents include starches and cross-linked starches and starch glycolates, cross-linked celluloses, calcium silicate and polyvinyl pyrrolidone.

In certain embodiments, the composition may comprises from about 0.25% (w/w) to about 80% (w/w), from about 0.5% (w/w) to about 60% (w/w), or from about 0.75% (w/w) to about 30% or about 1% (w/w) to about 20% (w/w) of the CB2RA Composition. In certain embodiments, the composition may comprise from about 0.5% (w/w) to about 10% (w/w), from about 0.5% (w/w) to about 20% (w/w), or from about 0.75% (w/w) to about 10% (w/w) or about 1% to about 5% (w/w) of the CB2RA Composition.

In certain embodiments, the composition may comprise from about 1% to about 20%, from about 5% to about 15%, about 10%, about 11%, about 12%, about 13%, about 14%, about 15%, about 16%, about 17%, about 18%, about 19%, about 20% by weight the buffer. In certain embodiments, the pharmaceutical composition, when in liquid form, may have a pH from about 4 to about 9, from about 6 to about 8, or from about 6.5 to about 7.5. In other embodiments, the composition may have a pH of about 6, about 6.5, about 7, about 7.5, or about 8.

In certain embodiments, a pharmaceutical composition described herein, when in liquid, gel, ointment or other semi-solid form, may have a viscosity range from about 0.2 mPas to about 80,000 mPas, from about 0.5 mPas to about 70,000 mPas, from about 1 mPas to about 60,000 mPas, from about 10 mPas to about 50,000 mPas, from about 50 mPas to about 50,000 mPas, from about 100 mPas to about 50,000 mPas.

In certain embodiments, a pharmaceutical composition described herein, when in the form of a micellar solution may comprise particles having a particle size (Z.av) of 12-50 nm, 20-40 nm, 10-20 nm, 20-40 nm, 20-35 nm, 20 nm-30 nm, 25 nm-40 nm, 25 nm-35 nm, about 20 nm, about 25 nm, about 30 nm, about 35 nm, about 40 nm, about 50 nm. In certain embodiments, the particles may have a polydispersity index (PDi) of from about 0.05 to about 0.15, from about 0.1 to about 0.15, from about 0.05 to about 0.1 about 0.05, about 0.10, about 0.11, about 0.12, about 0.13, about 0.14, or about 0.15.

It is contemplated that the formulations may further include wetting agents, emulsifiers, lubricants, coloring agents, release agents, coating agents, sweetening, flavoring and preservatives, and antioxidants. Exemplary wetting agents include, for example, benzalkonium chloride, benzethonium chloride, cetylpyridinium chloride monohydrate, poloxamer 188, poloxamer 407, polyoxyl 40 stearate Type II, polysorbate 20, polysorbate 40. Exemplary emulsifiers include, for example, acacia, carbomer copolymer, carbomer interpolymer, cholesterol, coconut oil, diethylene glycol, stearates, ethylene glycol stearates, glyceryl distearate, glyceryl monolinoleate, glyceryl monooleate, glyceryl monostearate, lanolin alcohols, lecithin mono- and di-glycerides, poloxamer, polyoxyethylene stearate, polyoxyl 10 oleyl ether, polyoxyl 20 cetostearyl ether, polyoxyl 35, castor oil, polyoxyl 40, hydrogenated castor oil, polyoxyl 40 stearate, polyoxyl lauryl ether, polyoxyl stearyl ether, polysorbate 20, polysorbate 40, polysorbate 60, polysorbate 80, propylene glycol monostearate, sodium cetostearyl sulfate, sodium lauryl sulfate, sodium stearate, sorbitan monolaurate, sorbitan monooleate, sorbitan monopalmitate, sorbitan monostearate, sorbitan sesquioleate, sorbitan trioleate stearic acid and wax (emulsifying).

Exemplary lubricants include, for example, calcium silicate, calcium stearate, Hypromellose, magnesium stearate, mineral oil, polyethylene glycol, polyoxyl stearate, polysorbate, polyvinyl alcohol, colloidal silicon dioxide, sodium lauryl sulphate, sodium stearyl fumarate, sorbitan monoleate, sorbitan monoplamitate, sorbitan monostearate, sorbitan sesquioleate, stearic acid and Talc. Exemplary release controlling agents include, for example, polyethylene oxide, polyvinyl pyrrolidone-polylactide copolymers, polyvinylpyrrolidone, pullulan, pectin, chitosan, sodium alginate, carrageenan, gelatin, methyl cellulose, carboxymethylcellulose sodium, crosslinked carboxymethylcellulose sodium, crosslinked hydroxypropylcellulose, cross-linked starch, cross linked high amylose starch, hydroxypropylmethylcellulose, carboxymethyl starch, polymethacrylate, polyvinylpyrrolidone, polyvinyl alcohols, polyethylene glycols, or potassium methacrylate-divinyl benzene copolymer and mixtures thereof. Exemplary coating agents include, for example ethyl cellulose, gelatin, hydroxypropyl cellulose, hypromellose, methylcellulose, polyethylene glycol, polyvinyl alcohol, polyvinyl pyrrolidone, methacrylic acid, hydroxyethylmethylcellulose, ethyl cellulose, polyvinyl acetate phthalate, cellulose acetate phthalate, hydroxy propyl methyl cellulose phthalate, maleic anhydride copolymers, including poly (methyl vinyl ether/maleic anhydride) and hydroxypropylmethylcellulose.

Exemplary preservatives include, for example, methyl paraben ethyl paraben, propyl paraben, butyl paraben, benzyl alcohol, chlorobutanol, phenol, meta cresol, chloro cresol, benzoic acid, sorbic acid, thiomersal, phenylmercuric nitrate, bronopol, propylene glycol, benzylkonium chloride and benzethonium chloride. Exemplary antioxidants include, for example, (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In one embodiment, the pharmaceutical composition comprises up to 15% wt/wt of an impurity which has a structure of Formula B:

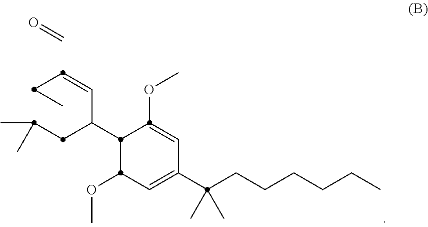

In one embodiment, the pharmaceutical composition comprises up 14% wt/wt of the impurity. In one embodiment, the pharmaceutical composition comprises up 13% wt/wt of the impurity. In one embodiment, the pharmaceutical composition comprises up 12% wt/wt of the impurity. In one embodiment, the pharmaceutical composition comprises up 11% wt/wt of the impurity. In one embodiment, the pharmaceutical composition comprises up 10% wt/wt of the impurity. In one embodiment, the pharmaceutical composition comprises up 9% wt/wt of the impurity. In one embodiment, the pharmaceutical composition comprises up 8% wt/wt of the impurity. In one embodiment, the pharmaceutical composition comprises up 7% wt/wt of the impurity. In one embodiment, the pharmaceutical composition comprises up 6% wt/wt of the impurity. In one embodiment, the pharmaceutical composition comprises up 5% wt/wt of the impurity. In one embodiment, the pharmaceutical composition comprises up 4% wt/wt of the impurity. In one embodiment, the pharmaceutical composition comprises up 3% wt/wt of the impurity. In one embodiment, the pharmaceutical composition comprises up 2% wt/wt of the impurity. In one embodiment, the pharmaceutical composition comprises up 1% wt/wt of the impurity. In one embodiment, the pharmaceutical composition comprises up 0.5% wt/wt of the impurity. In one embodiment, the pharmaceutical composition comprises up 0.1% wt/wt of the impurity. In one embodiment, the pharmaceutical composition comprises up 0.05% wt/wt of the impurity. In one embodiment, the pharmaceutical composition comprises up 0.01% wt/wt of the impurity. In one embodiment, the pharmaceutical composition comprises up 0.005% wt/wt of the impurity. In one embodiment, the pharmaceutical composition comprises up 0.001% wt/wt of the impurity.

In some embodiments, the amount of the impurity measured above is following incubation at 40° C. In some embodiments, the amount of the impurity measured above is following incubation at 40° C. for 1 month. In some embodiments, the amount of the impurity measured above is following incubation at 40° C. for 2 months. In some embodiments, the amount of the impurity measured above is following incubation at 40° C. for 3 months. In some embodiments, the amount of the impurity measured above is following incubation at 40° C. for 6 months.

In some embodiments, the amount of the impurity measured above is following incubation at 65° C. In some embodiments, the amount of the impurity measured above is following incubation at 65° C. for 24 hours. In some embodiments, the amount of the impurity measured above is following incubation at 65° C. for 48 hours.

In some embodiments, the amount of the impurity measured above is following incubation at 80° C. In some embodiments, the amount of the impurity measured above is following incubation at 80° C. for 24 hours. In some embodiments, the amount of the impurity measured above is following incubation at 80° C. for 48 hours.

In one embodiment, the pharmaceutical composition further comprises an antioxidant for reducing formation of the impurity.

In one embodiment, the antioxidant is selected from the group consisting of ascorbic acid, butylated hydroxytoluene, sesamol, guaiac resin, methionine, citric acid, tartaric acid, phosphoric acid, thiol derivatives, potassium metabisulphite, ascorbyl palmitate, calcium stearate, propyl gallate, sodium thiosulphate, glutathione, dihydroxybenzoic acid, benzoic acid, urate and uric acid, sorbic acid, sodium benzoate, EDTA, sodium bisulphite, vitamin E, cysteine hydrochloride, sorbitol, butylated hydroxyanisol, and mixtures thereof.

In one embodiment, the antioxidant is selected from the group consisting of EDTA, sodium bisulphite, vitamin E, cysteine hydrochloride, sorbitol, butylated hydroxyanisol, and mixtures thereof.

In one embodiment, the antioxidant comprises butylated hydroxyanisol.

In one embodiment, the pharmaceutical composition is essentially free of oxygen.

In one embodiment, the pharmaceutical composition is free of oxygen.

In one embodiment, the pharmaceutical composition is stored under an inert gas.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. The pharmaceutical composition may comprise for example, 0.1 mg to 1 mg, 5 mg to 50 mg, 50 mg to 500 mg or 500 mg to 1000 mg of the active ingredient. To the extent that the composition of the invention is in liquid form, the concentration of the active ingredient may be from about 0.05 mg/mL to about 0.5 mg/mL, from about 0.1 mg/mL to about 5 mg/mL from about 0.5 mg/mL to about 5 mg/mL, from about 1 mg/mL to 5 mg/mL, from about 2 mg/mL to 5 mg/mL, about 0.5 mg/mL, about 1 mg/mL, about 2 mg/mL, about 3 mg/mL, about 4 mg/mL, about 5 mg/mL, about 10 mg/mL, about 20 mg/mL or about 30 mg/mL.

The present disclosure demonstrates that by formulating a CB2RA Composition in a micellar composition, comprising di-block polymers of polyvinylpyrrolidone and polylactide of defined molecular weight and block to block ratio (PVP-PLA) that the apparent aqueous solubility of the CB2RA Composition can be increased many thousand fold, from milligram to gram per litre levels, generating a solution of viscosity, pH, and the concentration of the CB2RA Composition sufficient to allow simple parenteral administration to mammals and/or simple topical administration to mammals.

In liquid dosage forms for topical administration such as a cream, ointment, spray or an eye drop, the active ingredient may be mixed with one or more pharmaceutically acceptable polymers such as; (1) an amphiphilic polymer, including PVP-PLA; (2) a solubilizing agent such as oleoyl polyoxyl-6 glyderides such as Labrafil® M1944 CS (Gattefossé Inc.), a-cyclodextrin, g-cyclodextrin, b-cyclodextrin, disodium edetate, hydroxypropyl betadex, macrogol 15 hydroxystearate, medium-chain triglycerides, poly(L-lactide), poly(DL-lactide), poly(lactide-co-glycolide or PLGA), a poloxamer, polyoxyethylene castor oil derivatives, polyoxyethylene sorbitan fatty acid esters, povidone, and triolein or mixtures thereof; (3) a buffer, such as a phosphate or citrate or bicarbonate or Tris buffer (4) a preservative or antioxidant such as ascorbic acid or cysteine hydrochloride and; (5) a preservative such as benzylkonium chloride or benzethonium chloride and (6) a solvent such as water, saline, ethanol, cottonseed oil, soybean oil, dimethyl sulfoxide, dimethylacetamide, ethyl oleate, glycerin, glycofurol, mineral oil, monoethanolamine, polyethylene glycol, propylene glycol and methylpyrrolidone, of mixtures thereof.

In solid dosage forms of the invention for oral administration (capsules, tablets, pills, dragees, powders, granules, trouches and the like), the active ingredient may be mixed with one or more pharmaceutically-acceptable carriers, such as sodium citrate or dicalcium phosphate, and/or any of the following: (1) fillers or release controller agents or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres and or nanospheres or other nanoparticles. They may be formulated for rapid release. They may be sterilized by, for example, filtration through a bacteria-retaining filter, or irradiation, or autoclaving, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Dosage forms for the topical or transdermal administration of a compound of this invention include ointments, pastes, creams, lotions, gels, solutions, powders, sprays, patches and inhalants. The active compound may be mixed under sterile conditions with a pharmaceutically-acceptable carrier, and with any preservatives, buffers, or propellants which may be required.

The ointments, pastes, creams and gels may contain, in addition to the active ingredients(s), excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the active ingredient(s) invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the advantage of providing controlled delivery of a CB2RA Composition to a subject. Such dosage forms can be made by dissolving or dispersing the active ingredient(s) in the proper medium. Absorption enhancers can also be used to increase the flux of the compound across the skin. The rate of such flux can be controlled by either providing a rate controlling membrane or dispersing the compound in a polymer matrix or gel.

Injectable depot forms can be made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) poly(anhydrides) and complex carbohydrates. Depot injectable formulations can be prepared by entrapping the drug in liposomes or microemulsions which are compatible with body tissue.

Implantable depot forms for a compound of this invention may be in the form of a surgical mesh, a silicone-based implant, a polyethylene-based implant, a titanium based implant, a polyurethane foam implant, a polylactic acid implant, a 3D printed biomaterial and the like. Implantable depots forms may also be in the form of a hydrogel, whether a solid or liquid or semi-solid hydrogel including biopolymer and synthetic polymer hydrogels, thermoplastic hydrogels, thermoplastic gels, cross linked or cross-linkable hydrogels (including collagen containing hydrogels or collagen derived peptide containing hydrogels). Hydrogels may and other implantable devices may comprise ethylene vinyl acetate and acrylates. Implants may include biodegradable implants and non-biodegradable implants.

Actual dosage levels of the CB2RA Compositions may be varied so as to obtain an amount of the active ingredient which is effective to achieve the desired therapeutic response for a particular patient, composition, and mode of administration, without being toxic to the subject. The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

In some embodiments, a physician or veterinarian having ordinary skill in the art can readily determine and prescribe the effective amount of the CB2RA Composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable dose of a CB2RA Composition will be that amount of the composition which is the lowest dose effective to produce a therapeutic effect. Such an effective dose will generally depend upon the factors described above. Preferably, the compounds of the invention are administered at about 0.01 mg/kg to about 200 mg/kg, more preferably at about 0.1 mg/kg to about 100 mg/kg, even more preferably at about 0.5 mg/kg to about 50 mg/kg. When the compositions described herein are co-administered with another agent (e.g., as sensitizing agents, or as a fixed-dose combinations, or as adjuvants), the effective amount may be less than when the agent is used alone.

If desired, an effective dose of the active compounds may be administered daily as two, three, four, five, six or more sub-doses administered separately at appropriate intervals throughout the day, optionally, in unit dosage forms. Under certain circumstances, dosing preferably involves one administration per day via oral administration. If desired, the effective dose of the active compound may be delivered weekly as a single administered dose or biweekly as a single administered dose. If desired the effective dose of the active compound may be delivered monthly as a single administered dose. If desired the effective dose of the active compound may be delivered at intervals of greater than once per month as a single administered dose.

In certain embodiments, a pharmaceutical composition provides a pharmaceutically effective plasma level of the active ingredient(s) within 4, 3, 2, 1 or 0.5 hours of administration. The term “pharmaceutically effective plasma level” is understood to mean an amount of the active ingredient(s) that, when in plasma, is sufficient to achieve desired therapeutic efficacy. This level can vary depending, for example, upon the disease, disorder, and/or symptoms of the disease or disorder, severity of the disease, disorder, and/or symptoms of the disease or disorder, the age, weight, and/or health of the subject to be treated.

In certain embodiments, the pharmaceutical composition provides a pharmaceutically effective plasma level of the active ingredient(s) within 20, 15, 10, 9, 8, 7, 6, 5, 4, 3 or 2 minutes of administration. In certain embodiments a pharmaceutically effective plasma level of the active ingredient(s) is maintained for at least 2, 3, 4, 5, 6, 7, 8, 9, 10, 11, 13 or 24 hours. In certain embodiments a pharmaceutically effective plasma level of the active ingredient(s) is maintained for at least 2, 3, 4, 5, 6 or 7 days. In certain embodiments a pharmaceutically effective plasma level of the active ingredient(s) is maintained for at least 2, 3, 4, or 5 weeks. In certain embodiments a pharmaceutically effective plasma level of the active ingredient(s) is maintained for at least 2, 3, 4, 5, 6, 7, 8, 9, 10, 11 or 12 months.

Exemplary pharmaceutical compositions are set forth in Examples 10-24.

Example 10 provides an exemplary lyophilized cake of a CB2RA Composition with PCP-PLA and trehalose that can be used, for example, in a tablet of capsule or reconstituted for injection or a topical administration. Example 11 provides an exemplary spray dried composition of the composition of Example 10 that can be used, for example, in a tablet or capsule, a powder of inhalation. Example 12 provides an exemplary lyophilized cake of a CB2RA Composition with cyclodextrin that can be used, for example, in a tablet of capsule or reconstituted for injection or a topical administration. Examples 13-14 provide exemplary aqueous formulations for topical application, for example, for topical application to the eye. Example 15 provides an exemplary gel formulation for topical application, for example, topical application to the eye. Example 16 provides an exemplary ointment for topical application, for example, topical application to the eye. Examples 17 and 18 provide exemplary oral controlled release dosage forms. Examples 19 and 20 provide exemplary oral immediate release formulations. Example 21 provides an exemplary oral, immediate and delayed release formulation. Examples 22 and 23 provide exemplary powders for incorporation into capsules. Example 24 provide an exemplary combination product containing a combination of a CB2RA Composition and celecoxib in an oral, immediate release dosage form. It is contemplated that other dosage forms can be produced depending upon the intended mode of delivery, dosage and use of the CB2RA Composition.

### V. Combination Products

It is understood the pharmaceutical compositions described herein can be administered either alone or together with a pharmaceutically effective, or sub-pharmaceutically effective, amount of a nonsteroidal anti-inflammatory drug (NSAID) and/or a steroid. It is contemplated that the additional agents can be administered (i) separately, for example, via a separate dosage form that is administered to the subject prior to, simultaneous with, or after the composition of the invention, or (ii) in a unitary dosage form that administers the additional agent in combination with composition of the invention.

It has been discovered that the weight ratio, or degree of enantiomeric excess, of E1 to E2, of a CB2RA Composition described herein controls the level of analgesic and/or anti-inflammatory effect exhibited by the compound or composition. In addition, it has been discovered that there is a defined range of weight ratios of E1 to E2 where their combination provides a beneficial, more potent, or more efficacious analgesic and/or anti-inflammatory effect. An exemplary weight ratio range has been established for E1 to E2 of about 99:1 or 99:1.

As the CB2RA Compositions described herein possess a mode of action that is different to those of commonly used analgesics, anaesthetics and anti-inflammatory agents for example opioid narcotic analgesics (for example, oxycodone and its salts), gabapentinoid and GABA receptor mediated agents (for example, pregabalin and its salts or propofol), tricyclic agents (for example, amytryptaline and its salts) cyclooxygenase inhibitors (for example, ketorolac and its salts or celecoxib) and ion channel blocking agents (for example, bupivacaine) the CB2RA Compositions may be used are part of a multi-modal treatment regimen for a subject.

It has been also demonstrated (see, Example 8) that the ‘ceiling-dose’ analgesic effect of NSAID cyclooxygenase (COX) enzyme inhibitors, can be avoided by co-administration with a CB2RA Composition described herein. Without wishing to be bound by theory, it appears that the different, but potentially complementary mechanisms of action of CB₂ receptor agonism and COX enzyme inhibition can be combined to generate a novel and clinically meaningful synergistic enhancement of analgesic activity. For example, when a CB2RA Composition described herein, at a controlled and potent E1 to E2 ratio, was combined with the COX enzyme inhibitor celecoxib synergy occurs such that the amount of each agent needed to achieve meaningful analgesia and/or anti-inflammatory effect is greatly reduced to an extent greater than that which would occur should the effects of each agent be purely additive in nature. This effect, for example, is especially useful because the composition of the invention can be used (i) alone as a new analgesic having a novel mechanism of action that facilitates opioid-sparing or elimination, or COX enzyme inhibitor-sparing or elimination (ii) as part of a multi-modal treatment regimen to enhance the effect of COX enzyme inhibitors, thereby allowing greater analgesia per total amount of analgesic administered, and greater opioid, or other analgesic-sparing for the same administered amount of COX enzyme inhibitor drug, and (iii) as fixed dose combination drug to be used alone, or in combination with other anti-inflammatory and/or analgesic medicines to achieve a desired therapeutic effect while minimizing the occurrence of deleterious adverse side-effects.

It has been demonstrated for the first time that CB₂R receptor agonists, if co-administered with drugs inhibiting cyclooxygenase enzymes, can act synergistically with those drugs to generate a drug combination of superior potency to each drug alone reaching levels of analgesia comparable to those achieved by opioid analgesics.

In certain embodiments, the invention provides a pharmaceutical composition comprising the CB2RA Composition described herein and a pharmaceutically effective amount of a nonsteroidal anti-inflammatory drug (NSAID). The CB2RA Composition and the NSAID may be formulated into a single dosage form.

In certain embodiments, the invention provides a pharmaceutical composition comprising the CB2RA Composition described herein and a pharmaceutically effective amount of a nonsteroidal anti-inflammatory drug (NSAID) and/or a steroid. The CB2RA Composition and the NSAID and/or the steroid may be formulated into a single dosage form.

In one aspect, the present invention provides a pharmaceutical composition comprising the CB2RA Composition described herein and a pharmaceutically effective amount of a nonsteroidal anti-inflammatory drug (NSAID) and/or a steroid and/or a nerve block agent. The CB2RA Composition and the NSAID and/or steroid and/or a nerve block agent may be formulated into a single dosage form.

In certain embodiments, the invention provides a pharmaceutical composition comprising the CB2RA Composition described herein and a pharmaceutically effective amount of a nonsteroidal anti-inflammatory drug (NSAID) and/or a steroid and/or a nerve block agent and/or an immunomodulatory agent. The CB2RA Composition and the NSAID and/or steroid and/or a nerve block agent and/or immunomodulatory agent may be formulated into a single dosage form.

The NSAID may be, for example, bromofenac aspirin, naproxen, bromfenac, diclofenac, meloxicam, ibuprofen, ketoprofen, tolmetin, indomethacin, sulindac, piroxicam, mefenamic acid, etodolac, nepafenac, flurbiprofen, acetaminophen, bromofenac, ketorolac, celecoxib, etoricoxib, lumiroacoxib, rofecoxib, valdecoxib, parecoxib, acemethacin, dexibuprofen, nimesulide, nabumetone, tiaprofenic acid, lornoxicam, tenoxicam, aceclofenac, proglumethacin, dexketoprofen or oxaprozin. In one embodiment, the NSAID is celecoxib.

The steroid may be a corticosteroid and may be, for example, a corticosteroid, including a glucocorticosteroid. The corticosteroid or glucocorticosteroid may be clobetasol propionate, halobetasol propionate, fluocinonide, diflorasone diacetate, desoximetasone, clocortolone pivalate, mometasone furoate, triamcinolone acetonide, betamethasone valerate, fluticasone propionate, prednicarvate, hydrocortisone probutate, triamcinolone acetonide fluocinolone acetonide, dexamethasone loteprednolloteprednol etabonate, alclometasone dipropionate, desonide or hydrocortisone. The corticosteroid may be, for example, dexamethasone, or loteprednol.

The nerve block agent may be procaine, benzocaine, chloroprocaine, cocaine, cyclomethycaine, dimethocaine, piperocaine, propoxycaine, procaine, proparacaine, tetracaine amethocaine, lidocaine, articaine, bupivacaine, cinchocaine, etidocaine, levobupivacaine, lidocaine, mepivacaine, prilocaine, ropivacaine, trimecaine levobupivacaine, prilocaine, tetracaine, lontocaine, septocaine, ropivacaine, bupivacaine, or mepivacaine, which may optionally include, or be administered with adjuvants such as sodium bicarbonate, midazolam, magnesium, ketamine, dexmedetomidine, verapamil and clonidine.

The immunomodulatory agent may be cylcosporin, lifitegrast, azathioprine, mycophenolate mofetil, methotrexate, leflunomide tacrolimus, sirolimus, cyclophosphamide, chlorambucil, or a tumor necrosis factor inhibitor.

In one aspect, there is provided a pharmaceutical composition for agonizing CB2 receptor activity in a subject, the composition comprising a combination of:
(a) a first compound of Formula I:

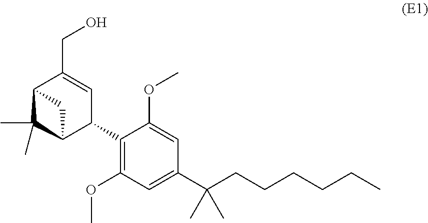 [SMILES: CCCCCCC(C)(C)c1cc(OC)c([C@H]2C=C(CO)[C@H]3C[C@@H]2C3(C)C)c(OC)c1]

or a pharmaceutically acceptable salt thereof; and
(b) a second compound of Formula II:

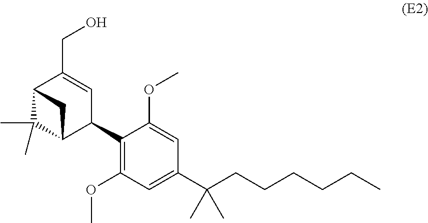 [SMILES: CCCCCCC(C)(C)c1cc(OC)c([C@@H]2C=C(CO)[C@@H]3C[C@H]2C3(C)C)c(OC)c1]

or a pharmaceutically acceptable salt thereof;

In one aspect, there is provided a pharmaceutical composition for agonizing CB2 receptor activity in a subject, the composition comprising a combination of:
(a) a first compound of Formula I:

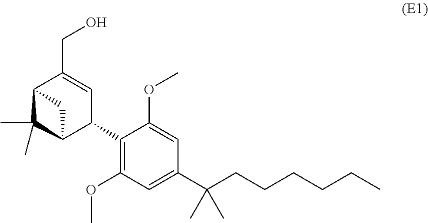 [SMILES: CCCCCCC(C)(C)c1cc(OC)c([C@H]2C=C(CO)[C@H]3C[C@@H]2C3(C)C)c(OC)c1]

or a pharmaceutically acceptable salt thereof; and
(b) a second compound of Formula II:

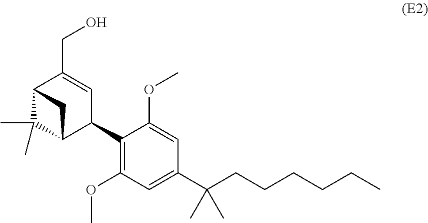 [SMILES: CCCCCCC(C)(C)c1cc(OC)c([C@@H]2C=C(CO)[C@@H]3C[C@H]2C3(C)C)c(OC)c1]

or a pharmaceutically acceptable salt thereof;

wherein the composition comprises the compound of Formula I and the compound of Formula II in a weight ratio of from 99.85:0.15 to 93.5:6.5, and a pharmaceutically acceptable excipient.

In one embodiment, the weight ratio of the compound of Formula I to the compound of Formula II is from 99.8:0.2 to 98.2:1.8.

In one embodiment, the weight ratio of the compound of Formula I to the compound of Formula II is from 98.8:1.2 to 98.4:1.6.

In one embodiment, the weight ratio of the compound of Formula I to the compound of Formula II is from 99.3:0.7 to 98.7:1.3.

In one embodiment, the weight ratio of the compound of Formula I to the compound of Formula II is about 99:1.

In one embodiment, the pharmaceutically acceptable excipient comprises a polymer, a solubilizing agent, a buffer, a salt, a preservative or a combination thereof.

In one embodiment, the polymer is a polyvinylpyrrolidone-polylactic acid (PVP-PLA) copolymer.

In one embodiment, the PVP-PLA copolymer has the structure of Formula III:

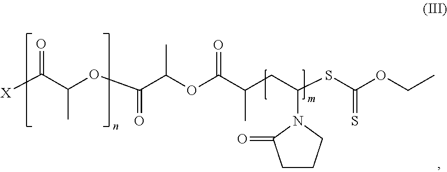

wherein X is an initiator alcohol having a boiling point greater than 145° C., n is, on average, from 20 and 40, and m is, on average, from 10 and 40, wherein the block copolymers have a number average molecular weight (Mn) of at least 3,000 Da.

In one embodiment, the buffer is a phosphate buffer.

In one embodiment, the salt is a sodium salt, or a potassium salt.

In one embodiment, the composition is in the form of a micellar preparation.

In one embodiment, the micellar preparation is in the form of a liquid.

In one embodiment, the micellar preparation is dehydrated into a solid form (e.g., a lyophilized or sprayed dried solid).

In one embodiment, the composition comprises from about 0.25% (w/w) to about 60% (w/w), from about 0.5% (w/w) to about 40% (w/w), or from about 0.75% (w/w) to about 30% or about 1% (w/w) to about 20% (w/w) of the first and second compounds in combination.

In one embodiment, the composition comprises from about 5% to about 99.5%, or from about 5% to 95%, or from 30% to about 90%, or from about 60% to about 85% or from about 70% to about 80%. by weight of the polymer.

In one embodiment, the composition comprises from about 1% to about 20% by weight the buffer.

In one embodiment, the composition further comprises an emulsifying agent, an antioxidant, a controlled release agent, a lubricant, or a flavoring agent.

In one embodiment, the solid form has been rehydrated in a solvent to produce a micellar solution.

In one embodiment, the solvent is water (e.g., WFI), an alcohol, a dextrose solution (e.g. a 5% dextrose solution) or saline.

In one embodiment, the concentration of the first and second compounds in combination is from about 0.1 mg/mL to about 50 mg/mL.

In one embodiment, the concentration of the first and second compounds in combination is from about 0.5 mg/mL to about 15 mg/mL.

In one embodiment, the composition has a pH from about 5 to about 9, from about 6 to about 8, or from about 6.5 to about 7.5 and/or has a viscosity in the range from about from about 0.2 mPas to about 80,000 mPas.

In one embodiment, the micellar solution comprises particles having a particle size (Z.av) of 5 nm-100 nm, 10-50 nm, 12050 nm, 15-45 nm, or 20-40 nm.

In one embodiment, the particles have a polydispersity index (PDi) of from about 0.05 to about 0.15.

In one embodiment, the particles have a polydispersity index (PDi) of from about 0.05 to about 0.2.

In one embodiment, the composition further comprises from 0.015% to 1.5% of a third or fourth compound.

In one embodiment, the composition further comprises from 0.01% to 30% of a third or fourth compound.

In one aspect, there is provided a pharmaceutical composition comprising the pharmaceutical composition as defined herein and a pharmaceutically effective amount of a nonsteroidal anti-inflammatory drug (NSAID) and/or a steroid.

In one embodiment, the NSAID is bromofenac, nepafenac, aspirin, naproxen, diclofenac, bromofenac, meloxicam, ibuprofen, ketoprofen, indomethacin, piroxicam, etodolac, flurbiprofen, acetaminophen, ketorolac, or celecoxib. In one embodiment, the NSAID is celecoxib.

In one embodiment, the steroid is a corticosteroid.

In one embodiment, the corticosteroid is dexamethasone loteprednol, loteprednol etabonate, clobetasol propionate, halobetasol propionate, fluocinonide, diflorasone diacetate, desoximetasone, clocortolone pivalate, mometasone furoate, triamcinolone acetonide, betamethasone valerate, fluticasone propionate, prednicarvate, probutate, triamcinolone acetonide fluocinolone acetonide, dexamethasone loteprednolloteprednol etabonate, alclometasone dipropionate, desonide or hydrocortisone.

In one embodiment, the first and second compounds and the NSAID and/or steroid are formulated into a single dosage form.

### VI. Methods of Use and Uses

The CB2RA Compositions described herein are capable of agonizing CB₂ cannabinoid receptor activity in a subject and may provide a beneficial therapeutic effect by providing analgesic and/or anti-inflammatory properties. Accordingly, the compositions described herein (including the pharmaceutical compositions) can be used to treat pain and/or inflammation in a subject in need thereof.

In certain embodiments, the pain and/or inflammation may be chronic or acute pain and/or inflammation. In certain embodiments, the pain and/or inflammation is associated with autoimmune disorders including diabetes, multiple sclerosis, psoriasis, systemic lupus erythematosus, inflammatory bowel diseases, Addison's disease, Graves' disease, Hashimoto's thyroiditis, myasthenia gravis, autoimmune vasculitis, pernicious anaemia, celiac disease and the like, or with radiation (as occurs with treatment of certain cancers), or with infection, or with exposure to irritants, or from heart disease, or from asthma, or from Alzheimer's disease or other neurological disorders. The disclosed invention may be used to treat these disorders.

The disclosed composition may also be used to treat inflammatory diseases of the joints and connective tissue such as vascular diseases of the connective tissue, sprains and fractures, and musculoskeletal diseases with inflammatory symptoms such as acute rheumatic fever, polymyalgia rheumatica, reactive arthritis, rheumatoid arthritis, spondylarthritis, and also osteoarthritis, and inflammation of the connective tissue of other origins, and collagenoses of all origins such as systemic lupus erythematodes, scleroderma, polymyositis, dermatomyositis, Sjogren syndrome, Still's disease or Felty syndrome; as well as vascular diseases such as panarteriitis nodosa, polyarthritis nodosa, periarteriitis nodosa, arteriitis temporalis, Wegner's granulomatosis, giant cell arteriitis, arteriosclerosis and erythema nodosum.

In certain embodiments, the pain results from trauma (whether for example from personal injury or from surgical procedures), from cancer or other underlying acute or chronic medical conditions, from burns, from scalding, or from exposure to chemicals or from headache or from migraine.

The composition described herein may be suitable for treating acute pain such as for example toothache, peri- and post-operative pain, traumatic pain, muscle pain, the pain caused by burns, sunburn, trigeminal neuralgia, pain caused by colic, as well as spasms of the gastro-intestinal tract or uterus; sprains, visceral pain such as for example chronic pelvic pain, gynaecological pain, pain before and during menstruation, pain caused by pancreatitis, peptic ulcers, interstitial cystitis, renal colic, cholecystitis, prostatitis, angina pectoris, pain caused by irritable bowel, non-ulcerative dyspepsia and gastritis, prostatitis, non-cardiac thoracic pain and pain caused by myocardial ischaemia and cardiac infarct.

The methods described herein may further comprise administering to the subject a therapeutically effective amount of a NSAID and/or a steroid. In certain embodiments, the NSAID may be bromofenac aspirin, naproxen, bromfenac, diclofenac, meloxicam, ibuprofen, ketoprofen, tolmetin, indomethacin, sulindac, piroxicam, mefenamic acid, etodolac, nepafenac, flurbiprofen, acetaminophen, bromofenac, ketorolac, celecoxib, etoricoxib, lumiroacoxib, rofecoxib, valdecoxib, parecoxib, acemethacin, dexibuprofen, nimesulide, nabumetone, tiaprofenic acid, lornoxicam, tenoxicam, aceclofenac, proglumethacin, dexketoprofen or oxaprozin. In one embodiment, the NSAID is celecoxib. In other embodiments, the steroid may be a corticosteroid for example, a corticosteroid, including a glucocorticosteroid. The corticosteroid or glucocorticosteroid may be clobetasol propionate, halobetasol propionate, fluocinonide, diflorasone diacetate, desoximetasone, clocortolone pivalate, mometasone furoate, triamcinolone acetonide, betamethasone valerate, fluticasone propionate, prednicarvate, hydrocortisone probutate, triamcinolone acetonide fluocinolone acetonide, dexamethasone loteprednolloteprednol etabonate, alclometasone dipropionate, or desonide. The NSAID and/or steroid may be administered to the subject before, simultaneous with, or after administration of the first and second compounds. In certain embodiments, the administration of a composition described herein synergistically reduces inflammation and/or pain in the subject when administered in combination with the NSAID and/or steroid.

In certain embodiments, the inflammation or pain may be ocular inflammation or ocular pain, for example, postoperative inflammation or postoperative pain or pain associated with trauma to the eye, such as occurs with corneal abrasion, or from glaucoma, or from hordeolum (or other infection), or from keratoconus, or orbital cellulitis, or from cross linking the cornea, or from burns or chemical damage to the eye, or from ulcers, or from dry-eye syndrome, or from poor tear formation or from conjunctivitis, or from optic neuritis or scleritis or uveitis or keratitis or from back of the eye conditions affecting the retina.

Corneal inflammation can lead to corneal neuropathic pain (hyperalgesia). Corneal neuropathic pain can result from an initial trauma and inflammatory response, or as a result of persistent chronic inflammation/irritation (for example, a dry eye condition). Ocular neuropathic pain conditions are often associated with corneal injury and inflammation, where inflammation is a significant contributor to neuropathic pain syndromes.

Corneal neuropathic pain typically presents with allodynia (abnormal response to normal stimuli) and hyperalgesia (exaggerated response to mild noxious stimuli). Corneal pain conditions are very common as the cornea is highly innervated with sensory nerves. Accordingly, in an embodiment, the compositions described herein can be useful in treating ocular inflammation and neuropathic pain caused by a non-infectious condition. In certain embodiments, the ocular neuropathic pain is corneal neuropathic pain. In certain embodiments, the ocular neuropathic pain arises from dry eye, trauma (for example, refractive surgery or injury), a corneal abrasion, a corneal burn, a corneal transplant, an autoimmune disease or an allergen. It will be appreciated by a person skilled in the art that such conditions typically present with both neuropathic pain and inflammation and that treatment with methods of the present application can reduce the ocular inflammation and hence the ocular neuropathic pain.

Corneal pain also occurs after surgery to the eye where the corneal epithelium is removed wholly or partially as occurs with photorefractive keratectomy. In certain embodiments, the ocular pain is caused by corneal epithelium removal followed by cross-linking of the corneal stroma to correct keratoconus including progressive keratoconus.

In certain embodiments, the eye disease causes intraocular inflammation. Optionally, the eye disease is uveitis, uveoretinitis or proliferative vitreoretinopathy. In certain embodiments, the eye disease causes extraocular inflammation. Optionally, the eye disease is corneal inflammation or neuropathology, episcleritis or scleritis. In certain embodiments, the eye disease causes pain and loss of vision, and the agent reduces the pain and/or reduces the loss of vision.

The human eye comprises a number of semi-isolated microenvironments, which, if injured or diseased, can require a variety of treatments. Pathologies of the eye can generally be divided into those occurring in (i) the anterior segment, including for example, the cornea, iris, ciliary body, trabecular meshwork and lens and the aqueous humor that contacts them, and (ii) the posterior segment that includes, for example, the vitreous humor, the retina and its various components, the choroid and the optic nerve.

Pathologies affecting the anterior segment include refractive changes associated with aging or congenital defects that may require surgical intervention (for example myopia, hyperopia, presbyopia and cataract, keratoconus and the endothelial dystrophies) as well as those that occur for example via infection or other cause of inflammation such as dry-eye, uveitis, blepharitis, corneal ulcers, and conjunctivitis. Trauma, occurring, for example, via chemical burns or foreign objects, is often an anterior segment condition. In many circumstances, the failure to receive adequate and appropriate medical intervention can result in vision impairment or vision loss.

Pathologies affecting the posterior segment at the back of the eye including those affecting the retina and the optic nerve, if not adequately and appropriately treated can also result in vision impairment or vision loss. These include wet and dry age-related macular degeneration (AMD), diabetic macular edema (DME), retinitis, retinal detachment and ocular ischaemic syndrome. Glaucoma, a sight-threatening condition where increased pressure within the eye (intraocular pressure or IOP) can result in damage the optic nerve at the back of the eye, may also be considered a posterior segment disease, though its cause is likely to be poor fluid drainage via the trabecular meshwork of the anterior segment.

Inflammation and pain are common manifestation of both anterior and posterior segment diseases, whether the cause of, or an outcome of the condition. For example, corneal surgeries may involve injury or removal to the corneal epithelium such as those undertaken to correct refractive errors including PRK (photorefractive keratectomy) and keratoconus cross-linking surgery, which can be extremely painful as the cornea is one of the most densely innervated surface epithelium of the body with a sensitivity up to 600 times that of normal skin (Yang, A. Y. et al. (2018) YALE J. BIOL. MED. 91(1): 13-21). The inflammation that occurs post-surgery may also progress and intensify the pain that, in some patients, leads to more chronic inflammatory conditions such as dry-eye disease (Shtein R. M. (2011) EXPERT REV. OPHTHALMOL. 6(5): 575-582), a multifactorial condition of the ocular surface that often results in severe discomfort and visual disturbance, with the potential to cause permanent damage to the corneal surface (Javadi M A, Feizi S. (2011) J. OPHTHALMIC VIS. RES. 6(3): 192-198).

Conversely glaucoma, and the optic nerve damage it causes, is believed to arise in certain circumstances as a consequence of inflammation and blockage of the trabecular network (Ozan-Yuksel Tektas et al. (2009) EXPER. EYE RES. 88(4): 769-775). Inflammation is likewise considered a major factor in the generation of both forms of AMD being of particular importance in dry AMD the most prevalent of the conditions (Kauppinen, A. et al. (2016) CELL. MOL. LIFE SCI. 73(9): 1765-868). Current treatments for ocular pain and inflammation (as opposed to treatments to prevent causes of inflammation such as anti-microbial products) generally fall into three categories including (i) oral opioid medicines, non-steroidal anti-inflammatory drugs (NSAID), and (iii) corticosteroids. Opioid oral medicines are analgesics that while commonly employed for the most severe ophthalmic pain, as occurs after cross-linking surgery or PRK for example, are becoming less frequently prescribed because of concerns relating to misuse and abuse and an inability to effectively control pain local to the eye, post oral administration. Topical NSAIDs such as ketorolac, bromfenac and nepafenac are commonly used topical analgesics for treating ophthalmic pain and inflammation though their potency is less than that achievable by opioids. NSAIDs are also associated with a number of class related side-effects which may prevent both their short-term use, in, for example, ophthalmic surgery, and their long-term use in patients with glaucoma or dry-eye, for example. Furthermore, NSAIDS are generally associated with (i) delayed wound healing which can lead to prolonged pain and inflammation and a greater chance of infection and keratitis, and (ii) increased bleeding times and, in as short a time as 14 days of repeated use, can cause elevated IOP leading to detrimental outcomes for those suffering glaucoma (Kashiwagi K et al. (2003) BR. J. OPHTHALMOL. 87297-301). Corticosteroids such as loteprednol, dexamethasone and prednisolone are used extensively to control inflammation in the eye, but not the severe acute pain that can occur after ophthalmic surgery because their mode of action at the genetic level precludes a rapid onset of action. Ophthalmic use of ocular corticosteroids has also been associated with the formation of cataracts and clinically significant elevations in IOP, and subsequent potential for glaucoma, again a likelihood that increases with the duration of treatment.

The compositions provided herein solve unmet medical needs, which include a need for new analgesic and anti-inflammatory medicines that provide early onset analgesia to a degree greater than that which can be achieved with NSAIDs or corticosteroids that are not susceptible to misuse and abuse, and which are not associated with increased bleeding times, delays to wound healing and increases in IOP.

Drug delivery to the inner chambers of the eye from the general circulation (e.g. post oral or parenteral delivery) is hindered by the blood-retinal barrier of the posterior segment and the blood aqueous barrier of anterior the segment which together prevent effective drug intraocular drug concentrations being achieved (Sharma, M. et al. (2019) METHODS IN MICROBIOL. 46: 93-114). Topical, or invasive intracameral, intravitreal or periocular injections are now the preferred routes to overcome these barriers but all are associated with various drawbacks including, for example, (i) intravitreal and intracameral injections of liquid into the eye can increase IOP and risk damage to and infection of the ocular tissues, (ii) periocular injections around the eye while at least local to their target tissues must also traverse the outer layers of the eye (sclera and choroid) before access to the inner chambers can occur and such injections can only be performed by trained physicians, and (iii) topical delivery, which although it can overcome the foregoing challenges, is also problematic due to the rapid turnover of tear fluid covering the ocular surface, blinking to remove applied liquids and the hydrophilic nature of the corneal epithelium which forms an effective barrier to lipophilic drug delivery.

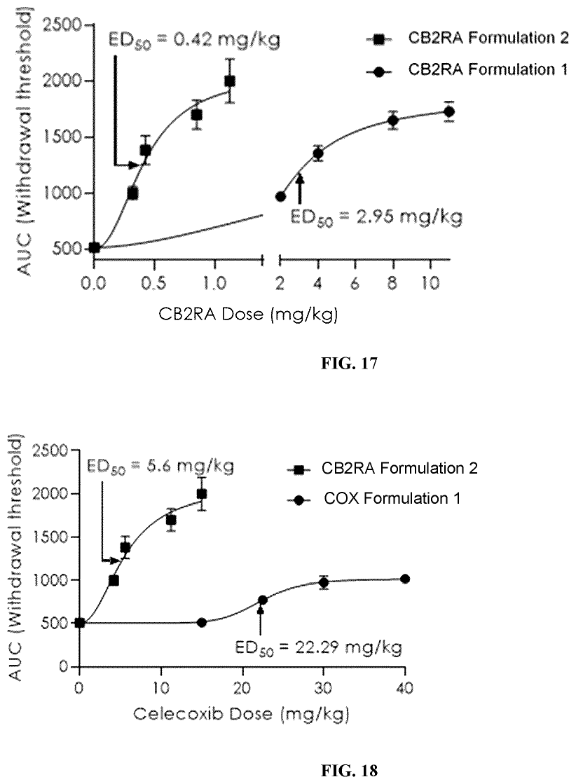

The mammalian eye is known to express CB2 receptors differentially in the cornea, the trabecular meshwork, and Schlemm's canal of the anterior segment and, in the posterior segment in the retinal Muller cells, the retinal pigment epithelium and the horizontal and amacrine cells. Although it has been discovered that the CB2R compositions described herein provide potent local analgesia in a model of surgical injury after systemic intravenous delivery (see, Example 5), it has also been discovered that analgesia can be achieved after topical delivery of a PVP-PLA CB2RA composition to the injured eye of a test subject (see, Example 25). For example, FIG. 20 shows that when delivered topically to rodent eyes cauterized with silver nitrate to mimic epithelium damage in e.g. keratoconus, PRK or LASIK surgeries, then after subsequent capsaicin challenge, test subjects receiving a topically applied amount of a pharmaceutical formulation comprising a CB2RA composition (with a ratio of E1 to E2 of 98.8 to 1.2), with a CB2RA composition concentration of 0.5%, experienced a level of pain that was lower than that experienced by animals where only vehicle (a pharmaceutical formulation lacking the CB2RA composition) was applied. Furthermore, the degree of pain reduction experienced by the test subjects was equal to that generated by a pharmaceutical composition comprising Hu-308 at a concentration of 1.5% as described in Thapa et al (2018) supra, the 1.5% Hu-308 composition concentration being the lowest dose reported to be effective.

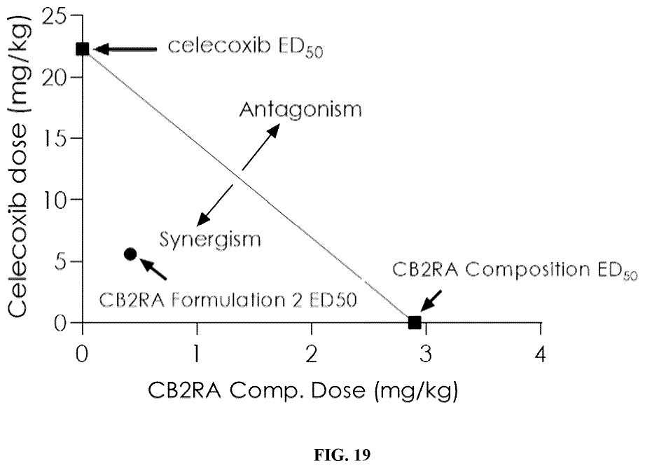

FIG. 21 shows that delivery of the CB2RA composition to the surface of the eye using the PVP-PLA formulation also resulted in a reduction in the number of leukocytes (neutrophils) that accumulated in the sub-strata of the cornea demonstrating that the PVP-PLA formulation achieved levels of CB2RA in the layers of the cornea below the surface epithelium, that were demonstrably anti-inflammatory in that they reduced the degree of leukocyte binding and accumulation. It was also discovered that, a decrease in the concentration of the CB2RA composition applied as a PVP-PLA formulation to the eye from 0.5% to 0.25% surprisingly resulted in an increased amount of analgesia for test subject.

Methods

In one aspect, there is provided a method of treating inflammation or a pain in a subject in need thereof, the method comprising administering to the subject a therapeutically effective amount of the composition as defined herein.

In one aspect, there is provided a method of treating inflammation and pain in a subject in need thereof, the method comprising administering to the subject a therapeutically effective amount of the composition as defined herein.

In one embodiment, the inflammation or the pain is chronic or acute.

In one embodiment, the pain is acute pain.

In one embodiment, the inflammation or pain is ocular inflammation or ocular pain.

In one embodiment, the inflammation or pain is postoperative inflammation or postoperative pain.

In one embodiment, the inflammation or pain are associated with corneal trauma (e.g., corneal surgery or injury).

In one embodiment, the ocular inflammation is dry AMD-associated retinal inflammation.

In one aspect, there is provided a method of treating or preventing age-related macular degeneration (AMD) in a subject in need thereof, the method comprising administering to the subject a therapeutically effective amount of the composition as defined herein.

In one embodiment, the AMD is wet AMD.

In one embodiment, the AMD is dry AMD.

In one aspect, there is provided a method of treating or preventing angiogenesis in a subject in need thereof, the method comprising administering to the subject a therapeutically effective amount of the composition as defined herein.

In one embodiment, the angiogenesis is ocular angiogenesis.

In one embodiment, the ocular angiogenesis is retinal angiogenesis.

In one embodiment, the ocular angiogenesis is choroidal angiogenesis.

In one embodiment, the retinal or choroidal angiogenesis is wet AMD-associated angiogenesis.

In one embodiment, the retinal or choroidal angiogenesis is dry AMD-associated angiogenesis.

In one aspect, there is provided a method of promoting wound healing in a subject in need thereof, the method comprising administering to the subject a therapeutically effective amount of the composition as defined herein.

In one embodiment, the wound healing is ocular wound healing.

In one embodiment, the ocular wound healing is corneal wound healing.

In one embodiment, the ocular wound healing is post-operative ocular wound healing.

In one aspect, there is provided a method of preventing or treating dry eye syndrome in a subject in need thereof, the method comprising administering to the subject a therapeutically effective amount of the composition as defined herein.

In one aspect, there is provided a method of pan-ocular delivery of:
(a) a first compound of Formula I:

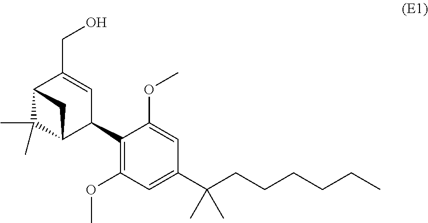 [SMILES: CCCCCCC(C)(C)c1cc(OC)c([C@@H]2C=C(CO)[C@@H]3C[C@H]2C3(C)C)c(OC)c1]

or a pharmaceutically acceptable salt thereof; and
(b) a second compound of Formula II:

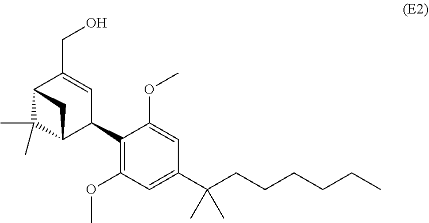 [SMILES: CCCCCCC(C)(C)c1cc(OC)c([C@@H]2C=C(CO)[C@@H]3C[C@H]2C3(C)C)c(OC)c1]

or a pharmaceutically acceptable salt thereof;

In one aspect, there is provided a method of pan-ocular delivery of:
a first compound of Formula I:

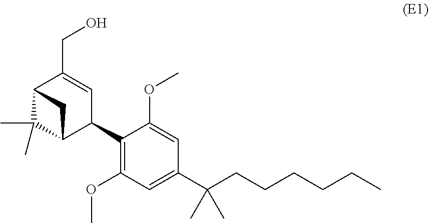 [SMILES: CCCCCCC(C)(C)c1cc(OC)c([C@@H]2C=C(CO)[C@@H]3C[C@H]2C3(C)C)c(OC)c1]

or a pharmaceutically acceptable salt thereof; and
(b) a second compound of Formula II:

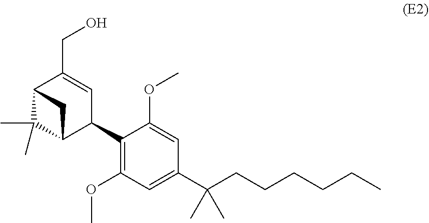 [SMILES: CCCCCCC(C)(C)c1cc(OC)c([C@@H]2C=C(CO)[C@@H]3C[C@H]2C3(C)C)c(OC)c1]

or a pharmaceutically acceptable salt thereof;

wherein the composition comprises the compound of Formula I and the compound of Formula II in a weight ratio of from 99.85:0.15 to 93.5:6.5, and a pharmaceutically acceptable excipient,
to a subject in need thereof, wherein the method comprises administering to the subject a therapeutically effective amount of the composition as defined herein that comprises E1 or pharmaceutically acceptable salt thereof, E2 or pharmaceutically acceptable salt thereof, and the PVP-PLA copolymer.

In one embodiment, the step of administering is intraperitoneal administering, topical administering, oral administering, sublingual administering, bucchal administering, intravenous administering, intramuscular administering, subcutaneous administering, intrathecal administering, otic administering, transdermal administering, intranasal administering, sublabial administering, pulmonary administering, intracranial administering, intracerebroventricular administering, intravaginal administering, rectal administering, cutaneous administering, enteral administering, periocular administering, intravitreal administering, or subconjunctival administering.

In one embodiment the composition is administered as a depot formulation, an immediate release formulation or a modified release formulation.

In one aspect, there is provided a method of increasing therapeutic efficacy of an NSAID comprising co-administering the NSAID with the composition as defined herein.

In one embodiment, the NSAID is bromofenac, nepafenac, aspirin, naproxen, diclofenac, bromofenac, meloxicam, ibuprofen, ketoprofen, indomethacin, piroxicam, etodolac, flurbiprofen, acetaminophen, ketorolac, or celecoxib. In one embodiment, the NSAID is celecoxib.

In one embodiment, the method further comprises administering to the subject a therapeutically effective amount of a nonsteroidal anti-inflammatory drug (NSAID) and/or a steroid.

In one embodiment, the NSAID is selected from nepafenac, aspirin, naproxen, diclofenac, bromofenac, meloxicam, ibuprofen, ketoprofen, indomethacin, piroxicam, etodolac, flurbiprofen, acetaminophen, bromofenac, ketorolac, or celecoxib. In one embodiment, the NSAID is celecoxib.

In one embodiment, the steroid is a corticosteroid or a glucocorticosteroid.

In one embodiment, the corticosteroid is dexamethasone, presnisalone loteprednol, fluocinolone, fluoromethalone, difluprednate, triamcinolone or rimexolone.

In one embodiment, the glucocorticosteroid is clobetasol propionate, halobetasol propionate, fluocinonide, diflorasone diacetate, desoximetasone, clocortolone pivalate, mometasone furoate, triamcinolone acetonide, betamethasone valerate, fluticasone propionate, prednicarvate, probutate, triamcinolone acetonide fluocinolone acetonide, loteprednolloteprednol etabonate, alclometasone dipropionate, desonide or hydrocortisone.

In one embodiment, the NSAID and/or steroid is administered to the subject before, simultaneous with, or after administration of the first and second compounds.

In one embodiment, the administration of the composition as defined herein synergistically reduces inflammation and/or pain in the subject when administered in combination with the NSAID and/or steroid. In one embodiment, the NSAID is celecoxib.

Uses/Compositions for Use (Supra)

In one aspect, there is provided a use of the composition as defined herein for treatment of inflammation or pain in a subject in need thereof.

In one aspect, there is provided a use of the composition as defined herein for preparation of a medicament for treatment of inflammation or pain in a subject in need thereof.

In one aspect, there is provided the composition as defined herein for use in for treatment of inflammation or pain in a subject in need thereof.

In one embodiment, the inflammation or the pain is chronic pain or acute pain.

In one embodiment, the pain is acute pain.

In one embodiment, the inflammation or pain is ocular inflammation or ocular pain.

In one embodiment, the inflammation or pain is postoperative inflammation or postoperative pain.

In one embodiment, the inflammation or pain are associated with corneal trauma (e.g., corneal surgery or injury).

In one embodiment, the ocular inflammation is dry AMD-associated retinal inflammation.

In one aspect, there is provided a use of the composition as defined herein for treatment of age-related macular degeneration (AMD) in a subject in need thereof.

In one aspect, there is provided a use of the composition as defined herein for preparation of a medicament for treatment of age-related macular degeneration (AMD) in a subject in need thereof.

In one aspect, there is provided the composition as defined herein for use in treatment of age-related macular degeneration (AMD) in a subject in need thereof.

In one embodiment, the AMD is wet AMD.

In one embodiment, the AMD is dry AMD.

In one aspect, there is provided a use of the composition as defined herein for treatment or prevention of angiogenesis in a subject in need thereof.

In one aspect, there is provided a use of the composition as defined herein for preparation of a medicament for treatment or prevention of angiogenesis in a subject in need thereof.

In one aspect, there is provided the composition as defined herein for use in treatment or prevention of angiogenesis in a subject in need thereof.

In one embodiment, the angiogenesis is ocular angiogenesis.

In one embodiment, the ocular angiogenesis is retinal angiogenesis.

In one embodiment, the retinal angiogenesis is wet AMD-associated angiogenesis.

In one aspect, there is provided a use of the composition as defined herein for promotion of wound healing in a subject in need thereof.

In one aspect, there is provided a use of the composition as defined herein for preparation of a medicament for promotion of wound healing in a subject in need thereof.

In one aspect, there is provided the composition as defined herein for use in promotion of wound healing in a subject in need thereof.

In one embodiment, the wound healing is ocular wound healing.

In one embodiment, the ocular wound healing is corneal wound healing.

In one embodiment, the ocular wound healing is post-operative ocular wound healing.

In one aspect, there is provided a use of the composition as defined herein for prevention or treatment of dry eye syndrome in a subject in need thereof.

In one aspect, there is provided a use of the composition as defined herein for preparation of a medicament for prevention or treatment of dry eye syndrome in a subject in need thereof.

In one aspect, there is provided a use of the composition as defined herein for use in prevention or treatment of dry eye syndrome in a subject in need thereof.

In one aspect, there is provided the composition as defined herein for use in prevention or treatment of dry eye syndrome in a subject in need thereof.

In one aspect, there is provided use of the composition as defined herein that comprises E1 or pharmaceutically acceptable salt thereof, E2 or pharmaceutically acceptable salt thereof, and the PVP-PLA copolymer for pan-ocular delivery of:
(a) the first compound of Formula I:

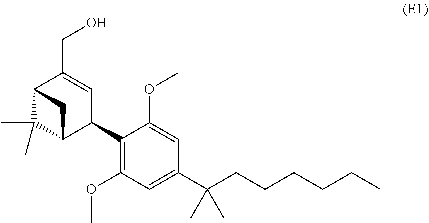 [SMILES: CCCCCCC(C)(C)c1cc(OC)c([C@@H]2C=C(CO)[C@@H]3C[C@H]2C3(C)C)c(OC)c1]

or the pharmaceutically acceptable salt thereof; and
(b) the second compound of Formula II:

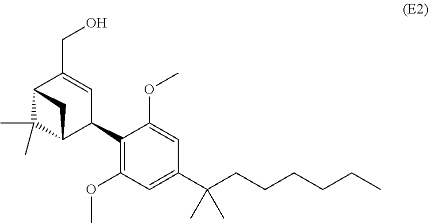 [SMILES: CCCCCCC(C)(C)c1cc(OC)c([C@@H]2C=C(CO)[C@@H]3C[C@H]2C3(C)C)c(OC)c1]

or the pharmaceutically acceptable salt thereof;
wherein the composition comprises the compound of Formula I and the compound of Formula II in a weight ratio of from 99.85:0.15 to 93.5:6.5, and a pharmaceutically acceptable excipient,
to a subject in need thereof.

In one aspect, there is provided use of the composition as defined herein that comprises E1 or pharmaceutically acceptable salt thereof, E2 or pharmaceutically acceptable salt thereof, and the PVP-PLA copolymer for preparation of a medicament for pan-ocular delivery of:
(a) the first compound of Formula I:

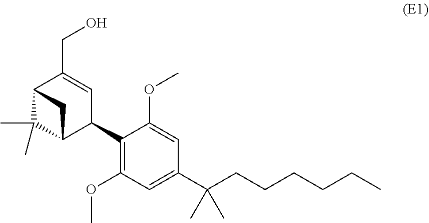 [SMILES: CCCCCCC(C)(C)c1cc(OC)c([C@@H]2C=C(CO)[C@@H]3C[C@H]2C3(C)C)c(OC)c1]

or the pharmaceutically acceptable salt thereof; and
(b) the second compound of Formula II:

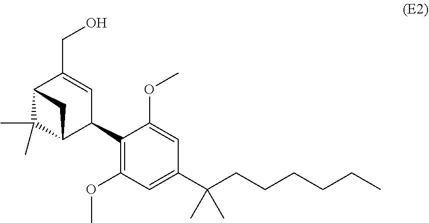 [SMILES: CCCCCCC(C)(C)c1cc(OC)c([C@@H]2C=C(CO)[C@@H]3C[C@H]2C3(C)C)c(OC)c1]

or the pharmaceutically acceptable salt thereof;
wherein the composition comprises the compound of Formula I and the compound of Formula II in a weight ratio of from 99.85:0.15 to 93.5:6.5, and a pharmaceutically acceptable excipient,
to a subject in need thereof.

In one aspect, there is provided the composition as defined herein that comprises E1 or pharmaceutically acceptable salt thereof, E2 or pharmaceutically acceptable salt thereof, and the PVP-PLA copolymer for use in pan-ocular delivery of:
(a) the first compound of Formula I:

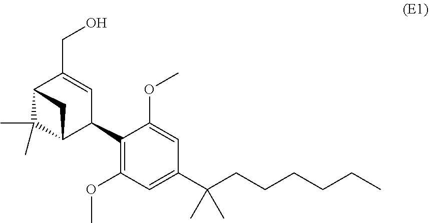 [SMILES: CCCCCCC(C)(C)c1cc(OC)c([C@@H]2C=C(CO)[C@@H]3C[C@H]2C3(C)C)c(OC)c1]

or the pharmaceutically acceptable salt thereof; and
(b) the second compound of Formula II:

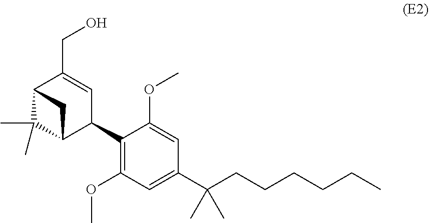 [SMILES: CCCCCCC(C)(C)c1cc(OC)c([C@@H]2C=C(CO)[C@@H]3C[C@H]2C3(C)C)c(OC)c1]

or the pharmaceutically acceptable salt thereof;
wherein the composition comprises the compound of Formula I and the compound of Formula II in a weight ratio of from 99.85:0.15 to 93.5:6.5, and a pharmaceutically acceptable excipient,
to a subject in need thereof.

In one embodiment, composition is formulated for intraperitoneal use, topical use, oral use, sublingual use, bucchal use, intravenous use, intramuscular use, subcutaneous use, intrathecal use, otic use, transdermal use, intranasal use, sublabial use, pulmonary use, intracranial use, intracerebroventricular use, intravaginal use, rectal use, cutaneous use, enteral use, periocular use, intravitreal use, or subconjunctival use.

In one aspect, there is provided a use of the composition as defined in herein for increasing therapeutic efficacy of an NSAID.

In one embodiment, the NSAID is bromofenac, nepafenac, aspirin, naproxen, diclofenac, bromofenac, meloxicam, ibuprofen, ketoprofen, indomethacin, piroxicam, etodolac, flurbiprofen, acetaminophen, ketorolac, or celecoxib. In one embodiment, the NSAID is celecoxib.

In one embodiment, the use further comprises use of a therapeutically effective amount of a nonsteroidal anti-inflammatory drug (NSAID) and/or a steroid.

In one embodiment, the NSAID is selected from nepafenac, aspirin, naproxen, diclofenac, bromofenac, meloxicam, ibuprofen, ketoprofen, indomethacin, piroxicam, etodolac, flurbiprofen, acetaminophen, bromofenac, ketorolac, or celecoxib. In one embodiment, the NSAID is celecoxib.

In one embodiment, the steroid is a corticosteroid or a glucocorticosteroid.

In one embodiment, the corticosteroid is dexamethasone, presnisalone loteprednol, fluocinolone, fluoromethalone, difluprednate, triamcinolone or rimexolone.

In one embodiment, the glucocorticosteroid is clobetasol propionate, halobetasol propionate, fluocinonide, diflorasone diacetate, desoximetasone, clocortolone pivalate, mometasone furoate, triamcinolone acetonide, betamethasone valerate, fluticasone propionate, prednicarvate, probutate, triamcinolone acetonide fluocinolone acetonide, loteprednolloteprednol etabonate, alclometasone dipropionate, desonide or hydrocortisone.

In one embodiment, the NSAID and/or steroid is for use before, simultaneous with, or after administration of the first and second compounds.

In one embodiment, the use is for synergistic reduction of inflammation and/or pain in the subject when the use is in combination with the NSAID and/or steroid. In one embodiment, the NSAID is celecoxib.

### VII. Kits for Use in Medical Applications and Commercial Packages

Another aspect, the invention provides a kit for alleviating pain and/or inflammation in a subject. In certain embodiments, the invention provides a kit comprising a CB2RA Composition for agonizing CB₂ cannabinoid receptor activity in a subject and instructions for alleviating the pain and/or inflammation in the subject. In certain embodiments, the kit further comprises one of more of multiple dosage units containing the CB2RA Composition.

In one embodiment, the kit comprises the composition as herein described or the pharmaceutical composition as herein described and instructions for a use as herein described. In some embodiments, the composition or the pharmaceutical composition present in the kit is essentially free of oxygen. For example, the composition may be stored under and inert gas. The composition or the pharmaceutical composition may be free of oxygen.

In one aspect, there is provided a commercial package comprising the composition as herein described or the pharmaceutical composition as herein described together with suitable packaging, wherein the composition is essentially free of oxygen. For example, the composition or the pharmaceutical composition may be stored under and inert gas. The composition or the pharmaceutical composition may be free of oxygen.

The description above describes multiple aspects and embodiments of the invention, including compositions, medical kits, and methods for making and using such compositions to treat alleviate pain and/or inflammation in a subject.

### EXAMPLES

The invention now being generally described, will be more readily understood by reference to the following examples, which are included merely for purposes of illustration of certain aspects and embodiments of the present invention, and are not intended to limit the invention.

### Example 1. Quantification of E1 and E2 in Lots of HU-308

This Example describes the characterization of certain commercially available lots of HU-308 with respect to optical purity and amounts of the compound of Formula I (E1) and the compound of Formula II (E2) measured in these lots.

The amounts of the enantiomers E1 and E2 in six lots of HU-308 were determined by rapid ultra-performance convergence chromatography using UPC2 system (Waters) with a Trefoil AMY1 column (2.5 μm, 3.0×150 mm). Gradient elution was achieved using a binary system of supercritical CO₂ and 20 mM ammonium acetate in methanol, with a back pressure of 2,100 psi, a flow rate of 1.2 mL/min, and DAD detection (Waters) 230 to 300 nm were applied. Under these experimental conditions, retention times of E1 and E2 enantiomers were 3.53 and 3.69 min, respectively.

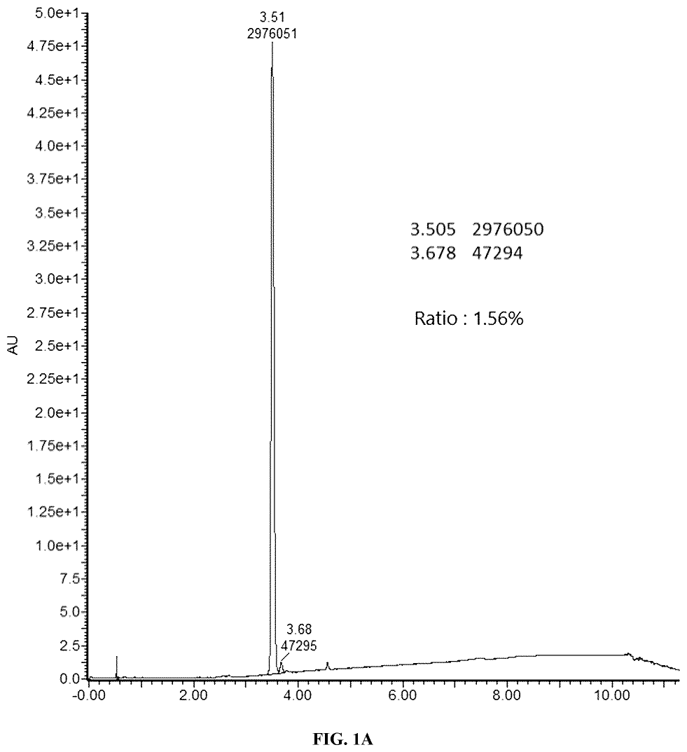

FIGS. 1A to 1F shows exemplary UPC2 chromatograms obtained for different lots tested. The chiral composition of various Lots with respect to E1 and E2 amounts are shown in Table 3.

TABLE 3
Enantiomer (E1 and E2) Amounts in Lots of Hu-
308 as Measured by UPC2/DAD Chromatography.

| | Enantiomer Concentration (weight | | |
|---|---|---|---|
| | percentage) | | Enantiomeric excess |
| Lot No. | Amount of E1 | Amount of E2 | of E1 (EE %) |
| 001 | 99.4% | 0.6% | 98.8% |
| 003 | 98.8% | 1.2% | 97.6% |
| 004 | 98.4% | 1.6% | 96.8% |
| 005 | 93.6% | 6.4% | 87.2% |
| 007 | 99.3% | 0.7% | 98.6% |
| 009 | 82.4% | 17.6% | 64.8% |

### Example 2. Purity Analysis of Lots of HU-308 by Reverse Phase HPLC

This Example shows the purity of the six lots of HU-308 described in Example 1 as determined by reverse phase HPLC (RPC).

The analysis was conducted on the batches of HU-308 using an Agilent 1100 HPLC system equipped with a quaternary pump, column heating compartment and diode array detector. A Zorbax-Eclipse XDB-C8 5 μm, 15×4.6 mm column (Agilent Technologies) was used for the analysis with a mobile phase containing a gradient mixture of water and acetonitrile. The gradient program (time/% acetonitrile) was set as 0/50, 20/100, 30/100, 40/50, and the flow rate was 1.0 mL/min. The column temperature was maintained at 30° C. and the chromatography was monitored at 210 nm (bandwidth 4 nm). Injection volume was 10 μL. Samples were prepared at 0.08 mg/mL in acetonitrile.

Under these conditions, the elution time of both entantiomers E1 and E2 was 19.9 min. Other peaks observed in the resulting RPC chromatograms and the associated purity of entantiomers E1 and E2 for the various lots, as determined by RPC, are listed in Table 4. Compounds with peaks eluting at 18.6, 19.2, 20.9, 21.4 and 21.8 minutes were identified in certain of the lots. FIG. 2 shows RPC traces recorded for two exemplary lots of HU-308.

TABLE 4
Retention Times and Associated Purities of Components

| | | | | | | | % purity of |
|---|---|---|---|---|---|---|---|
| | | | | | | | composition |
| | Retention time (min.)* | | | | | | containing |
| Lot No. | 18.6 | 19.2 | 19.9 | 20.9 | 21.4 | 21.8 | both E1 and E2 |
| 001 | + | | + | + | + | | 98.9% |
| 003 | + | | + | + | + | + | 98.7% |
| 004 | + | | + | + | | | 99.2% |
| 005 | + | | + | + | + | | 98.8% |
| 007 | + | | + | + | + | | 91.7% |
| 009 | | + | + | | + | | 98.7% |

*The retention time of the peak containing both entantiomers E1 and E2 was 19.9 minutes (shown in bold).

### Example 3. Preparation of PVP-PLA Formulation

This Example describes the formulation of a composition comprising both E1 and E2 enantiomers in PVP-PLA micelles, referred to as a CB2RA Formulation 1.

Formulations were prepared as follows. PVP-PLA block copolymer (Altus Formulation Inc.; block ratio 32:35 molecular weight 6,300) (2.45 g) was dissolved in ethanol (12.25 mL) with magnetic stirring for 10 minutes. Each lot of material described in Example 1 containing enantiomers E1 and E2 (50 mg) were separately dissolved in ethanol (10 mL) in a glass vial with magnetic stirring and mixed directly with the polymer solution at room temperature. A further amount of ethanol (10.25 mL) was then added to the mixture followed by drop wise addition of water (17.5 mL). The resulting clear solution was left under stirring for 30 minutes at room temperature.

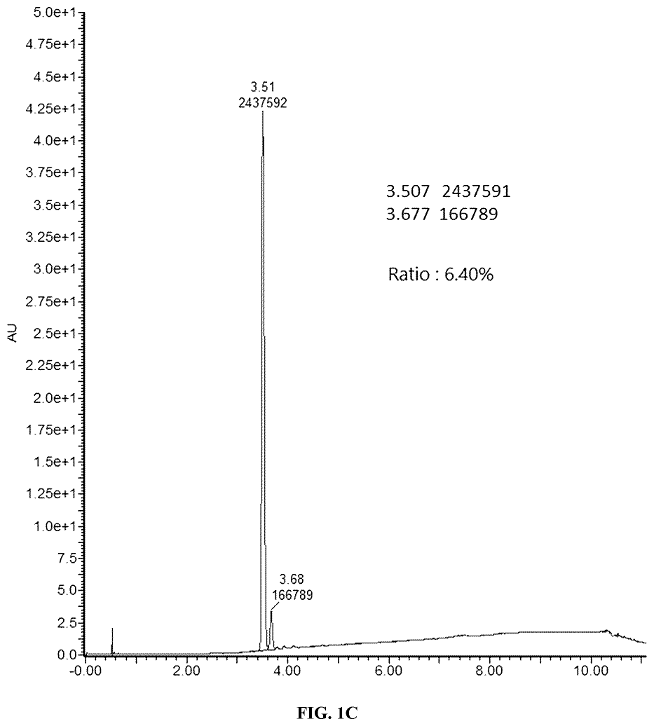

Ethanol was then removed and the PVP-PLA: E1 and E2 composition was concentrated to 10% of its initial weight using a Rocket Synergy evaporator (ThermoFisher Scientific) set in HPLC fraction mode for 150 minutes. 10×PBS buffer (5 mL) was then added to the concentrated solution followed by the addition of 2.2 mL water to obtain final concentration of the composition containing E1 and E2 to 4 g/L. The bulk solution was then filtered using a 0.2 μm filter and transferred into 10 mL glass vials. The vials were then lyophilized using a VirTis Genesis 25EL lyophilizer. A schematic representation of the formulation process is shown in FIG. 3, and the composition of the resulting lyophilized cake is shown in Table 5.

TABLE 5
Quantitative Composition of CB2RA Formulation 1 (Lyophilized)

| | | Amount/Vial by |
|---|---|---|
| Ingredients | mg/vial | Weight (%) |
| Composition containing E1 & E2 | 4.0 | 1.7% |
| PVP-PLA copolymer | 196.0 | 81.8% |
| Sodium chloride | 32.0 | 13.3% |
| Sodium hydrogen phosphate | 5.8 | 2.4% |
| Potassium dihydrogen phosphate | 1.0 | 0.5% |
| Potassium chloride | 0.8 | 0.3% |
| Total | 239.6 | 100% |

The lyophilized cakes of the various forms of the CB2RA Formulation 1 using the various of lots of material possessed a fine sponge-like structure with no cracking or collapse. All vials of material could be reconstituted rapidly (in less than 2 minutes) in water for injection (1 mL), dextrose solution (1 mL) or saline (1 mL) to generate clear, particle free solutions. The average pH of the reconstituted solution was 6.6 (+/−0.2) as measured using an pH211 pH-meter (Hanna Instruments) equipped with a gel-filled epoxy-body combination electrode.

The size (Z-average) of the micelles formed during processing were determined at 25° C. by dynamic light scattering using a Malvern Nano ZS90 Zetasizer with 90 degree scattering optics equipped with 4 mW He—Ne laser operating at 633 nm while the particle size distribution (PDI) of the micelle populations formed was calculated from maximum peak height h and its standard deviation a using the equation PDI=(σ/h)².

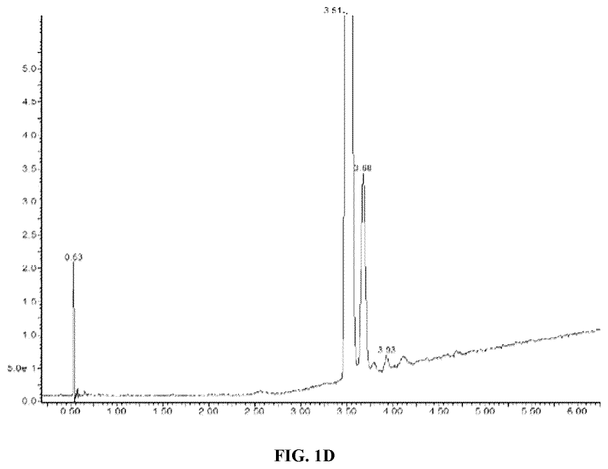

FIG. 4 provides a typical particle size distribution plot for the micelles of an exemplary CB2RA Formulation 1, which shows the distribution to be monomodal. This feature was conserved irrespective of which lot containing E1 and E2 was used. TABLE 6 summarizes the characterization data for all formulations produced and demonstrates that the physico-chemical properties of the micelle formulations do not depend on the E1 and E2 composition of the lot tested. The characteristics of the various batches of CB2RA Formulation 1 prepared also appeared not to be affected by the batch of PVP-PLA block copolymer used.

TABLE 6
Characterization Data for the Various Batches of CB2RA Formulation 1
Prepared

| CB2RA | Active | PVP- | | | | Particle size | | |
|---|---|---|---|---|---|---|---|---|
| Formulation 1/ | Agent | PLA | Amount | Amount | Enantiomeric | ZAv* | | |
| Batch No. | Lot No. | Lot No. | of E1 | of E2 | Excess (EE) | (nm) | PDi** | pH |
| A | 001 | 001 | 99.4% | 0.6% | 98.8% | 33 | 0.125 | 6.8 |
| B | 003 | 002 | 98.8% | 1.2% | 97.6% | 23 | 0.075 | 6.4 |
| C | 004 | 003 | 98.4% | 1.6% | 96.8% | 24 | 0.076 | 6.3 |
| D | 005 | 002 | 93.6% | 6.4% | 87.2% | 24 | 0.086 | 6.6 |
| E | 010 | 002 | 97.0% | 3.0% | 94.0% | 23 | 0.088 | 6.7 |
| F | 009 | 002 | 82.4% | 17.6% | 64.8% | 33 | 0.181 | 6.6 |
| | | | | | Average | 27 | 0.105 | 6.5 |

*ZAv: Micelle particle Size ; **PDi: Polydispersity Index

### Example 4. Preclinical Model for Postoperative Surgical Pain

This Example describes the production and validation of a preclinical model for post-operative surgical pain.

Pre-clinical studies to determine the ability of drug candidates to alleviate surgical pain have generally relied on models of inflammatory or neuropathic pain rather than the extreme pain experienced immediately after a surgical incision. When models of incisional pain have been employed, they typically evaluated pain responses only 24 hours after the surgical incision (Labud et al. (2005) EUROPEAN J. OF PHARMACOL., 527: 172-174) when acute pain has subsided and the inflammatory component of the acute response has been well established. Typically drug administration has also only been by the intraperitoneal route which does not mimic the clinical situation.

A pre-clinical model, representative of the extreme pain experienced immediately after surgery was developed and used to determine whether the batches of CB2RA Formulation 1 described in Example 3, were (i) capable of relieving such extreme postoperative pain and; (ii) if so, what their efficacy was relative to commercially available NSAID COX-inhibitor drugs. The model developed was based on that described by Labuda et al. (supra). However, in order to determine efficacy against acute extreme pain, pain evaluations began at once, rather than 24 hours post-surgery. All test articles were administered intravenously via the tail vein as a single bolus.

Male Sprague Dawley rats (Charles River, St-Constant, Qc, Canada), were purchased and housed in auto-ventilated cages in a climate-controlled room on a 12-hour light/dark cycle. The animals were allowed free access to food pellets and water and weighed 200-225 g at the time of the testing.

Post-operative incisional pain was induced via a 1 cm plantar incision in the hind paw. Briefly, under deep anesthesia with isoflurane the plantar aspect of the hind paw was cleaned and sterilized using a 10% povidone-iodine solution after which a 1 cm longitudinal incision was made with a number 11 blade. The incision penetrated through the skin and fascia of the plantar aspect of the foot. The plantaris muscle was also elevated and incised longitudinally, the muscle origin remaining intact. After hemostatis with gentle pressure, the skin was closed with 2 mattress sutures of 5-0 nylon and the wound site covered with an antibacterial ointment. The rats were then placed in clear Plexiglas cages with plastic grid floors (8×8 mm) and allowed to acclimate.

The pain response was evaluated using the von Frey mechanical stimulation test employing von Frey filaments applied vertically to an area adjacent to the wound. Each von Frey filament was applied once, starting at 15 mN pressure (the lowest filament strength) and continuing with filaments of increasing strength until a withdrawal response (lifting of the injured paw) was observed after application. The resistance to bending (reflective of the applied pressure) of the filament causing a withdrawal response was then recorded as the withdrawal threshold. If no withdrawal response was observed at the maximum filament strength of 522 mN then this value was recorded as the withdrawal threshold. After each filament application, animals were given a 5 minute test-free period after which the test was repeated the lowest force required for a withdrawal response from the 2 tests being recorded as the withdrawal threshold (WT).

To validate the model with respect to pain response von Frey analyses were first performed on the day before the incision to obtain a baseline response followed by a second assessment 1 hour after plantar incision.

Commercially available analgesics were employed as positive controls to validate the ability of the model to measure an analgesic response. The analgesics used were the NSAID ketorolac (Toradol®: Pfizer) and the opioid buprenorphine (available as a generic medicine).

The test article were the various batches of CB2RA Formulation 1 prepared as described in Example 3. The negative control (vehicle) was saline.

All test articles and controls were administered one hour after surgery as a single, 15 second bolus injections using a 25G needle inserted into the caudal vein, the volume to be injected being adjusted based on the individual body weight of the animal. After the first post dosing assessment at 15 minutes post injection, further assessments were then made at hourly intervals over the 8-hour test period.

### Model Validation

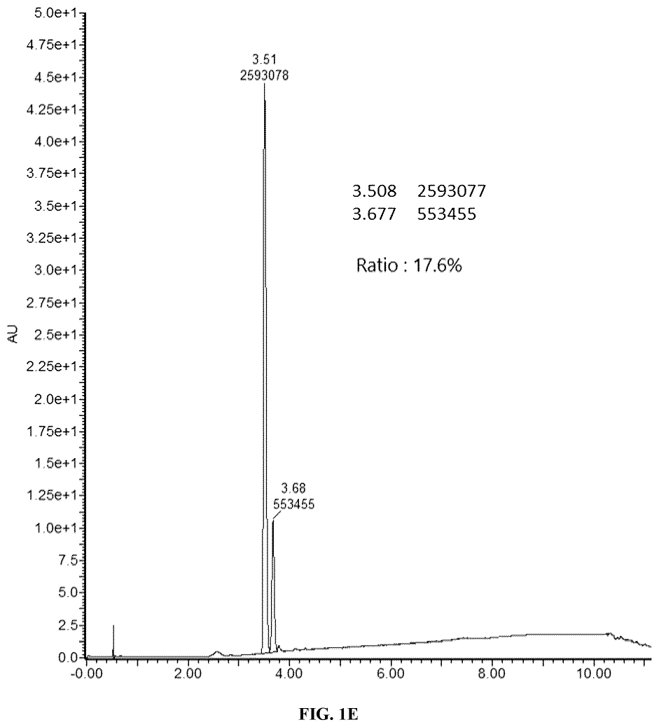

FIG. 5A shows a comparison of the withdrawal threshold measured on animals before and after plantar incision surgery. As shown, there was a significant decrease in the withdrawal threshold after surgery confirming mechanical hypersensitivity had been induced in the injured hind paw. Six animals were included in the study, which generated a response range of between about 100 mN and 400 mN which was sufficiently wide to assess the analgesic efficacy of test articles.

To validate this hypothesis, one hour after surgery separate groups of animals (n=6 per group) received a single intravenous injection of the commercially available and clinically proven NSAID ketorolac (Toradol® Pfizer). Ketorolac doses ranged from 5 mg/kg to 30 mg/kg and each group of six received only one dose. As an additional validation, a single separate group of animals also received intravenous buprenorphine (0.05 mg/kg).

The pain threshold displayed by each animal was then measured, using von Frey filaments, at 0.25, 1, 2, 3, 4, 5, 6, 7, and 8 hours after test article injection. The area under the withdrawal threshold/time curve (AUC_{0-8 hr}) was then calculated for each dose of ketorolac or buprenorphine administered, the results being presented in FIG. 5B. As shown, ketorolac displayed a clear dose response effect achieving progressive increases in response as dose increased from 5 mg/kg to 30 mg/kg, and had an ED₅₀ of 23.1 mg/kg, as shown in FIG. 5C. Buprenorphine administered at the much lower dose of 0.05 mg/kg provided as similar level of analgesia to 30 mg/kg ketorolac underlining the superior potency of opioid analgesics. Based on these findings, the model was deemed acceptable for assessment of pain and analgesia for test articles post intravenous administration.

### Example 5. Evaluation of CB2RA Formulations in the Postoperative Surgical Pain Model

This Example describes the analgesic efficacy displayed by certain of the batches of CB2RA formulation 1 described in Example 3, Table 6 in the surgical pain model described in Example 4.

In a first experiment separate groups of animals received an intravenous bolus injection of either vehicle (saline) or increasing doses of CB2RA formulation 1 Batch A (comprising weight ratio of E1 to E2 of 99.4:0.6). The doses of CB2RA Formulation 1 used for this first experiment ranged from 2-11 mg/kg with respect to the amount of the CB2RA Composition.

The paw withdrawal threshold was again measured at 0.25, 1, 2, 3, 4, 5, 6, 7, 8 hours post treatment administration animal responses being plotted as withdrawal response/time curves for each dose given as before. As shown in FIG. 6A, withdrawal thresholds greater than control were seen for all doses tested by the time of the first test point (0.25 hours) which remained above control values lasted for up to 5 hours for all doses and up to 8 hours for the 11 mg/kg dose. Analgesia was therefore immediate and lasted for up to 8 hours from a single intravenous dose.

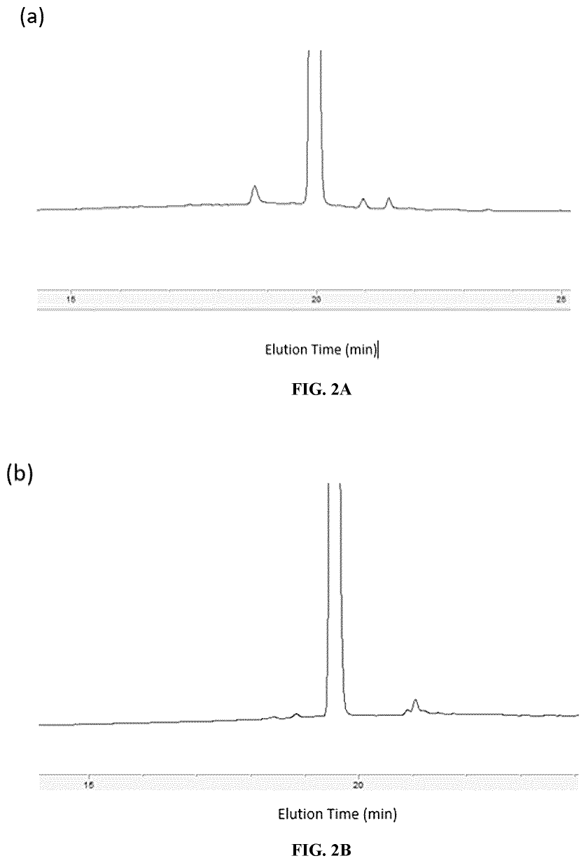

The area under the withdrawal response/time curve (AUC_{0.25-8 hr}) for each dose administered was also calculated (FIG. 6), and these values being used to determine a median effective dose (ED₅₀) value for CB2RA Formulation 1, Batch A of 2.9 mg/kg (see, FIG. 7) a value approximately 10-fold lower than the 23.1 mg/kg value calculated for ketorolac (see, FIG. 5C).

### Effect of EE Value on CB2RA Formulation Efficacy

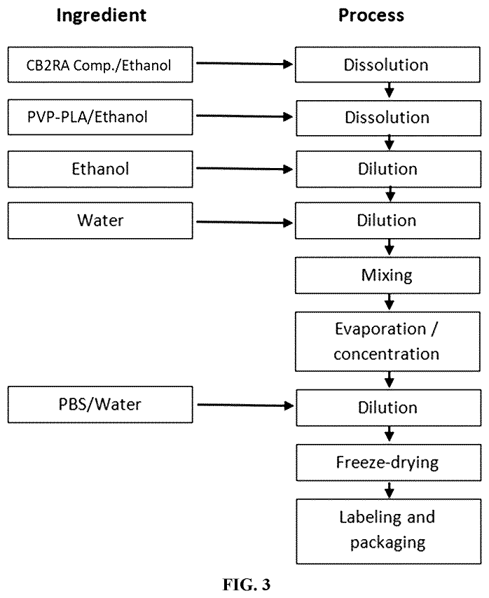

The experiment was then repeated using selected lots of CB2RA Formulation 1, namely Batch B (weight ratio of E1 to E2 of 98.8:1.2), Batch C (weight ratio of E1 to E2 of 98.4:1.6), Batch D (weight ratio of E1 to E2 of 93.6:6.4) and Batch F (weight ratio of E1 to E2 of 82.4.6:17.6). The experiments were performed at doses of 4 mg/kg and 2 mg/kg only. The 4 mg/kg dose was selected as it was central to the dose response curve of FIG. 6 and therefore represented the dose most likely to demonstrate any change in analgesic efficacy brought about by a change in E1 to E2 ratio. The 2 mg/kg dose was selected in order to determine whether a dose/response relationship was maintained between doses. The results are shown in FIGS. 8-11.

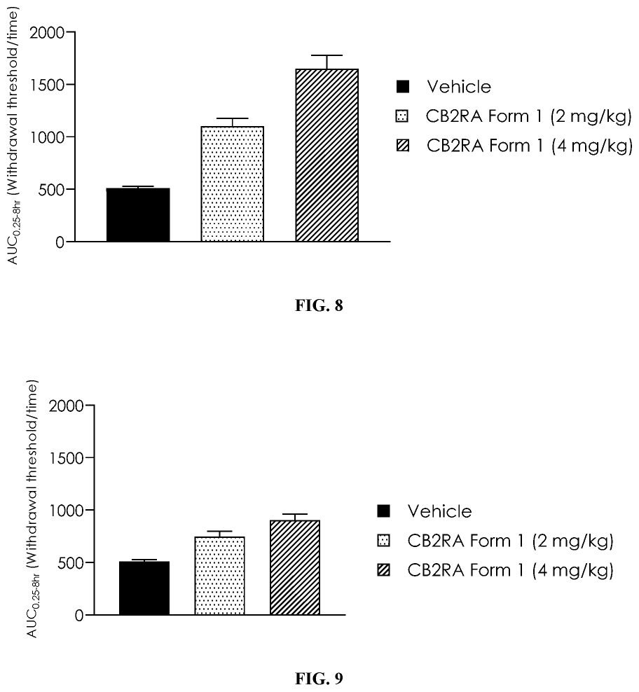

FIG. 8 shows that, when the amount of E2 in the composition was increased from 99.4:0.6 to 98.8:1.2 a pronounced analgesic effect was observed which was greater than that generated when the active ingredient contained an E1 to E2 weight ratio of 99.4:0.6 (FIG. 6B). The analgesic effect was also greater than that generated by either ketorolac or celecoxib at the same dose (see, FIGS. 5C and 14, respectively).

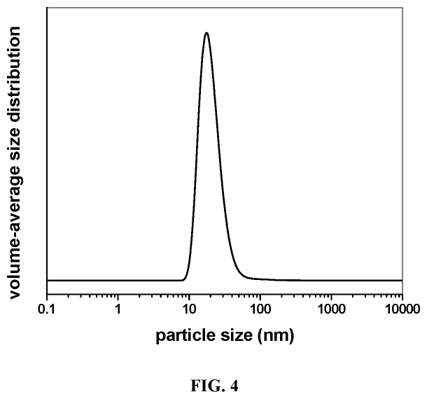

FIG. 9 shows that when the amount of E2 in the composition relative to E1 was further increased to 1.6% while analgesic effect, and a clear dose response relationship, were maintained (with again a greater degree of analgesia dose/dose than either ketorolac or celecoxib) the analgesic effect was less than that achieved by compositions containing E2 weight percentages ratios of either 0.6% or 1.2%

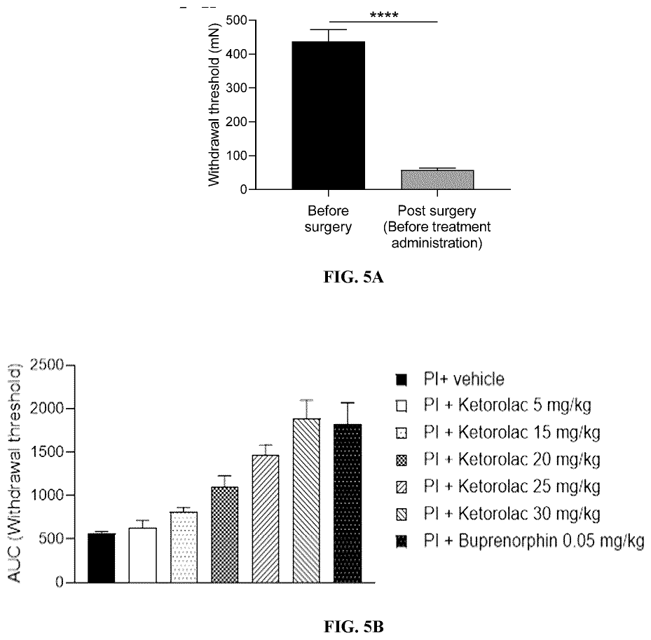

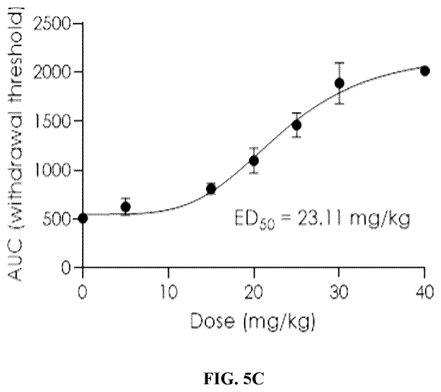

FIGS. 10 and 11 show that when compositions containing greater amounts of E2 were assessed (E2 weight percentage of 6.4 and 17.6 respectively) then analgesic effect was reduced further. For these two compositions, AUC_{0.25-8 hr} were similar for both 2 mg/kg and 4 mg/kg doses and in both cases there was no observable dose/response relationship. This lack of observable dose/response relationship suggests that compositions containing an amount of E2 greater than 6.4% would not be an effective therapeutic agent.

The change in AUC_{0.25-8 hr} for the various compositions tested, at the 4 mg/kg dosing level is summarized in FIG. 12A which displays a bell-shaped distribution of response to the effect of increasing E2 amounts in the E1 and E2 composition. FIG. 12B shows the data in FIG. 12A fitted using a Gaussian distribution.

All of the CB2RA Compositions tested generated analgesic effects greater than Control levels. Although all are analgesic; E2 amounts of below 6.4% relative to the composition display an enhanced or beneficial analgesic effect which effect is well fitted to the Gaussian distribution curve which predicts E2 amounts of between about 0.19 to about 1.78% relative to E2 will display this potency enhancing effect relative to amounts outside this range. In addition, it was discovered that the potency enhancing effect of E2 is greatest at a level of about 1%, in a composition containing E1 and E2. For example, it has been demonstrated that a CB2RA Composition where the ratio of E1 to E2 composition of 99:1 has the greatest analgesic effect in the model tested.

### Example 6. Pharmacokinetic Studies

This example describes the pharmacokinetics of CB2RA Composition as delivered by PVP-PLA formulation. This experiment was performed with the CB2RA Formulation 1, Batch A from Example 3.

The pharmacokinetics of the composition post intravenous administration were determined in a rodent model at doses shown to be analgesic in the surgery model. Briefly, male Sprague Dawley rats (Charles River, St-Constant, Qc, Canada), weighing 200-225 g at the time of the testing, were housed in auto-ventilated cages in a climate-controlled room on a 12 hour light/dark cycle. Animals were allowed free access to food pellets and water.

CB2RA Formulation 1, Batch A was administered as a single slow bolus injection into the caudal vein using 25G needle under anesthesia at doses of 2, 4 and 8 mg/kg. The volumes injected were adjusted based on the individual body weight to ensure the correct dosage was administered. Blood samples (500 μL) were collected from each animal prior to the administration of the formulations and at 0.083 hours, 0.5 hour, 1 hour, 2 hours, 4 hours, 8 hours and 12 hours following the administration by venipuncture via the right jugular vein. Five animals were used for each dose of CB2RA Formulation 1 administered. Quantitative plasma measurement of the E1/E2 composition was performed by LC-MS/MS analysis. Pharmacokinetic parameters were generated using Kinetica 5.1 software (ThermoFisher Scientific).

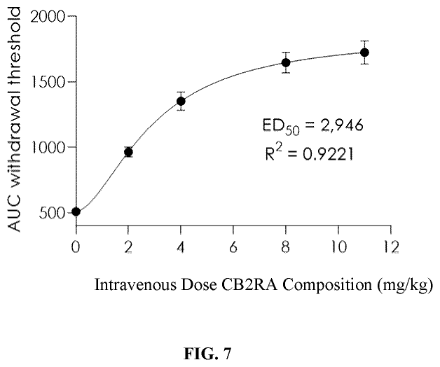

The concentrations of E1/E2 in rat plasma over time were determined by GC-MS/MS using an Agilent HP-5 ms UltraInert (30 m, 0.25 mm, 0.25 μm) analytical column employing ethyl acetate as the mobile phase. The parent product ion transition for the CB2RA Composition (m/z 318.0 233.0) was monitored on a triple quadrupol mass spectrometer (Agilent 7000C) operating in the multiple reaction monitoring (MRM) mode. The method was qualified over the concentration range of 10 to 1000 ng/mL with respect to the CB2RA Composition. The time-concentration profiles of the CB2RA Composition generated by the various PVP-PLA: formulations are summarized in Tables 7-9 and FIG. 13. Pharmacokinetic parameters as calculated by Kinetica software are summarized in Table 10.

TABLE 7
Time Concentration Profile following an IV dose of 2 mg/kg

| Time (hours) | Mean (ng/ml) | SD | CV % |
|---|---|---|---|
| 0.083 | 2142.00 | 1713.73 | 80.0% |
| 0.15 | 325.60 | 236.96 | 71.5% |
| 0.5 | 200.00 | 84.16 | 42.1% |
| 1 | 169.80 | 87.27 | 51.4% |
| 2 | 152.60 | 69.08 | 45.3% |
| 4 | 143.50 | 45.63 | 31.8% |
| 8 | 62.00 | 5.66 | 9.1% |
| 12 | BLQ | 0 | 0% |
| 24 | BLQ | 0 | 0% |

TABLE 8
Time Concentration Profile following an IV dose of 4 mg/kg

| Time (h) | Mean (ng/ml) | SD | CV % |
|---|---|---|---|
| 0.083 | 6817.50 | 4363.25 | 64.0% |
| 0.15 | 753.25 | 594.70 | 79.0% |
| 0.5 | 376.00 | 155.48 | 41.4% |
| 1 | 261.50 | 72.53 | 27.7% |
| 2 | 208.25 | 61.92 | 29.7% |
| 4 | 182.50 | 64.68 | 35.4% |
| 8 | 81.00 | 28.25 | 34.9% |
| 12 | 64.00 | 0 | 0% |
| 24 | BLQ | 0 | 0% |

TABLE 9
Time Concentration Profile following an IV dose of 8 mg/kg

| Time (h) | Mean (ng/ml) | SD | CV % |
|---|---|---|---|
| 0.083 | 13535.60 | 6924.71 | 51.2% |
| 0.15 | 2220.60 | 1004.79 | 45.2% |
| 0.5 | 901.80 | 276.07 | 30.6% |
| 1 | 468.40 | 204.04 | 43.6% |
| 2 | 363.00 | 132.50 | 36.5% |
| 4 | 282.20 | 74.32 | 26.3% |
| 8 | 129.00 | 24.28 | 18.8% |
| 12 | 66.00 | 4.24 | 6.4% |
| 24 | BLQ | 0 | 0% |

TABLE 10
Comparison of Mean PK Parameters in the Plasma

| | Dose | | |
|---|---|---|---|
| Parameter | 2 mg/kg | 4 mg/kg | 8 mg/kg |
| t_{1/2} (h) | 3.55 | 3.45 | 4.87 |
| Cₘₐₓ (ng/ml) | 2142 | 6818 | 13536 |
| C₀ (ng/ml) | 3137 | 10761 | 19658 |
| AUC₀₋ₜ (ng/ml*h) | 1392 | 3452 | 7011 |
| AUC_{0-inf} (ng/ml*h) | 1837 | 3783 | 7781 |
| AUMC (ng/ml*/kg) | 7729 | 10567 | 26388 |
| MRT (h) | 4.25 | 3.11 | 3.91 |
| Vₛₛ (mL/kg) | 4910 | 3849 | 4784 |
| V_{z} (mL/kg) | 5902 | 5644 | 8285 |
| CL (mL/h/kg) | 1217 | 1122 | 1070 |

As shown in FIG. 13, the composition delivered as CB2RA Formulation 1 exhibits a three-phase elimination profile with an initial distribution phase showing a rapid drop in concentration following by plateau and elimination phases. TABLE 10 shows that for these doses in the rat pharmacokinetics were proportional and that clearance was slow. The half-life of E1/E2 in the rat was approximately 4 hours.

### Example 7. Pharmacodynamic Evaluation of a PVP-PLA Celecoxib

This Example shows a pharmacodynamic evaluation of a PVP-PLA celecoxib formulation (COX Formulation 1).

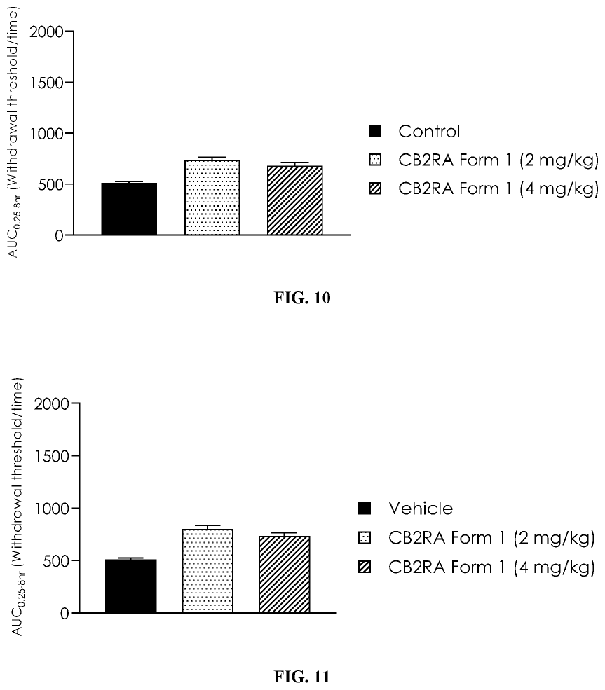

Experiments the same as those conducted in Example 6 were performed with a COX Formulation 1 prepared in the same manner as Example 3. FIG. 14 shows the dose response profile generated where doses above 22.5 mg/mg exhibited an analgesic effect greater than Control. The ED₅₀ for celecoxib in this model, as delivered by the PVP-PLA micelles was calculated as 22.3 mg/kg (see, FIG. 15) a figure similar to that generated for ketorolac (see, Example 4, and FIG. 5C) in this model.

Table 11 provides a summary of the ED₅₀ values of ketorolac celecoxib and the CB2RA Composition prepared in Example 3 (lot 1, Batch A) using PVP-PLA as a delivery agent. The CB2RA Compositions described herein containing E1/E2 are approximately 10-fold more potent than the commercially available products tested.

TABLE 11
Median effective dose (ED₅₀) of Ketorolac Formulation,
Celecoxib Formulation and CB2RA Formulation

| | ED₅₀ |
|---|---|
| Treatment | (mg/kg) |
| Ketorolac (Toradol) | 23.1 |
| Celecoxib (PVP-PLA) | 22.3 |
| CB2RA Formulation 1 (PVP-PLA) | 2.9 |

### Example 8. Combination of a CB2RA Composition and a COX Enzyme Inhibitor

This Example shows the synergy achieved when a CB2RA Composition described herein is combined with a COX enzyme inhibitor, which, in this example, was celecoxib.

The experiments were performed in order to evaluate the potential for synergy between a CB₂ receptor agonist (represented by CB2RA Composition described herein) and a COX enzyme inhibitor (represented by celecoxib) in terms of their anti-inflammatory and analgesic efficacy in the rat surgical model.

The experiments were performed as described in detail in Example 4 with the doses of CB2RA Formulation 1, Batch A and COX Formulation 1 to be mixed calculated being on the basis of their relative ED₂₅ values (see, FIGS. 7 and 15, respectively). Briefly, the ED₂₅ achieved by each formulation was determined by calculating an AUC value corresponding to 25% of the total response and by interpolating the required dose using Prism software. Doses of the reconstituted formulations were then mixed on a volume basis to achieve the required fixed dose combination, this formulation mixture was referred to as CB2RA Formulation 2. The calculated ED₂₅ values for the COX Formulation 1 and the CB2RA Formulation 1, Batch A were 11.25 mg/kg and 0.85 mg/kg, respectively, representing a ratio of the COX inhibitor to the CB2RA Composition of 13:1. Various doses of this two drug containing mixture (see, TABLE 12) were then administered intravenously to surgically treated rats as described in Example 4.

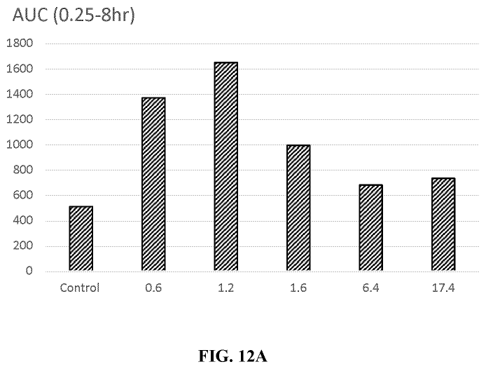

FIG. 16 shows the AUC_{(0.25-8 hr)} achieved for these combinations after intravenous administration.

TABLE 12
Dosage Forms

| | | CB2RA | | |
|---|---|---|---|---|
| Animal | Celecoxib | Composition | Ratio | No. of |
| group | dose (mg/kg) | dose (mg/kg) | Celecoxib:CB₂RA | animals |
| 1 | 4.22 | 0.32 | 13 | 6 |
| 2 | 5.62 | 0.425 | 13 | 6 |
| 3 | 11.25 | 0.85 | 13 | 6 |
| 4 | 15 | 1.125 | 13 | 6 |

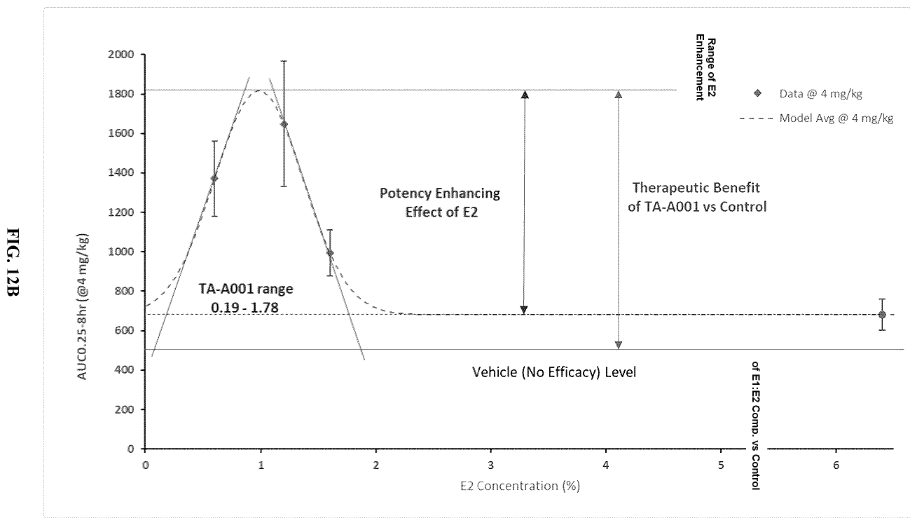

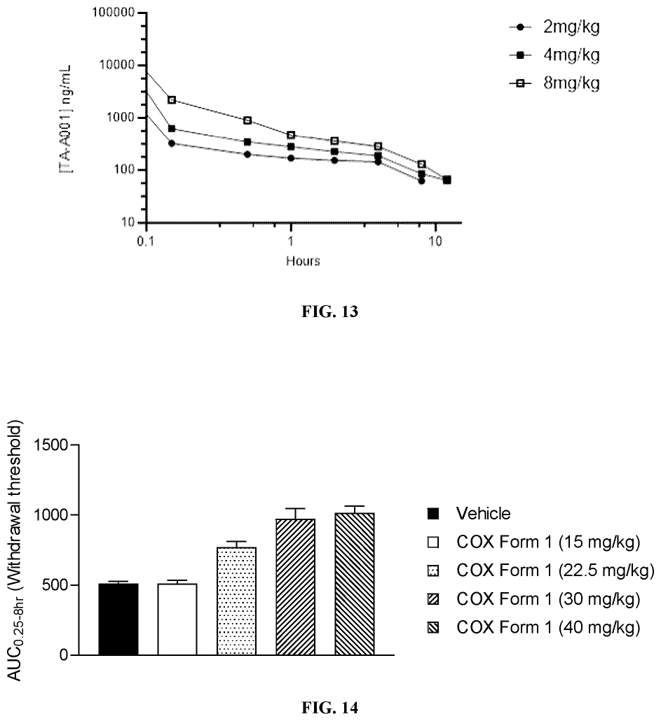

It was discovered that a significantly greater withdrawal threshold (also referred to as a significantly greater analgesia) was observed for each dose of the fixed dose combination of CB2RA Formulation 2 than was expected by simple addition of the two drug's analgesic effects. As shown in FIG. 17, the AUC versus dose curves obtained for the combinations of CB₂R agonist (FIG. 17) and COX enzyme inhibitor (FIG. 18) were shifted significantly to the left. The calculated ED₅₀ values decreased from 2.9 to 0.42 mg/kg for the CB2RA Composition alone and in combination with celecoxib, respectively, and from 22.3 to 5.6 mg/kg for celecoxib used separately and in combination with the CB2RA Composition, respectively.

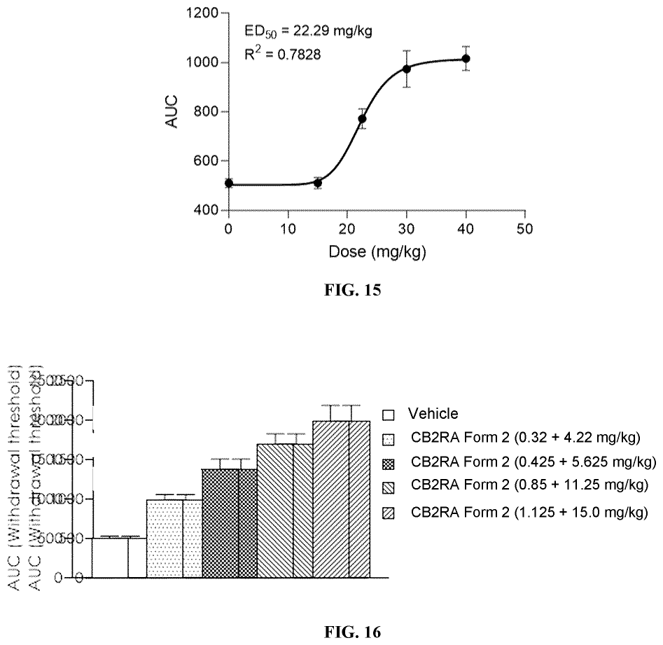

The isobologram set forth in FIG. 19 shows that the ED₅₀ value calculated for each drug separately and for the combination. The straight line connecting two points calculated for each drug separately is the theoretical additive line. If the experimental derived isobole (a point representing x, y coordinates for ED₅₀) is plotted significantly below the theoretically additive isobole, the interactive effect is identified to be synergistic. On the contrary, when the experimentally derived point for ED₅₀ is located significantly above the theoretical additive isobole, the two drugs are antagonists. FIG. 19 shows that the ED₅₀ value for the fixed dose combination fell below the additive effect line demonstrating synergy was occurring between the two drugs.

It has been discovered surprisingly that, by combining amounts of a CB2RA Composition and a COX enzyme inhibitor (e.g., celecoxib) results in a synergistic response such that, at all concentrations evaluated, the apparent potency of each drug molecule is increased thereby overcoming the ceiling effect experienced with NSAID and COX-2 drugs and allowing either effective dosing at lower overall drug concentrations, thus avoiding dose limiting adverse side-effects or higher levels of analgesia to be obtained for those in extreme pain without the need to resort to opioids.

### Example 9. Method of Synthesis

This Example describes the synthesis of a CB2RA Composition so as to control the enantiomeric ratio of E1 and E2. E1 and E2 were prepared according to the schemes shown below.

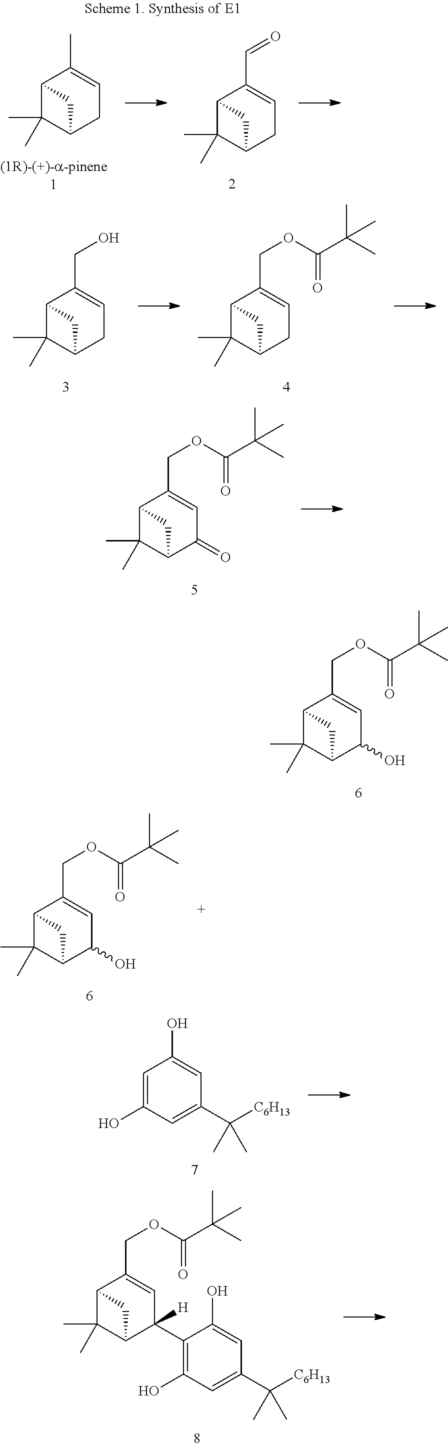

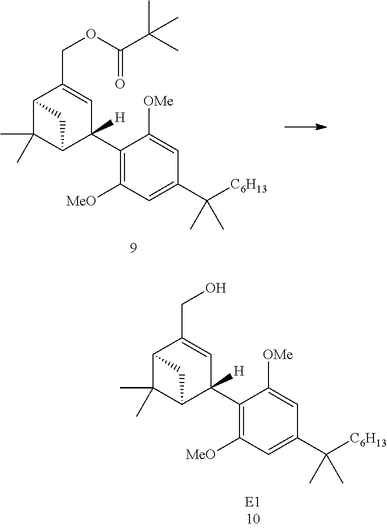

The composition the compound of Formula I (enantiomer E1) was synthesized via a series of eight steps beginning with (1R)-(+)-α-pinene as starting material. Briefly, the methyl carbon at position C2 of (1R)-(+)-α-pinene was oxidized to produce myrtenal, followed by a reduction to (+)-myrtenol. Then, the alcohol group of (+)-myrtenol was protected with a pivaloyl group. The protected (+)-myrtenol was further oxidized at the methylene carbon (C4 position) and reduced to obtain 4-hydroxy-myrtenyl pivalate. Next, the 4-hydroxy-myrtenyl pivalate was condensed with 5-(1,1-dimethylheptyl)-resorcinol affording (2-[2,6-dihydroxy-4-(2-methyloctan-2-yl)phenyl]-7,7-dimethyl-4-bicyclo[3.1.1]hept-3-enyl]pivalate). In the following step, the alcohol groups at C2 and C6 positions of the resorcinol moiety were methylated into methoxy groups. In the final step, the pivalate protecting group was removed through reduction of (2-[2,6-dimethoxy-4-(2-methyloctan-2-yl)phenyl]-7,7-dimethyl-4-bicyclo[3.1.1]hept-3-enyl]pivalate) affording the desired product (E1, the compound of Formula I).

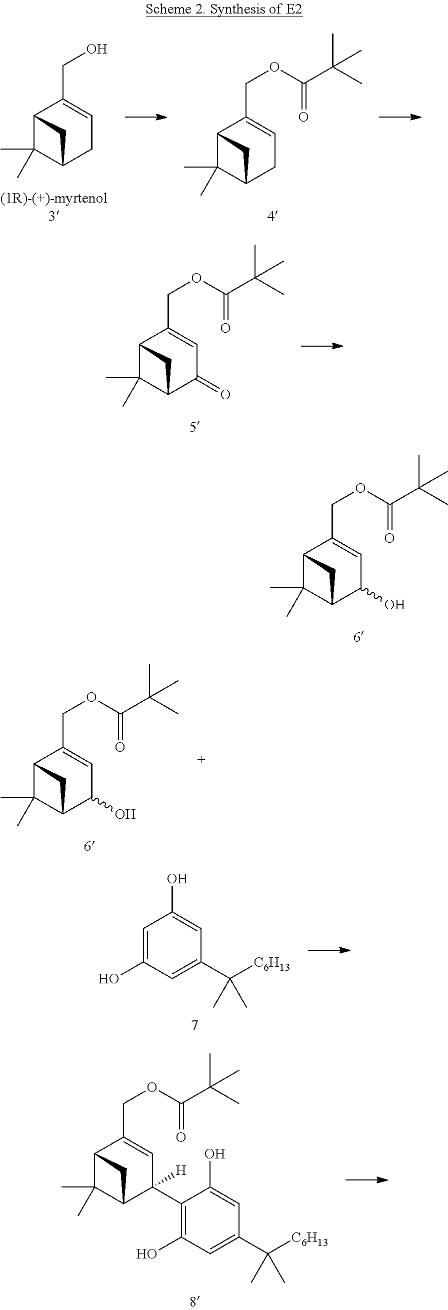

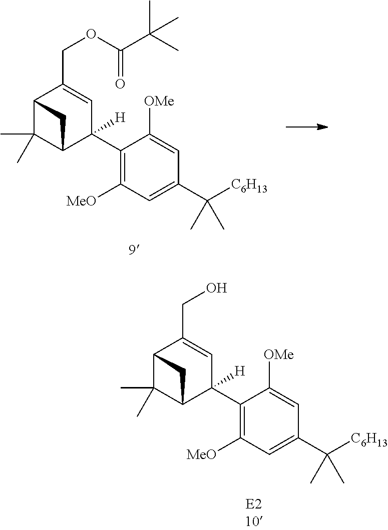

To synthesize the compound of Formula II (enantiomer E2), the alcohol group at the C2 position of (1R)-(−)-myrtenol was first protected using a pivaloyl protecting group. The protected (1R)-(−)-myrtenol was then oxidized at its C4 position to generate 4-oxo-myrtenyl pivalate which was subsequently reduced into 4-hydroxy-myrtenyl pivalate. The (1R)-(−)-4-hydroxy-myrtenyl pivalate was then condensed with 5-(1,1-dimethylheptyl)-resorcinol to give (2-[2,6-dihydroxy-4-(2-methyloctan-2-yl)phenyl]-7,7-dimethyl-4-bicyclo[3.1.1]hept-3-enyl]pivalate). In the final two steps, the alcohols at C2 and C6 positions were methylated and the pivalate protecting group removed via reduction producing the compound of Formula II (enantiomer E2).

E1 and E2 may be enantiomerically purified at the end of the synthesis via a method known to a skilled in the art (e.g., a chiral HPLC, SFC). Alternatively, the starting materials of E1 and E2 or the synthetic intermediates may be enantiomerically purified and used in the synthetic schemes. The E1/E2 ratio of a desired CB2RA Composition may be controlled by mixing known amounts of enantiomerically pure E1 and E2 and confirming the ratio by a chiral HPLC.

Two lots of an exemplary CB2RA Composition produced by these schemes 1 and 2 were produced. One lot was prepared by performing the reaction schemes separately, whereas the other lot was prepared by combining the reaction schemes so that certain steps from each scheme occurred in a single vessel. The resulting compositions were analyzed by reverse phase HPLC analysis as described in Example 2, and the results are summarized in Table 13. In Table 13, Content (%) denotes the measured amount of compound in terms of percentage composition relative to that of the entire composition and (+) denotes an identified compound with a percentage composition relative to that of the entire composition below the level of quantification of the specified analytical method.

TABLE 13
Compound Number

| | | | | CB2RA | | |
|---|---|---|---|---|---|---|
| | | | | Com- | | |
| 1 | 2 | 3 | 4 | position | 6 | 7 |

Retention Time (RT min.)

| | | | | | | |
|---|---|---|---|---|---|---|
| 17.4 | 18.1 | 18.4 | 19.2 | 19.9 | 20.2 | 23.2 |

Relative Retention Time (RRT. min)

| | | | | | | |
|---|---|---|---|---|---|---|
| 0.87 | 0.91 | 0.92 | 0.96 | 1.00 | 1.02 | 1.16 |

Content (%)

| | | | | | | | |
|---|---|---|---|---|---|---|---|
| Lot 1 | 0.32 | + | 0.65 | + | 98.18 | 0.13 | 0.08 |
| Lot 2 | 0.54 | 0.11 | 0.29 | 0.13 | 98.01 | 0.09 | + |

The CB2RA Composition (i.e, containing enantiomers E1 and E2) has a retention time of 19.9 minutes using the reverse phase HPLC method of Example 2, and (+) denotes the presence of compound other than the CB2RA composition where the percentage composition was below levels of quantification of the reverse phase HPLC method. A first compound with a peak at 17.4 minutes is present in the amount from 0.1% to 0.6%, a second compound with a peak at 18.1 minutes is present in the amount of 0.01% to 0.2%, a third compound with a peak at 18.4 minutes is present in the amount of 0.2% to 0.7%, a fourth compound with a peak at 19.2 minutes is present in the amount of 0.01 to 0.15%, a sixth compound with a peak at 20.2 minutes is present in the amount of 0.05% to 0.15%, and a seventh compound with a peak at 23.2 minutes is present in the amount of 0.01% to 0.1%.

### Example 10. Preparation of a Lyophilized Cake of a CB2RA Composition

This example provides an exemplary lyophilized cake of a CB2RA Composition with PCP-PLA and trehalose that can be used, for example, in a tablet of capsule or reconstituted for injection or a topical administration.

PVP-PLA block copolymer (2.45 g) is dissolved in ethanol (12.25 mL) with magnetic stirring for 10 minutes. CB2RA Composition (50 mg) is then dissolved in ethanol (10 mL) in a glass vial with magnetic stirring and mixed directly with the polymer solution at room temperature. A further amount of ethanol (10.25 mL) is then added to the CB2RA Composition/polymer solution followed by dropwise addition of aqueous solution of trehalose (100 mg/mL, 17.5 mL). The resulting clear solution is left under stirring for 30 minutes at room temperature. Ethanol is removed by centrifugal evaporation and water is added to the resulting mixture to final CB2RA Composition concentration of 4 g/L. The bulk solution is then filtered using a 0.2 μm filter and transferred into 10 mL glass vials. Vials are then lyophilized using a VirTis Genesis 25EL lyophilizer. The composition of the exemplary lyophilized cake that can be produced by this method is shown in Table 14.

TABLE 14

| Ingredients | % (wt) |
|---|---|
| CB2RA Composition | 1.2% |
| PVP-PLA copolymer | 57.6% |
| Trehalose | 41.2% |
| Total | 100% |

### Example 11. Spray Dried CB2RA Composition

This example provides an exemplary spray dried composition of the composition of Example 10 that can be used, for example, in a tablet or capsule, a powder of inhalation.

PVP-PLA block copolymer, CB2RA Composition and Trehalose solution can be prepared as described in Example 10. Solvents (ethanol and water) are removed by spray drying using a Buchi B-290 spray dryer. The composition of the resulting powder is expected to be the same as the composition set forth in Table 14.

### Example 12. Lyophilized Cake of CB2RA Composition: Cyclodextrin

This example provides an exemplary lyophized cake of a CB2RA Composition with cyclodextrin that can be used, for example, in a tablet of capsule or reconstituted for injection or a topical administration.

Hydroxypropyl-β-cyclodextrin (HPβCD, 975 mg) is mixed thoroughly with CB2RA Composition (25 mg) in the presence of ethanol. Ethanol is evaporated and water (10 mL) is added slowly to the mixture under vigorous stirring. The resulting solution is left under stirring for 30 minutes at room temperature followed by filtration using a 0.2 μm filter. The filtered solution is transferred into 10 mL glass vial and lyophilised. The expected composition of the freeze-dried cake is given in Table 15.

TABLE 15

| Ingredients | % (wt) |
|---|---|
| CB2RA Composition | 2.5% |
| Hydroxypropyl-β-cyclodextrin | 97.5% |
| Total | 100% |

### Example 13. Topical Formulation

This example provides an exemplary aqueous formulation for topical application, for example, for topical application to the eye.

Polysorbate 80 (900 mg) is mixed with CB2RA Composition (100 mg) followed by addition of mannitol (5 g). To this mixture 94 g of water containing benzalkonium chloride (6 mg) is slowly added. Finally, the resulting solution is filtered through 0.2 μm filter. The expected composition of the resulting solution is given in Table 16.

TABLE 16

| Ingredients | % |
|---|---|
| CB2RA Composition | 0.1% |
| Polysorbate 80 | 0.9% |
| Mannitol | 5% |
| Benzalkonium chloride | 0.006% |
| Water | 94% |
| Total | 100% |

### Example 14. Topical Formulation

This example provides an exemplary aqueous formulation for topical application, for example, for topical application to the eye.

Polysorbate 80 (900 mg) is mixed with CB2RA Composition (100 mg). To this mixture 95.5 g of water containing mannitol (3 g), hydroxypropylmethyl cellulose (500 mg) and benzalkonium chloride (6 mg) is slowly added. Finally, the resulting solution is filtered through 0.2 μm filter. The expected composition of the resulting solution is given in Table 17.

TABLE 17

| Ingredients | % |
|---|---|
| CB2RA Composition | 0.1% |
| Polysorbate 80 | 0.9% |
| Mannitol | 3% |
| Benzalkonium chloride | 0.006% |
| Hydroxypropylmethyl cellulose | 0.5% |
| Water | 95.5% |
| Total | 100% |

### Example 15. Preparation of Ophthalmic Gel

This example provides an exemplary gel formulation for topical application, for example, topical application to the eye.

100 mg of CB2RA Composition is mixed with 2 g of PEG300 at room temperature to form a mixture. Carbopol 934 (2 g) is suspended in water (95.9 g). The mixture is added slowly to the aqueous solution containing Carbopol under vigorous mixing, forming colloidal gel-like dispersion. The expected composition of the formulation is given in Table 18.

TABLE 18

| Ingredients | % |
|---|---|
| CB2RA Composition | 0.1% |
| Carbopol 934 | 2.0% |
| PEG300 | 2.0% |
| Water | 95.9% |
| Total | 100% |

### Example 16. Ophthalmic Ointment

This example provides an exemplary ointment for topical application, for example, topical application to the eye.

CB2RA Composition (250 mg) is suspended in 2 g of mineral oil followed by mixing with white petrolatum (97.75 g). The expected composition of the preparation is shown in Table 19.

TABLE 19

| Ingredients | % |
|---|---|
| CB2RA Composition | 0.25% |
| Mineral oil | 2.00% |
| White petrolatum | 97.75% |
| Total | 100% |

### Example 17. Oral Controlled Release Formulation

This example provides an exemplary oral controlled release dosage form.

An exemplary process for the producing controlled release tablets consists of de-agglomeration, blending, compression and tablet esthetical coating.

The expected composition of the exemplary formulation is shown in Table 20.

Manufacturing operations are executed as follows. Xanthan gum is first blended together with colloidal silicon dioxide for 2 minutes, de-agglomeration is performed through a Comil followed by de-agglomeration of all remaining ingredients through a Comil. The mixture is then charged into V blender with Kollidon SR, CB2RA Composition, Xanthan Gum/colloidal silicon dioxide and Amyloflex. Blending is performed for 10 minutes. Next, magnesium stearate is added and the blending continued for 1 minute. The tablets are compressed using the tooling 9/32-inch, with round flat beveled edge and coated using Opadry coating dispersion at 15% solid content.

In order to improve the physical characterization of the blend, granulated CB2RA Composition is produced by wet granulation process using High shear blender. The process consists of preparing a dry blend of CB2RA Composition and Amyloflex for 5 minutes, wet massing by adding 35% of water, kneading for 1 minute, and drying the final product.

TABLE 20

| Ingredients | % |
|---|---|
| CB2RA Composition | 24.6% |
| Amyloflex ® | 17.8% |
| Kollidon SR | 36.4% |
| Xantan gum | 18.2% |
| Magnesium stearate | 0.5% |
| Colloidal silicon dioxide | 0.5% |
| Opadry II, Yellow 85F92421 | 2% |
| Total | 100% |

### Example 18. Oral Controlled Release Formulation

This example provides another exemplary oral controlled release dosage form.

The process consists of de-agglomeration, blending, compression and tablet esthetical coating. Manufacturing is executed as follows. First, Amyloflex and colloidal silicon dioxide are blended for 2 minutes followed by de-agglomeration through a Comil. De-agglomeration of all remaining ingredients through a Comil is performed followed by charging of the mixture into V blender, adding hypromellose and CB2RA Composition, and blending for 5 minutes. Next, sodium stearyl fumarate is added and the blending continued for 1 minute. The tablets are compressed using the tooling caplet or round flat beveled edge and coated using Opadry coating dispersion at 15% solid content. The expected composition of the exemplary formulation is shown in Table 21.

TABLE 21

| Ingredients | % |
|---|---|
| CB2RA Composition | 27.3% |
| Amyloflex ® | 46.0% |
| Hypromellose (HPMC K100M) | 23.0% |
| Colloidal silicon dioxide | 0.5% |
| Sodium stearyl fumarate | 1.5% |
| Opadry II, white | 1.7% |
| Total | 100% |

### Example 19. Oral Immediate Release Formulation

This example provides an exemplary oral immediate release formulation.

The process consists of de-agglomeration, blending, compression and tablet esthetical coating. Manufacturing operations are executed as follows. First, a portion of the MCC PH102 is blended with colloidal silicon dioxide for 2 minutes followed by de-agglomeration. De-agglomeration of remaining ingredients through a Comil is executed. Then, the mixture is charged into V blender together with CB2RA Composition, Explotab, and remaining MCC and is blended for 5 minutes. Magnesium stearate is added next with blending for 1 minute. The tablets are compressed using the tooling caplet or round flat beveled edge and coated using Opadry coating dispersion at 15% solid content. The expected composition of formulation is shown in Table 22.

TABLE 22

| Ingredients | % |
|---|---|
| CB2RA Composition | 40.0% |
| MCC PH102 | 51.5% |
| EXPLOTAB ® - sodium starch glycolate | 5.0% |
| Colloidal silicon dioxide | 0.5% |
| Magnesium stearate | 1.0% |
| Opadry II, white | 2.0% |
| Total | 100% |

### Example 20. Oral Immediate Release Formulation

This example provides an exemplary oral immediate release formulation.

The process consists of de-agglomeration, blending, compression and tablet esthetical coating. A portion of MCC PH102 and colloidal silicon dioxide is blended for 2 minutes followed by de-agglomeration. De-agglomeration of remaining ingredients through a Comil is performed. The mixture is then charged into V blender together with CB2RA Composition, Explotab, the remaining MCC, and starch 1500. Blending is performed for 5 minutes. Magnesium stearate is added with continued blending for 1 minute. The tablets are compressed using the tooling caplet or round flat beveled edge and coated using Opadry coating dispersion at 15% solid content, and the expected composition is set forth in Table 23.

TABLE 23

| Ingredients | % |
|---|---|
| CB2RA Composition | 35.0% |
| MCC PH102 | 48.5% |
| Starch 1500 | 10.0% |
| EXPLOTAB ® - sodium starch glycolate | 3.0% |
| Colloidal silicon dioxide | 0.5% |
| Magnesium stearate | 1.0% |
| Opadry II, white | 2.0% |

### Example 21. Immediate and Delayed Release Formulation (Pellets in Capsules)

This example provides an exemplary oral, immediate and delayed release formulation.

The manufacturing process consists of the production of immediate release (IR) pellets and delayed release (DR) pellets by fluid bed coating process and encapsulation.

Manufacturing operations are executed as follows. Immediate release pellets are produced as follows. A first active coating solution is prepared by dispersion of hypromellose in water, adding the API and mixing until complete dissolution. Drug layering of active coating solution on sugar spheres is performed next, followed by esthetical coating with Opadry dispersion at 15% solid content.

A portion of the immediate release pellets is coated with delayed release dispersion polymer and then coated with Opadry dispersion at 15% solid content. The expected composition of formulation is shown in Table 24.

TABLE 24
Double Pulsed Delivery Formulation

| | |
|---|---|
| Ingredients | % |

Immediate release pellets

| | |
|---|---|
| CB2RA Composition | 3.95 |
| Sugar spheres (30/35 mesh) | 36.35 |
| Hypromellose E5 | 0.21 |
| Opadry II white | 1.00 |

Delayed release pellets

| | |
|---|---|
| IR pellets | 41.51 |
| Eudragit L30 D 55 (dry basis) | 11.84 |
| Talc | 2.96 |
| Triethyl citrate | 1.18 |
| Opadry II white | 1.00 |
| Total | 100% |

### Example 22. Powder for Capsule

This example provides an exemplary powder for incorporation into a capsule.

The manufacturing process consists of the following steps: de-agglomeration, blending, and encapsulation using capsule filling unit.

The CB2RA Composition is blended with MCC PH301 for 5 minutes, followed by de-agglomeration through a Comil. Then, de-agglomeration of magnesium stearate through a Comil is performed. The magnesium stearate is added to the API blend into V blender and the blending continued for 1 minute. The powder is filled into capsules using capsule filling unit. The expected composition of formulation is shown in Table 25.

TABLE 25

| Ingredients | % |
|---|---|
| CB2RA Composition | 10.0 |
| MCC PH301 | 89.5 |
| Magnesium stearate | 0.5 |
| Total | 100% |

### Example 23. Celecoxib/CB2RA Composition Immediate Release Tablets

This example provides an exemplary combination product containing a combination of a CB2RA Composition and celecoxib in an oral, immediate release dosage form.

The process performed consists of de-agglomeration, blending, and compression. First, a portion of MCC PH102 is blended with colloidal silicon dioxide, Celecoxib and CB2RA Composition for 2 minutes, followed by de-agglomeration of the mixture through a Comil. The de-agglomerated blend is then charged into a V-blender. The remaining portion of MCC is de-agglomerated through a Comil, charged into V blender and blended with the previously de-agglomerated blend for 5 minutes. Next, magnesium stearate is added into the V-blender blended for 1 minute. Finally, the tablets are compressed using the tooling caplet or round flat beveled edge. The expected composition of the formulation is shown in Table 26.

TABLE 26

| Ingredients | % |
|---|---|
| CB2RA Composition | 2.0 |
| Celecoxib | 60.0 |
| MCC PH102 | 36.5 |
| Colloidal silicon dioxide | 0.5 |
| Magnesium stearate | 1.0 |
| Total | 100% |

### Example 24. Evaluation of CB2RA Formulation in Pre-clinical Model of Corneal Hyperalgesia

The cornea is a thin tissue at the front of eye that is densely innervated with sensory nerve endings. Damage to the nerve endings resulting, for example, from surgery, disease, or infection, can develop into corneal neuropathic pain an inflammatory response that involves leukocyte infiltration into the tissues of the cornea. This example provides a demonstration that analgesic and anti-inflammatory effects are produced by a pharmaceutical CB2RA composition formulation in a murine model of corneal hyperalgesia, which is representative of pain and inflammation that can result from surgery and other traumatic insults to the cornea. The ability of the CB2RA composition formulation, at two different concentrations, to inhibit the pain and inflammation induced by the model was compared to that of a sample of HU-308 formulated in soybean oil as described in Thapa (2018) supra.

Four groups of 4 male BALB/c mice (20-30 g) were used for the experiments. In a first step corneal injury was induced in each group as described by Thapa et al. (2018) supra. Mice were first anesthetized using 2-3% isoflurane. The center of the cornea on both eyes was then cauterized with silver nitrate using a micro-applicator brush. The brush was held in contact with the cornea for 2 seconds, producing a distinct superficial lesion of 2 mm, injuring the epithelial cell layer only, as occurs in certain human corneal surgeries (see, e.g., Mohammadpour et al. (2018) J. CURR. OPHTHALMOL. 30(2): 110-124). The cauterized eyes were then rinsed with saline and an ocular lubricant was applied if the animal appeared stressed.

A pharmaceutical formulation comprising a CB2RA composition (weight ratio of E1 to E2 of 98.8:1.2) was prepared using the general procedure described in FIG. 3, the final composition of which is set forth in Table 27.

TABLE 27
Composition of CB2RA Formulation

| | | Amount/Vial by |
|---|---|---|
| Ingredients | mg/vial | weight (%) |
| CB2RA Composition | 5.0 | 1.9% |
| PVP-PLA copolymer | 245.0 | 93.8% |
| Disodium dihydrogen phosphate | 9.6 | 3.7% |
| Sodium dihydrogen phosphate | 1.49 | 0.6% |
| Total | 261.09 | 100% |

After lyophilization, samples of the CB2RA composition formulation were reconstituted with water to a final concentration with respect to the CB2RA composition of either 0.25% or 0.5%. A vehicle control sample comprising PVP-PLA alone dissolved in water was also prepared.

One of the two pharmaceutical CB2RA composition concentrations, or vehicle, were then administered topically to the pre-cauterized eyes of the animals in groups 1-3 (5 μL/eye) at 30, 60, and 120 minutes post-cauterization. A separate group of animals (group 4) received a 5 μL dose of the 0.5% CB2RA composition sample at 30 min, 35 min, 60 min, 65 min and 120 min and 125 min, i.e. twice the dose of CB2RA composition received by animals in group 3. Table 28 summarizes the doses received by the various groups.

TABLE 28
Doses Administered

| | | Dosing (min post | Total |
|---|---|---|---|
| Groups | Test Agent | cauterization) | Vol. admin. |
| 1 | Vehicle | 30, 60, 120 | 15 μL |
| 2 | 0.25% CB2RA | 30, 60, 120 | 15 μL |
| 3 | 0.5% CB2RA | 30, 60, 120 | 15 μL |
| 4 | 0.5% CB2RA | 30, 35, 60, 65, 120, 125 | 30 μL |

Six hours following corneal cauterization, 1×5 μL of 1 μM capsaicin was administered topically to each cauterized eye in order to induce a pain response. The behavior of each animal (the number of blinks, squints and eye wipes made by the animals) was recorded on video for 30 seconds immediately following each application of capsaicin to generate a pain score for each animal. Individual animals in each treatment group were coded and experimental data were analyzed blinded. The average pain score for each group of animals was then plotted against the test article and volume received as shown in FIG. 20.

Twelve hours following corneal cauterization, the eyes of animals from group 3 and group 1 were harvested and fixed in 4% paraformaldehyde, followed by incubation in a 30% sucrose solution overnight. Corneal sections were cut using a cryostat, washed in PBS and blocked for non-specific binding for 2 hours (10% normal goat serum in 0.5% Triton-X/PBS), followed by a 48 hours incubation with an anti-Ly-6G primary antibody (Abcam, Cambridge. MA). Ly-6G is a glycosylphosphatidylinositol-anchor protein expressed predominantly on neutrophils and the anti-Ly-6G antibody allows for detection of these cells in the sections. The sections were then washed with PBS and incubated with a secondary antibody, goat anti-rat Alexa Fluor® 488 to allow neutrophil visualization. Stained sections were washed in PBS and mounted on slides.

The number of neutrophils in the corneal sections, indicative of the degree of inflammation induced in the eyes, was quantified in corneal sections at 20× magnification using a Zeiss Axiovert 200 M microscope. Three representative images were taken from each section of the right and left corneal peripheries and from the center of the cornea, respectively. The total number of neutrophils from these three images was counted for each section and summed to represent the total neutrophil number for a single corneal section. A total of five sections with 120 μm intervals from each eye were analyzed and the neutrophil number was averaged.

Based on the pain scores from the video recordings revealed the vehicle treatment produced an average pain score of 34, whereas the administration of CB2RA formulation at 0.25 and 0.5% produced significant reductions in pain scores (12, and 18, respectively; see FIG. 20) with the reduction in pain score being inversely proportional to CB2RA concentration. When compared to the results from Thapa et al. (2018) supra for HU-308 administered where a 1.5% solution of the CB2 agonist generated a pain score of was 17 (see, 1.5% HU-308 in FIG. 20). Doses of Hu-308 lower than 1.5% failed to generate an analgesic effect. The results generated using the same model indicate that CB2RA delivered as a PVP-PLA formulation is at least three times more effective in reducing the pain score in corneal hyperalgesia model when compared to HU-308 formulated in soybean oil. Surprisingly animals in group 4 that received twice the CB2RA dose (total 30 μL) had higher pain scores that those that only received 15 μL of the composition.

Neutrophil infiltration into the cornea following treatment with 0.5% CB2RA formulation is shown in FIG. 21. Topical administration of 0.5% CB2RA formulation significantly reduced neutrophil number (neutrophils/section=101) compared to vehicle-treated eyes (neutrophils/section=214). The level of neutrophil reduction observed for 0.5% CB2RA formulation was similar to that observed for HU-308 solution at three times higher concentration (data extracted from Thapa (2018) supra), confirming greater anti-inflammatory effect of CB2RA formulation as compared to that of HU-308 formulation. Together these data demonstrate that the CB2RA composition comprising 98.8% HU-308 and 1.2% HU-433, delivered as micellar PVP-PLA formulation, provides a greater analgesic and anti-inflammatory effect than HU-308 alone when delivered in soybean oil.

### Example 25. Further Evaluation of CB2RA Formulation in Pre-clinical Model of Corneal Hyperalgesia

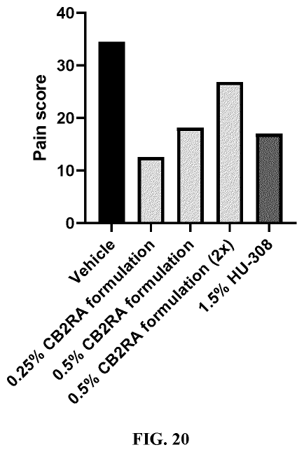

With reference to Example 25 and FIG. 20, additional studies were performed to assess the analgesic effect of the CB2RA Formulation at concentrations lower than had been tested previously these being, with respect to the CB2RA Composition, concentrations of 0.075% wt/vol. 0.25% wt/vol and 0.125% wt/vol. Table 29 displays the pain scores recorded 6 h post cauterization for all doses tested (including the new doses) while FIG. 22 presents these data graphically.

TABLE 29
Pain Scores After Topical Administration
of the CB2RA Formulation

6 h Post Cauterization

| Group | Test Agent | Pain Score | SEM |
|---|---|---|---|
| 1 | Vehicle | 34.5 | 3.9 |
| 2 | P2005 (0.075% TA-A001) | 20.0 | 5.8 |
| 3 | P2005 (0.125% TA-A001) | 13.4 | 5.5 |
| 4 | P2005 (0.25% TA-A001) | 12.5 | 2.2 |
| 5 | P2005 (0.5% TA-A001) | 18.1 | 3.5 |

As FIG. 22 shows the pain scores produced by the different doses of the CB2RA composition exhibited an apparent U-shaped curve with doses from 0.075% to 0.25% generating increasing levels of analgesia and the 0.5% dose, while significantly more potent than vehicle, appearing less analgesic, than for example the 0.25% dose.

This effect was unexpected but speaks to the possibility of a defined therapeutic window for the CB2RA Composition. As no adverse effects (eye reddening or other signs of irritation) were observed for any dose this observation cannot be extended to comments on the therapeutic index however. Thus, while it has been reported that other cannabinoid receptor agonists have a narrow therapeutic index, this does not seem to be the case for the CB2RA Composition of the present invention; tolerability is not compromised by increasing doses of the formulation.

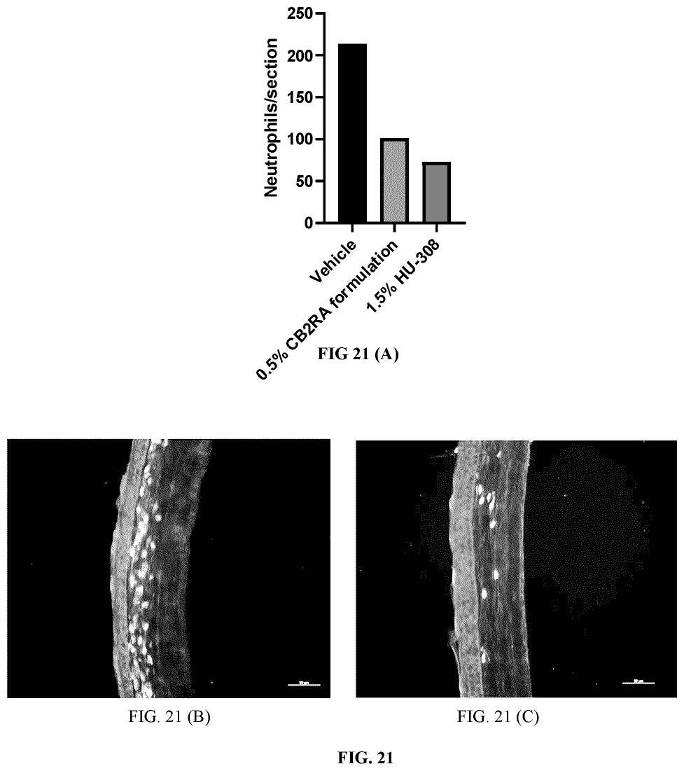

These data were best fitted using a cubic spline function (see FIG. 23) which predicted a maximum effective dose in this model of approximately 0.18% wt/vol and an ED50, based on values between the vehicle (zero response) and the maximum effective dose, of 0.06%. This latter figure is significantly lower than those generated by Thapa et al (2018), supra, for A8 THC (ED50; 1%) or CBD (ED50; 5%) which laboratory also generated the present results.

### Example 26. Evaluation of COX Formulation in Pre-clinical Model of Corneal Hyperalgesia

With reference to Examples 25 and 26, additional studies were performed to compare the effects of the CB2RA Formulation and the NSAID-based COX Formulation 1 in the corneal hyperalgesia murine model. Experimental methods employed were the same as those described in detail in Example 25.

COX Formulation 1 was prepared as follows. 450 mg of PVP-PLA block copolymer was dissolved in 1 mL of water for injection under magnetic stirring for approximately. 10 minutes. 0.4 mL of a 0.1N aqueous solution of 0.1 NaOH was then added to the polymer solution, under stirring to bring the pH to approximately 7.5. Next, 0.25 mL of 100 mM sodium phosphate buffer pH 7.0 was added to the solution followed by the addition of 1.25 mL of aqueous solution of mannitol at concentration 100 mg/mL. Next, 50 mg of celecoxib was dissolved in 1 mL of ethanol in glass vial under magnetic stirring at room temperature and added drop by drop to polymer solution over approximately. 1 min. The resulting clear solution was then cooled in an ice bath and then placed in a cold chamber at 6° C. under stirring for approximately 20 min. After removing the sample from a cold chamber, 1.10 mL of water was added to the formulation. Next, the solution was concentrated to 25% of its initial weight under reduced pressure in a Rocket centrifugal evaporator. 3.6 mL of water was then added to the concentrated solution, to obtain the formulation with a final CEL concentration 10 mg/mL. The formulation was then filtered through a 0.2 um Nylon Target2 filter (Thermo Scientific). Filtered formulation was transferred into 10 mL glass vial and freeze-dried using a VirTis Genesis 25EL lyophilizer. The composition of the resulting lyophilized celecoxib cakes (the COX Formulation 1) is shown in Table 30.

TABLE 30
Composition of Freeze-dried COX Formulation 1

| Ingredients | mg/vial | %/vial |
|---|---|---|
| Celecoxib | 50.0 | 7.8% |
| PVP-PLA copolymer WB-4 | 450.0 | 70.0% |
| Mannitol | 125 | 19.4% |
| Sodium hydroxide | 1.6 | 0.2% |
| Sodium phosphate monobasic | 7.5 | 1.2% |
| Sodium phosphate dibasic | 8.9 | 1.4% |
| Total | 643 | 100% |

Lyophilized cakes of the COX Formulation 1 were reconstituted in in water for injection generating clear, particle-free solutions with a celecoxib concentration of 25 mg/mL. The pH of the reconstituted cakes was in the range from 6.8 to 7.2 as measured using an Accumet AP61 pH-meter equipped with a gel-filled epoxy-body combination electrode. Optical transmittance was determined in 1-cm disposable polystyrene cuvettes on an Agilent Cary UV-Vis-NIR 5000 spectrometer. The measurements were performed at 650 nm and room temperature using empty cuvette as a blank. The reconstituted mixtures had optical transmittance of above 90%. The osmolality of the reconstituted samples was measured with a freezing point depression 3300 Micro-Osmometer (Advanced Instruments) and shown to be between 420 to 450 mOsm/kg for the various samples tested. The Z-average (mean size) of the micelles in solution and their size distribution were determined at 25° C. by dynamic light scattering using a Malvern Zetasizer Nano ZS equipped with 10 mW He—Ne laser operating at 633 nm. Z-average and polydispersity varied from 47 to 52 nm and from 0.38 to 0.45, respectively.

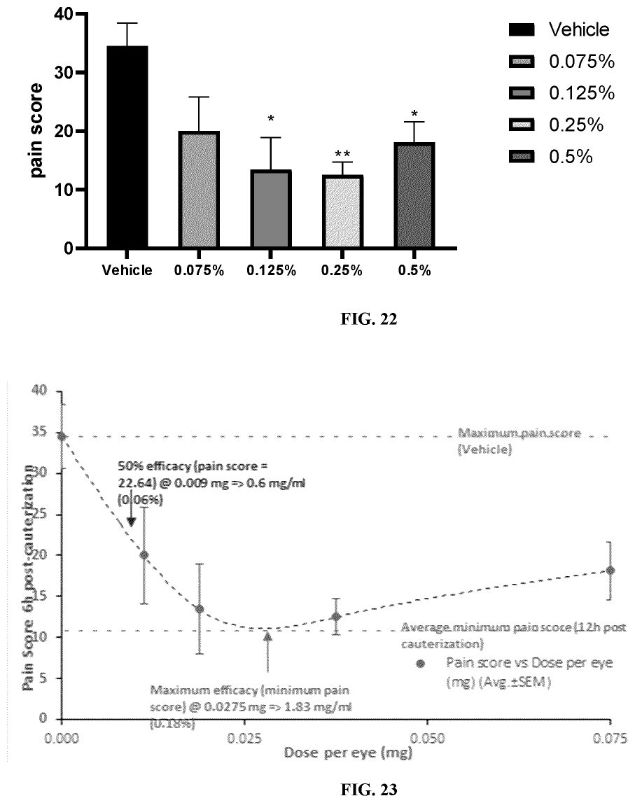

The ability of the COX-Formulation 1 to reduce ocular pain in the corneal hyperalgesia model of Examples 25 and 26 was assessed at two concentrations these being a 1.5% wt/vol solution (with respect to celecoxib) and a 3% wt/vol solution (again with respect to celecoxib). The higher drug concentrations employed for the celecoxib formulation reflected the approximately 10-fold differences in potency seen between celecoxib and the CB2RA composition in rodent hind paw incisional pain models (Example 7). As may be seen from FIG. 24, while the higher dose of celecoxib generated a lower pain score than the lower dose (viz. a potential trend), neither dose produced a pain score that was statistically different from the value determined for vehicle, values recorded being 40±2 for the 1.5% dose and 24±3, for the 3% dose which are both much higher than those for the CB2RA Composition confirming the greater analgesic potency of the CB2RA Composition reported in Example 7. Pain scores displayed by application of vehicle to the test animals in this study were highly consistent with those from previous experiments (average pain score of 32±3) thereby allowing this comparison of results.

These data are important with respect to potential uses of the CB2RA Composition and Formulation in the treatment of corneal pain because NSAID drugs inhibiting COX-1 and COX-2 enzymes such as celecoxib (Gilad Rimon, Ranjinder S. Sidhu, D. Adam Lauver, Jullia Y. Lee, Narayan P. Sharma, Chong Yuan, Ryan A. Frieler, Raymond C. Trievel, Benedict R. Lucchesi, William L. Smith. Coxibs interfere with the action of aspirin by binding tightly to one monomer of cyclooxygenase-1) and ketorolac currently represent ‘standard of care analgesics’ for corneal surgery (https://www.reviewofoptometry.com/article/topical-nsaid-update). These data support the use of the CB2RA Composition and Formulation for corneal surgery at lower doses than can be achieved for NSAIDs avoiding NSAID side-effects such as delayed wound healing and corneal thinning (Iwamoto, S., Koga, T., Ohba, M. et al. Non-steroidal anti-inflammatory drug delays corneal wound healing by reducing production of 12-hydroxyheptadecatrienoic acid, a ligand for leukotriene B4 receptor 2. Sci Rep 7, 13267 (2017). https://doi.org/10.1038/s41598-017-13122-8.)

### Example 27. Effects of the CR2RA Composition and CB2RA Formulation in Models of Ocular Inflammation), alone and together with COX Formulation 1

Many pathological conditions of the eye are associated with inflammation or have inflammation as an underlying cause. Thus, it is is well recognized that chronic dry eye disease involves cycles of inflammation within the cornea and conjunctiva for example (Baudouin C, Irkeg M, Messmer E M, et al. Clinical impact of inflammation in dry eye disease: proceedings of the ODISSEY group meeting. Acta Ophthalmol. 2018; 96(2):111-119. doi:10.1111/aos.13436; Yamaguchi, T. (2018). Inflammatory Response in Dry Eye. Investigative Ophthalmology & Visual Science, 59(14), DES192. doi:10.1167/iovs.17-23651) and that anti-inflammatory agents such as cyclosporin and corticosteroids can alleviate symptoms (Hessen M, Akpek E K. Dry eye: an inflammatory ocular disease. J Ophthalmic Vis Res. 2014; 9(2):240-250). The CB2RA Composition has been shown to reduce corneal inflammation as described in Example 26. The ability of the CB2RA Formulation to deliver the CB2RA Composition throughout the Anterior segment of the eye (Example 30) also supports its value in treating uveal inflammation diseases such as uveitis which can be blinding.

Similarly retinal inflammation is associated with a number of blinding diseases such as the diabetic retinopathies (Chung Y R, Kim Y H, Ha S J, et al. Role of Inflammation in Classification of Diabetic Macular Edema by Optical Coherence Tomography. J Diabetes Res. 2019; 2019:8164250. Published 2019 Dec. 20. doi:10.1155/2019/8164250) and autoimmune conditions such as Autoimmune retinopathy a rare disease causing retinal degeneration. Age-related macular degeneration (AMD) is another inflammatory retinal disease. AMD is a leading cause of blindness globally with an estimated 11 million North Americans being afflicted by the disease. As the name suggests its prevalence steadily increases with age affecting 2% of the population at the age 40, but over twenty five percent by the time they are 80. AMD may be categorized as dry AMD, or wet AMD;

Dry AMD: Although the precise aetiology of dry AMD is not well understood, low grade chronic retinal inflammation is thought to be a major driver of the disease. Thus, the photoreceptors and retinal pigment epithelial (RPE) cells of the retina display high respiration rates requiring high local oxygen concentrations if they are to function efficiently. A consequence of this oxygen rich environment however, when coupled to the highly oxidative conditions of focused light exposure, is the prolific generation of reactive oxygen species (RoS). While short lived, RoS are highly destructive to the proteins, lipids and cholesterol found in high concentration in RPE cells and adjacent Bruch's membrane (BM) and it is damaged and transformed forms of these molecules (including pro-inflammatory 7-ketocholesterol, complement factors and other cellular debris) that comprise the drusen deposits typifying the disease.

Patients with dry AIMD may be classified as having early, intermediate, or late-stage disease based on the nature of the drusen observed in their macula. In early to intermediate dry AMD, drusen appear as small (60 μm-130 μm) yellowish brown deposits. However, as the disease progresses so drusen grow reaching to up to 1000 μm in isolated areas. Such larger drusen result in death of the proximal RPE cells and photoreceptors above them, vision being progressively compromised by the process. Should separate areas of cell death become confluent and impinge on the central foveola, as occurs in late-stage dry AMD (aka geographic atrophy), then vision loss is severe.

Progression from small to ever larger drusen is driven by chronic retinal inflammation engendered by the pro-inflammatory drusen components which in turn causes attraction and accumulation of monocytes/macrophages and other cells of the immune system (leukocytes) to the inflamed area. As leukocytes accumulate so the inflammatory response is heightened increasing RPE destruction which then leads to further drusen formation and deposits forming on both sides of the RPE. This ‘lipid barrier’ further inhibits RPE function and viability. There are currently no treatments for dry AMD, a major unmet need.

CB2 receptor agonists, including the CB2RA composition described herein, have been shown to have immunomodulatory effects. For example, they have been shown to attenuate the levels of pro-inflammatory mediators in models of corneal hyperalgesia, uveitis and vitreoretinopathy where they also reduced accumulation of the leukocytes that mediate chronic inflammation and tissue destruction. An ability to attenuate the immune response in the retina, mitigate immune cell accumulation and thereby control RPE destruction would be important for the treatment of dry AMD.

The ability of the CB2RA composition to reduce retinal inflammation was determined by measuring its ability to modulate the recruitment of activated monocytes to the subretinal space of mice following blue-light exposure. For these first proof-of-principle experiments the CB2RA formulation was administered to animals by intraperitoneal injections at doses of up to 3 mg/kg/day.

Briefly male C57BL/6 mice were exposed to blue light (425 nm) from a light-emitting diode (LED) at an illuminance intensity of 6000 lux for 4 days, without prior dark-adaptation. The mice were treated twice per day by intraperitoneal (IP) injection of saline, vehicle (PVP-PLA), or the CB2RA Formulation at dose of 0.5 mg/kg, 1.5 mg/kg, and 3 mg/kg with respect to the CB2RA composition. Treatments were administered from the first day of exposure until the end of blue-light exposure. During the illumination period, ophthalmic atropine solution 1% (Alcon, USA) was administered daily for pupil dilation. At the end of the illumination period, mice were maintained on a regular 12 h/12 h light/dark cycle at 300 lux for 3 days prior to sacrifice.

Enucleated eyes were then collected and immersed in phosphate buffered 4% paraformaldehyde (PFA) solution for 1 hr. The eye tissue was then permeabilized with phosphate buffer saline (PBS) containing 0.1% Triton-X-100 and 10% fetal bovine serum (FBS) and then incubated with anti-IBA-1 antibody (FujiFilm Wako Pure Chemical Corp., Japan) overnight in the same PBS buffer. IBA-1 is a macrophage-specific antibody that allows detection of the subretinal activated monocytes responsible for the inflammatory response: rhodamin-phalloidin (Abcam, Canada), a polymerized actin marker, was used as a counterstaining agent to visualize the RPE cells. After 30 min. the tissue was washed with PBS buffer and then incubated with a fluorochrome-coupled and anti- (anti-IBA-1) antibody (Alexafluor-488, Life Technologies, USA). Post this second incubation the tissue was washed three times for 5 min. with 0.1% Triton-X-100 in PBS, and then flatmounted for fluorescence microscopy analysis (Zeiss, Germany).

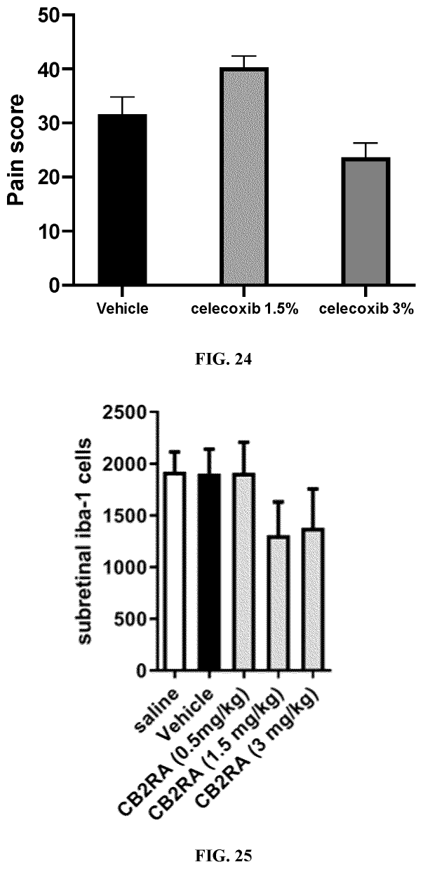

As may be seen in FIG. 25, while the 0.1% dose of the CB2RA Composition caused no reduction in monocytes numbers compared to the control treated animals, the number of IBA-1 positive monocytes in the subretinal space was greatly decreased when 3 and 6 mg/kg/day CB2RA Composition doses were administered supporting that the CBRA composition has an anti-inflammatory effect in the retina.

Wet AMD: Some patients with late or advanced stage AMD, in addition to geographic atrophy, will experience choroidal neovascularization (CNV), that is the formation of new immature, fragile and leaky choroidal blood vessels which break through the BM into the retina. When this occurs, the disease is termed neovascular, exudative, or “wet” AMD. Wet AMD causes more rapid progressive loss of vision than geographic atrophy and is experienced by 10-15% of AMD suffers.

Central to the aetiology of Wet AMD is Vascular Endothelial Growth Factor (VEGF). Under normal conditions low level changes in VEFG concentration are thought to maintain homeostatic control over the blood supply to the RPE via the so-called choriocapillaris. However, under the inflammatory conditions of AMD local overproduction of the cytokines IFN-γ, IL-1β and TNF-α results in upregulation of and over production of VEGF by activated leukocytes, inflamed fibroblasts and RPE cells. Such is increases then result in increased new blood vessel growth (angiogenesis) and increased vascular permeability (leakiness). It is the extent of the angiogenesis, and the leakiness of these new vessels, that controls the amount of vascular exudate in the retina and sub-retinal space, and therefore the degree of vision loss that occurs in Wet AMD. Excessive amounts of vessel exudate may also cause RPE and or retinal detachment resulting in blindness.

Modulation of pro-inflammatory cytokines by cytokine specific antibodies or, as more commonly occurs, modulation of ocular VEGF levels using anti-VEGF antibodies has been shown to reduce the progression of CNV in some patients with AMD supporting the central role of VEGF in wet AMD and an approach to effective treatment of the disease. However, such therapies are only beneficial in approximately 40% of eyes and often only for a limited period. Alternative approaches to treatment are required.

The CB2RA Composition and CB2RA Formulation described herein have been shown to reduce levels of pro-inflammatory cytokines and chemokines in the eye after topical administration (see Example 25) supporting the potential of CB2R agonism to reduce VEGF production and thereby angiogenesis. This potential was assessed using an ex vivo model which allows direct measurement of anti-angiogenic effects of test agents on choroidal vessel sprouting in choroid/RPE complexes. As the CB2RA composition had been shown in in vivo models to display synergistic anti-inflammatory effects with NSAIDs (celecoxib) the CB2RA composition was tested in the presence and absence of micellar COX Formulation 1.

Briefly, choroidal explants were obtained from 6-week-old mice and prepared according to the methods described by Tahiri et al. Thus, the enucleated eyes were first placed in a Petri dish containing 1× Hank's balanced salt solution (HBSS, VWR, Canada). Eyes were then opened under the ora serrata to remove the anterior structures of the lens and cornea, followed by careful removal of the neuroretina comprising the sclera/choroid/RPE complex. These preparations were cultured at 37° C. in 5% CO2 for 3 days in endothelial cell growth basal medium (EBM-2) supplemented with Microvascular Endothelial SingleQuots kit (EGM-2MV, Lonza Bioscience, Switzerland). The EBM-2 culture medium was changed on day 3 explants then being incubated with either; i) PBS; ii) anti-VEGF antibody (20 ng/mL, R&D Systems, USA); iii) the CB2RA Formulation (CB2RA concentrations: C1=0.1 μM, C2=1 μM, C3=5 μM and C4=10 μM); iv) celecoxib (COX Formulation 1; celecoxib concentrations: C1=1 μM, C2=10 M, C3=17.5 μM and C4 25 μM); v) a mixture of the CB2RA Formulation and COX Formulation 1 (CB2RA and celecoxib concentrations 5 μM and 17.5 μM, respectively) or; vi) PVP-PLA (Vehicle). Seven explants were used for each test article or control. Photographs of individual explants were taken before (T0) and 24 hrs after the test treatment (T24 h) using an Axiovert 200 M inverted microscope (Zeiss, Germany). The degree of neovascularization shown in the photographs (total vascular area T24 h minus total vascular area TO) as generated by each control or test article administration was determined using ImageJ software (NIH, USA).

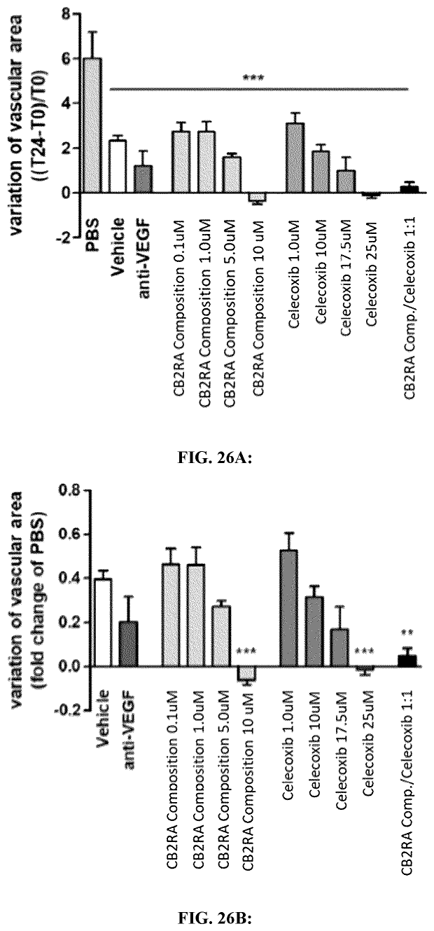

As may be seen from FIG. 26A the CB2RA composition, celecoxib and the PVP-PLA based vehicle displayed a significant anti-angiogenic effect compared to PBS with the CB2RA and the celecoxib test articles demonstrating this in a dose-dependent manner. When normalized to the PBS response (FIG. 26B) higher doses of both the CB2RA composition and celecoxib showed a significant difference compared to the PVP-PLA vehicles. At these concentrations, both compounds were able to reduce the vascular area with a better efficacy than anti-VEGF.

Synergy between the CB2RA Composition and celecoxib has been demonstrated in a model of acute incisional pain (Example 8) where the ED50 of both agents was reduced greatly when combining them, to an extent greater than simple summing. Here again a combination of the two active agents generated a strong anti-angiogenic effect that was greater than simple addition viz., when referring to FIG. 26B the vascular area relative to PBS for the CB2RA Composition tested at 1 μM was 0.27 while the area relative to PBS for the 17.5 μM celecoxib formulation was 0.17. A 1:1 mixture might therefore be expected to generate a vascular area relative to PBS of;

(0.27+0.17)/2=0.22 (i)

However, the value measured for the 1:1 combination of the CB2RA composition and celecoxib generated a vascular area relative to PBS of 0.04 which is some 5 times greater than might be expected again supporting synergy between the two molecules. This effect was again greater than that achieved by the anti-VEGF antibody tested in the same experiments. The CB2RA, the COX Formulation 1 and the combination of the CB2RA and an NSAID molecule may therefore be potential new treatments for the neovascularisation that occurs in AMD or other pathologies were new blood vessel growth is deleterious including in oncology.

### Example 28. Effect of CB2RA Formulation on Corneal Wound Healing in Rabbits

Regenerating ocular surface integrity is key to maintaining ocular homeostasis and long-term health. NSAID and corticosteroid drugs, used as analgesics after corneal surgery, are associated with reduced rates of wound healing and increases in intraocular pressure. The objective of the rabbit ocular surface study was to test whether the CB2RA Formulation, when applied topically to the eyes of rabbits, affected the rate of corneal wound healing and ocular surface integrity as indicated by fluorescein staining in the Algerbrush-induced corneal wound model. Further, it was performed to determine if intraocular pressure was increased by dosing with the CB2RA Formulation.

Thirteen New Zealand albino rabbits with body weight approximately 2.5 kg were used and maintained in accordance with the guidelines for the care and use of laboratory animals. Animals were assigned to three groups (vehicle, n=5, and CBR2RA at doses 0.25% and 0.5%, n=4) after baseline ocular examination (day −1). On day 0 of the study, rabbits received a drop of 0.5% proparacaine hydrochloride to both eyes following anesthesia with ketamine/xylazine. After 3-5 minutes, 5% betadine was applied to the eyes and rinsed with sterile saline. A speculum was used to keep the eye open for the duration of the procedure. For the right eye (OD), the cornea was scored gently with a 3 mm punch tool and the top layer of the epithelium was removed with an Algerbrush II rust ring removal tool within the area delineated by the punch tool. Following the procedure, buprenorphine was provided to alleviate pain and discomfort. This treatment is not expected to interfere with corneal wound healing. Animals did not express signs of pain or distress during the studies. Left eyes were untreated. At the completion of the study (day 3), all animals were euthanized with sodium pentobarbital at a dose level of 400 mg/kg administered by IV injection following intramuscular administration of 35 mg/kg ketamine/5 mg/kg xylazine under deep anesthesia.

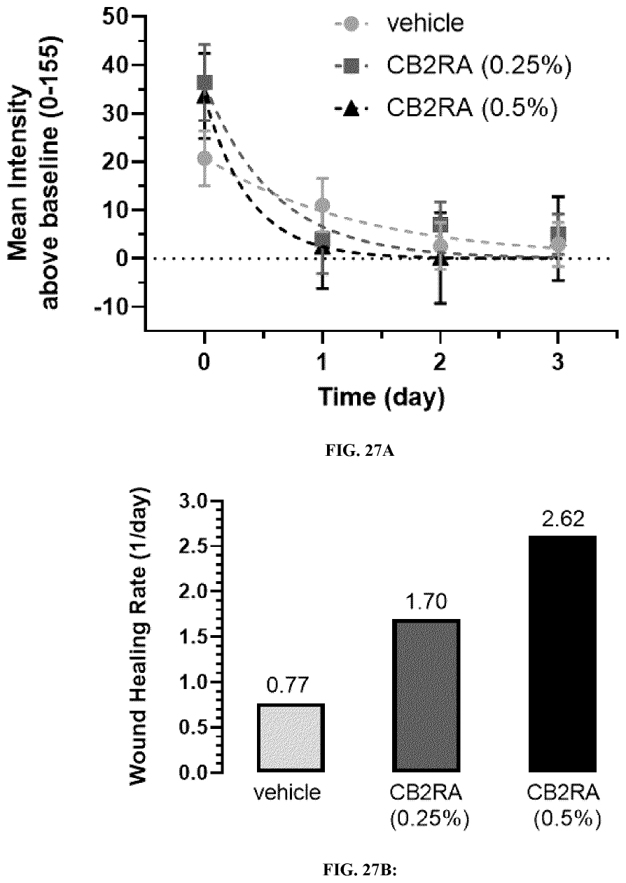

CB2RA Formulation or vehicle (50 μL) was carefully administered to the ocular surface (right eye) of the rabbit using a calibrated micropipette twice per day from day 0. Wound healing was examined by fluorescein staining on day −1 (baseline) and then once per day from day 0 to day 3. Briefly, animals received a 20 μL topical dose of 0.75% sodium fluorescein (FischerSci, USA) stain to the right eye (OD). The severity of the fluorescein stain was assessed from images taken using a Pictor Plus camera (Volk, USA). Following the completion of the study, the micrographs were analyzed for mean staining intensity in the 3 mm diameter lesion area using ImageJ software (NIH, USA). The rates at which CB2R Formulation effected wound healing (i.e., where fluorescent intensity return to baseline values pre-surgery) were then calculated (FIGS. 27A and 27B).

Briefly, as FIG. 27A shows, while fluorescent intensity remained above baseline for the three days of the study for the vehicle group, baseline line values were obtained when using the CB2R Formulation within 1 day for the 0.5% treated animals and within 2 days for the 0.25% treated animals. These results are expressed as healing rates in FIG. 27B. The test articles were deemed highly successful in healing the cornea with rates of healing displaying a dose response effect.

The intraocular pressure was measured before surgery and then prior to any other test on each subsequent day of the test. Measurements were recorded for both the left (untreated) and right (treated) eyes of the animals. All the values were are within the normal range for these animals (from 9.5 to 15.75 mmHg) throughout the study and there was no statistical difference in the pressures of treated and untreated eyes supporting the conclusions that the application of the CB2RA Formulation does not elevate intraocular pressure.

Taken together, the study demonstrated that, unlike NSAIDs and corticosteroids, the CB2RA Formulation increased the rate of corneal wound healing compared to control and had no effect on intraocular pressure.

### Example 29. CB2RA Composition Biodistribution and Control of Inflammation in a Rat Model of Corneal Hyperalgesia after Topical Application

Ocular diseases occur throughout the eye and it would therefore be of greater therapeutic benefit if a drug such as the CB2RA Composition as described herein could access all regions of the eye by simple topical administration as an eye drop. The ability of the PVP-PLA based CB2RA Formulation to deliver the CB2RA Composition to the front and the back of the eye after topical application was studied using a rat model of corneal hyperalgesia based on the mouse model developed by Thapa et al (Thapa D, Cairns E A, Szczesniak A M, Toguri J T, Caldwell M D, Kelly M E M. The Cannabinoids Δ⁸THC, CBD, and HU-308 Act via Distinct Receptors to Reduce Corneal Pain and Inflammation. Cannabis Cannabinoid Res. 2018 Feb. 1; 3(1):11-20. doi: 10.1089/can.2017.0041. PMID: 29450258; PMCID: PMC5812319) the larger eyes of the rat enabling more precise dissection and therefore more accurate measurement of the CB2RA Composition in the various tissues.

Briefly, the rats were profoundly anesthetized by isoflurane 2.54% inhalation and then a micro-applicator containing silver nitrate (Grafco 6″) was applied on the surface of their eyes for 2 seconds to cauterize a 1 mm diameter area. Eyes were then rinsed with saline and an ocular lubricant (Systane® Ultra) was applied to prevent drying.

For dosing, the rats were restrained and dosed topically with a 10 μl droplet of either saline, PVP-PLA polymer or CB2RA Formulation at 30 minutes, 1 hour and 2 hours post cauterization. The concentrations of the CB2RA Composition in the CB2RA Formulation applied were between 0.075% and 0.5%. Six hours after cauterization 5 μL of a 1 μM capsaicin solution was applied to the surface of each eye and after eight hours the animals were euthanized and all eyes enucleated.

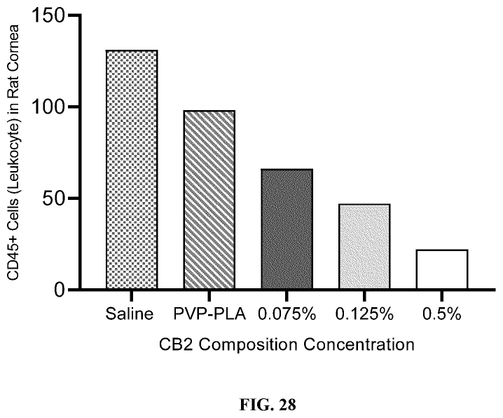

The left eye from each animal was dissected and fixed in paraformaldehyde 4% for 24 hours, followed by sucrose 20% for at least 24 hours. The dissected eyes were then embedded in OCT compound (Tissue Plus; Fisher) and cut in to 12 μm-thick sections using a Leica CM1950 cryostat. Non-specific protein binding was blocked with 5% bovine serum albumin for 1 hour after which the corneal sections were incubated overnight at 4° C. in the dark with an anti-CD45 antibody diluted 1:200 (Abcam, Clone MRC OX-1). The corneal sections were then washed with PBS and an anti-fade mounting media with DAPI (ProLong Glass Antifade Mountant with NucBlue Stain, Thermofisher) was applied. Whole slides with one to four corneal sections per slide were scanned with a digital slide scanner (Pannoramic MIDI II, 3DHISTECH). The number of leukocytes in the cornea was analyzed using the QuantCenter image analysis platform (3DHISTECH); the average number of leukocytes in the cornea after exposure to the different concentrations of the CB2RA Composition are shown in FIG. 28.

As FIG. 28 shows, the CB2RA composition reduced corneal inflammation in a dose dependent manner as reflected in the progressively lowers numbers of leukocytes that had accumulated in the tissue as the dose of the CB2RA Composition increased. These data confirm those generated in the mouse model that the CB2RA Composition displays an anti-inflammatory effect in corneal tissues after topical administration.

The right eyes of the rats euthanized 8 hrs after cauterization were enucleated and then dissected into front of eye (cornea, ciliary body and aqueous humour) and back of eye (vitreous humour, retina and choroid) segments. The concentration of the CB2RA Composition in each segment was then calculated as follows;

The dissected tissues were first ground in cold PBS buffer in CK14-2 mL vials with a Precellys® bead beater homogenizer. After grinding, cold ethyl acetate was added to extract the CB2RA Composition from the aqueous homogenate which extract was then vortex mixed for 15 seconds. Centrifugation was then performed at 13,000 rpm for 15 minutes at 4° C. and 150 μL of the supernatant was transferred in RSA vials for GC MS/MS analysis. All samples were kept at −80° C. prior to CB2RA Composition quantitation.

Samples were analyzed using an Agilent Technologies 7000C triple quadrupole mass spectrometer fitted with Agilent Technologies HP-5 ms Ultra-Inert, 30 m×0.32 mm×0.25 μm column with a helium flow rate of 2 mL/min and an inlet temperature of 325° C. A sample volume of 0.75 μL was used for each injection which was performed in splitless mode. The oven temperature ramp conditions are provided in Table 31.

TABLE 31
Oven Temperature Ramp Up Rate

| | Rate | Value | Hold time | Run time |
|---|---|---|---|---|
| Ramp | (° C.) | (° C.) | (min.) | (min.) |
| Initial | — | 150 | 1 | 1 |
| Ramp 1 | 30 | 320 | 10 | 16.667 |

The tandem mass spectroscopy scan segments used are provided in Table 32. The transfer line temperature used was 280° C. with a solvent delay of 4 minutes. Multi Reaction Monitoring (MRM) conditions were employed.

TABLE 32
Tandem Mass Spectrometry Scan Segments

| | | Pre- | | | | | |
|---|---|---|---|---|---|---|---|
| | Com- | cursor | MS1 | Product | MS1 | Dwell | Collision |
| # | pound | ion | Resolution | ion | Resolution | (ms) | (eV) |
| 1 | TA-A001 | 318 | Wide | 233 | Wide | 100 | 25 |
| 2 | TA-A001 | 318 | Wide | 219 | Wide | 100 | 25 |
| 3 | TA-A001 | 318 | Wide | 205 | Wide | 100 | 25 |
| 4 | TA-A001 | 318 | Wide | 191 | Wide | 100 | 25 |

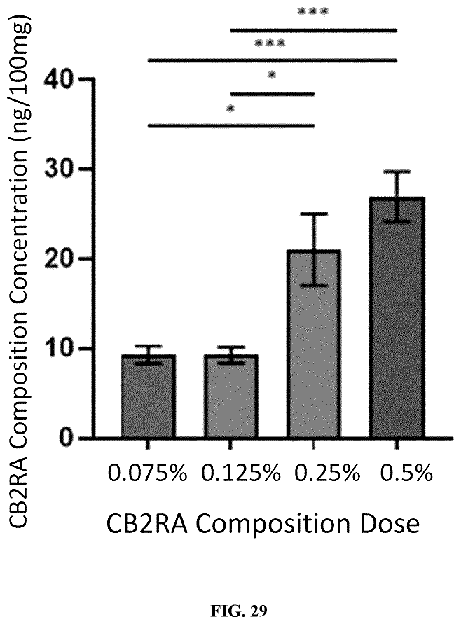

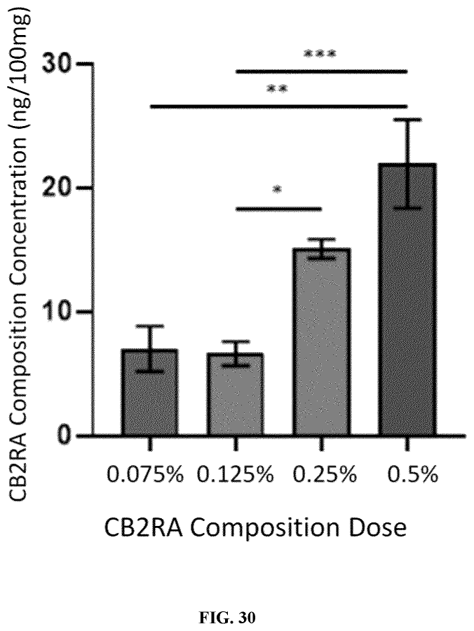

The amounts of the CB2RA Composition in the front and back segments of the eyes are presented in FIGS. 29 and 30.

As may be seen, significant concentrations of the CB2RA Composition were measured in both the front and the back of the eye some 6 hours after topical application of the formulation. No concentrations were measured when vehicle or saline were used as expected. All concentrations measured reflected the different amounts of the CB2RA Composition applied in both the back of eye and front of eye samples.

The continued presence of the CB2RA Composition 6 hours after administration of the drug, i.e. at a time where complete clearance from ocular tissues may have been expected supports the potential for the CB2RA Formulation to provide prolonged inflammation-suppressive effects in a dose/response manner. Patients with conditions such as dry-eye disease and other chronic corneal conditions could be greatly benefited by such properties.

That significant (and similar) amounts of the CB2RA Composition were found in the posterior segment after only topical application supports the ability of the CB2RA Formulation to provide effective treatment of back of eye, retinal conditions, such as Age-Related Macular Degeneration (AMD) or Diabetic Retinopathy (DR) using a simple eye drop rather than the intravitreal administration of drugs required presently. Such an advance would greatly benefit treatment of these blinding diseases.

### Example 30. Degradation Products and Strategies for their Control

TA-A001 (aka the CB2RA composition) is a synthetic small molecule, new molecular entity comprising the well characterized CB2R agonist, Hu-308, along with a small, but controlled amount of its enantiomeric analogue, Hu-433. The general structure and composition of the CB2RA composition along with the chemical names of its two enantiomers is shown below:

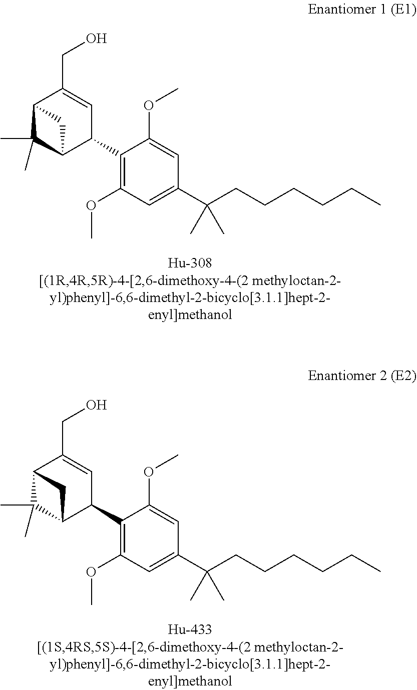

The CB2RA composition has been shown to possess potent anti-inflammatory and analgesic properties in a number of acute pain models when administered as an ophthalmic instillation post ocular burn, or intravenously post incisional surgery (ibid) and it possesses great potential value as a therapeutic agent for mammalian use in these and other therapeutic settings. TA-A001 has also been shown to be antiangiogenic.

Forced Degradation Studies

To qualify for use in humans the CB2RA composition must undergo rigorous analysis in terms of its efficacy and safety. Safety testing involves knowledge and understanding of its molecular chemistry and its degradation products and potential metabolites.

The potential for CB2RA composition degradation, and any new species formed when the CB2RA composition degrades may be assessed and determined by undertaking forced degradation studies. Forced degradation studies (FDS) involve exposing an active agent such as the CB2RA composition to environmentally harsh conditions; the conditions used for FDS undertaken with the CB2RA composition are shown in Table 33.

TABLE 33
Conditions to which TA-A001 was exposed
during forced degradation studies.

| Test | Reagent | Test Conditions |
|---|---|---|
| Acid hydrolysis | Hydrochloric acid | Room temperature, 6 hours |
| | (1M) | |
| Base hydrolysis | Sodium hydroxide | Room temperature, 8 hours |
| | (1M) | |
| Oxidation | Hydrogen peroxide | Room temperature, 24 hours |
| | (0.5M) | |
| Exposure to UV | 365 nm UV light | Room temperature, 5 days |
| Light | | |

After exposure to the above conditions, samples of the CB2RA composition were subject to reverse phase chromatography (Analytical Method 1) employing the conditions described in Table 34.

TABLE 34
Chromatographic conditions Analytical Method 1

| System | Agilent 1100 HPLC |
|---|---|
| Column | Zorbax-Eclipse XDB-C8 5 μm, 15 × 4.6 mm |
| | column (Agilent Technologies) |
| Column Temperature (° C.) | 30 |
| Solvent Conditions | Mobile phase: water/acetonitrile |
| | Gradient (time (min)/% acetonitrile): |
| | 0/50, 20/100, 30/100, 40/50 |
| Flow Rate | 1.0 mL/min |
| Detection | 210 nm |
| Injection Volume (μL) | 10 |

The number of newly generated species under all stated conditions, along with their relative retention times (RRT) with respect to the parent CB2RA composition peak (Peak 13: RRT=1) are provided in Table 35.

TABLE 35
Degradation products and relative retention
times (RRT) versus the parent TA-A001 peak.

| | | Percentage | Stress |
|---|---|---|---|
| Peak Number | RRT | Composition (%)* | Condition |
| 1 | 0.34 | 0.73 | Oxidation |
| 2 | 0.46 | 0.36 | UV Light |
| 3 | 0.48 | 0.45 | UV Light |
| 4 | 0.49 | 0.40 | UV Light |
| 5 | 0.50 | 0.30 | Oxidation |
| 6 | 0.53 | 0.86 | UV Light |
| 7 | 0.56 | 0.63 | UV Light |
| 8 | 0.58 | 0.99 | UV Light |
| 9 | 0.64 | 0.25 | Oxidation |
| 10 | 0.70 | 0.57 | Acid |
| 11 | 0.83 | 0.32 | Oxidation |
| 12 | 0.96 | 2.81 | Oxidation |
| 13 (Parent) | 1 | NA | NA |
| 14/15 | 1.21 | 28.83/0.61 | Acid/Alkali |

*Area under the curve for the peak with respect to area under the curve for the non-treated parent TA-A001 peak

Formulation of TA-A001

The CB2RA composition is a highly insoluble compound with an aqueous solubility of less than 0.5 mg/L. To overcome this barrier to delivery for pharmaceutical use the CB2RA composition may be formulated using water soluble PVP-PLA block copolymers to increase its apparent solubility. Thus, PVP-PLA block copolymers when above their critical micelle concentration (CMC) form unimodal micelles of approximately 30 nm diameter. During micelle formation the majority of the CB2RA composition becomes entrapped in the core of the micelle (bound drug) with a portion of compound remaining in aqueous solution below its solubility limit (free drug). When administered to mammals, after which free drug is absorbed, bound drug is released from the core to maintain the free drug/bound drug equilibrium. PVP-PLA micelles therefore represent a drug depot for efficient delivery of high concentrations of insoluble drugs. One such formulation, formulation (the CB2RA composition formulation 1) is described in Table 35. It can be used, among other routes, for topical, ocular, parenteral, oral, intranasal, pulmonary, rectal or intracerebral delivery of the CB2RA composition. The formulation was prepared as follows;

In a first step PVP-PLA block copolymer (0.539 g) was dissolved in dry ethanol (6.78 g) with magnetic stirring for 10 minutes at room temperature. 11 mg of the CB2RA composition was then added with stirring followed by drop wise addition of water (3.67 g). The resulting clear solution was left under stirring for 10 minutes at room temperature.

Ethanol was then removed and the PVP-PLA: CB2RA composition concentrated to 10% of its initial weight using a Rocket Synergy evaporator (ThermoFisher Scientific) set in HPLC fraction mode for 150 minutes. Phosphate buffer (600 mM, 0.37 mL) was then added to the concentrated solution followed by the addition water to obtain final concentration of the CB2RA composition 4 g/L. One millilitre of the bulk solution was then transferred by pipette into 10 mL glass vials. Vials were then lyophilized using a VirTis Genesis 25EL lyophilizer. The composition of the resulting lyophilized cakes is shown in Table 35.

TABLE 35
Composition of the CBN2RA composition formulation 1

| | | Amount/Vial by |
|---|---|---|
| Component | mg/vial | Weight (%) |
| CB2RA Composition | 4.0 | 1.9% |
| PVP-PLA copolymer | 196.0 | 92.9% |
| Sodium hydrogen phosphate | 9.7 | 4.6% |
| Sodium dihydrogen phosphate | 1.4 | 0.6% |
| Total | 211.1 | 100% |

Other formulations comprising PVP-PLA block copolymers and the CB2RA composition may be envisaged by those skilled in the art.

Stability Studies with the CB2RA Composition Formulation

It is important when developing a new formulation for therapeutic use in mammals that the stability of the drug substance in the formulation (in this case the CB2RA composition) is known so that a shelf-life for the formulation may be ascribed. These studies are performed under various conditions both at room temperature and at elevated temperatures including temperatures of 40° C., so called accelerated conditions. CB2RA composition formulation 1 (Table 35) was subject to stability assessment in glass vials under accelerated conditions, the degradation profile of the CB2RA composition being monitored using Analytical Method 1 as herein described. When studying the degradation of the CB2RA composition under these conditions a new degradation product, with an RRT of 1.08 was observed which had not been seen when studying the CB2RA composition alone in the presence of UV light, strong acid, alkali, or oxidizing agent. Unlike the other degradation products observed in forced degradation studies, amounts of this new compound with an RRT of 1.08, (henceforth termed Compound B) rose rapidly over time to levels exceeding those set conventionally for a pharmaceutical product; amounts of Compound B formed over time during storage of the CB2RA composition formulation 1 under accelerated conditions are shown in Table 36.

TABLE 36
Amounts of compound B formed over 6 months storage of the
CB2RA composition formulation under accelerated conditions.

| | Percentage Composition |
|---|---|
| Month | (%) |
| 0 (pre-study) | 0 |
| 1 | 1.1 |
| 2 | 2.3 |
| 6 | 4.7 |

Compound B was therefore the predominant degradation product of the CB2RA composition formulation 1 albeit Compound B was not formed when the CB2RA composition alone was subject to forced degradation studies using the conditions described in Table 33. Forced degradation studies where the CB2RA composition alone was exposed to heat (80° C.) only, for 24 hr and 48 hr were then performed to determine whether Compound B could be formed by the CB2RA composition alone; results are presented in Table 6. As may be seen, heating the CB2RA composition alone under these conditions generated Compound B along with a number of new degradation products of lesser concentration, that were not seen after exposure of the CB2RA composition to acid, alkali, or peroxide. The peaks shown in Tables 34 and 37 therefore represent new CB2RA compositions.

TABLE 37
New degradation products formed by
high temperature forced degradation

| | |
|---|---|
| 24 hr | 48 hr |

| | | Percentage | | Percentage |
|---|---|---|---|---|
| | | Composition | | Composition |
| Peak Number | RRT | (%) | RRT | (%) |
| 1 | 0.80 | 0.40 | 0.80 | 0.53 |
| 2 | 0.85 | 0.27 | 0.85 | 0.28 |
| 3 | 0.94 | 0.88 | 0.94 | 1.22 |
| 4 | 1.00 | 84.4 | 1.00 | 67.5 |
| 5 | 1.03 | 0.34 | 1.03 | 0 |
| 6 | 1.08 | 11.6 | 1.08 | 13.5 |
| 7 | 1.11 | 0.35 | 1.11 | 0.43 |

As Compound B is the major degradation product of the CB2RA composition it was important to determine its structure and method of formation. Amounts of the CB2RA composition subject to 24 hr exposure to dry heat were therefore processed by semi-preparative reverse phase UV/MS chromatography using the conditions shown in Table 38.

TABLE 38
Chromatographic conditions used for semi-preparative
reverse phase chromatography (Analytical Method 2)

| System | Waters ARC Semi-Preparative HPLC |
|---|---|
| | with 3100 Mass Detector |
| Column | Sunfire C8, 19 × 100mm, 5 μm |
| Column Temperature (° C.) | 30 |
| Solvent Conditions | Mobile phase: water/acetonitrile |
| | Gradient (time (min)/% acetonitrile): |
| | 0/55, 1/55, 20/90, |
| Flow Rate | 1.0 mL/min |
| Detection | UV: 200-400 nm. MS: SIM: m/z 413 |
| Injection Volume (μL) | 10 |

Amounts of Compound B were collected by this method the structure of the new molecule then being determined by solution NMR (1H 1D, 13C 1D, 1H-1H COSY, 1H-13C HSQC and 1H-13C HMBC) using a Bruker AVANCE II 700 MHz spectrometer fitted with a Bruker 5 mm C/H cryoprobe. Samples were dissolved in deuterated chloroform prior to analysis which was performed at a temperature of 298K. The structure of Compound B determined by this method is shown in the schematic below with the site of oxidation circled:

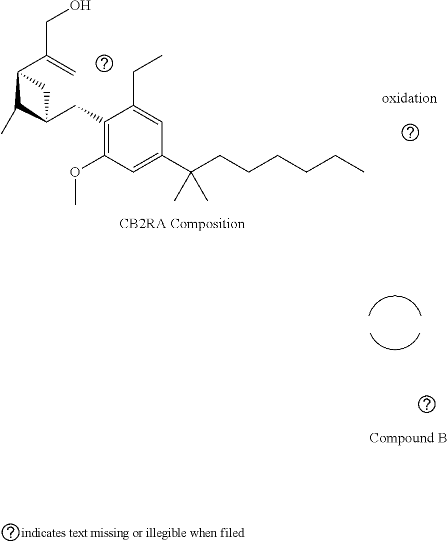

Compound B as formed by dry heating of the CB2RA composition has not been described before in the literature and represents a new molecular entity. Analysis of its structure suggests it is formed by oxidation of the single hydroxyl in the molecule by a novel process. That Compound B is formed by oxidation is surprising since exposure to the strongly oxidizing conditions generated by hydrogen peroxide failed to produce this degradation product. Compound B is therefore a new molecular entity formed by process as yet undisclosed in the literature. Analysis of Compound B using chiral chromatography demonstrated that Compound B was itself enantiomeric with an enantiomer ratio equal to that of the CB2RA composition from which it was formed; both enantiomers of the CB2RA composition undergo degradation to Compound B at the same rate.

Controlling the Production of Compound B in the CB2RA Composition

It is important for any pharmaceutical product that impurities including degradation products are kept within certain limits as such impurities may prove toxic to mammals exposed to the pharmaceutical product. Alternatively, it can be important to control the amount of impurities even when they contribute desirable activities. Methods and processes to control levels of impurities, in the case of the CB2RA composition, most importantly Compound B, the major degradation product, are therefore highly desirable. However, given the unexpected and unproven mechanism apparently causing the formation of Compound B it was not initially clear which methods would be successful to control its production. A number of different approaches to inhibiting Compound B formation were evaluated:

Studies with the CB2RA Composition Alone

As the putative process of formation was oxidation initial experiments sought to determine whether removal of oxygen from the incubation conditions would change the amount of Compound B generated by the CB2RA composition. Here samples of the CB2RA composition alone were incubated for 24 hr and 48 hr at elevated temperature in capped glass vials either in the presence of laboratory air or in vials flushed with inert gas (argon or nitrogen). Results are shown in Table 40.

TABLE 40
Effect of removal of oxygen from the incubation conditions

| Incubation | Composition Compound B (%) | |
|---|---|---|
| Conditions | 24 hr | 48 hr |
| 24 hr @ 65° C. (Laboratory Air) | 5.6 | 10.4 |
| 24 hr @ 65° C. (Inert Gas) | 0.42 | 0.59 |
| Percentage Reduction Compound | 92.5% | 94.3% |
| B by adding Nitrogen | | |

As is clear, while heating to 65° C. for extended periods generated Compound B directly from the CB2RA composition, incubation under inert gas reduced levels of Compound B formed by heating in air by over 90% over 48 hr.

Studies with the CB2RA Composition Formulation

Effect of Inert Gases

Although incubation with inert gases inhibited formation of Compound B from the CB2RA composition when the composition was tested in isolation it was not clear whether this would be the case when the CB2RA composition was incubated as part of CB2RA composition formulation 1 where excipients are included. Vials of CB2RA composition formulation 1 were therefore prepared and dried as described herein and at the end of the lyophilization process were either back-filled with nitrogen prior to capping or back-filled with laboratory air prior to capping. The two CB2RA composition formulation 1 preparations were then subjected to the test program described in Table 41.

TABLE 41
Effect of inert gas on the formation of Compound
B in CB2RA composition formulation 1.

| | Composition |
|---|---|
| | Compound B |
| Incubation | (%) |
| Conditions | 24 hr |
| 24 hr @ 65° C. (Laboratory Air) | 0.53 |
| 24 hr @ 65° C. (Nitrogen Purge) | 0.07 |
| 1 Month @ 40° C. (Nitrogen Purge) | 0 |

As may be seen, purging with inert gas effectively inhibited production of Compound B in the formulation for at least one month.

Effect of Oxidation Inhibitors (Antioxidants)

While it is clear that removal of oxygen after drying of CB2RA composition formulation 1 inhibited Compound B for at least one month at accelerated temperatures, it was not known whether this procedure would provide adequate protection for the formulation over longer periods. The effect of adding antioxidants to the formulation prior to drying was therefore assessed. As the exact mechanism by which oxidation occurs was not obvious (viz. oxygen causes Compound B formation while hydrogen peroxide does not) a number of different antioxidants were evaluated as listed in Table 42.

TABLE 42
Example antioxidants for control of Compound B production

| | Mechanism of | |
|---|---|---|
| Antioxidant Name | Inhibition | Comment |
| Cysteine hydrochloride | Reactive oxygen | Directly removes oxygen |
| Vitamin E (alpha- | species scavengers | free radicals that induce |
| tocopherol) | | ketone formation |
| Butylated hydroxyanisol | | |
| (BHA) | | |
| Sodium bisulphite | Free oxygen | Removes oxygen by |
| Sodium metabisulphite | reactants | chemical reaction with |
| | | oxygen |
| Sorbitol | Metal ion chelators | Remove metal ions that |
| EDTA | | catalyze oxygen radical |
| | | production |

All were used at concentrations acceptable for pharmaceutical formulation by reference amounts used in parenteral and ophthalmic products approved for human used, Example CB2RA composition formulations prepared in the same manner as CB2RA composition formulation 1 including the antioxidant are provided in Tables 43-49 below.

The effect of the various antioxidants on the formation of Compound B was assessed by including them in the following example formulations:

CB2RA Composition Formulation Comprising Cysteine

PVP-PLA block copolymer (0.541 g) was dissolved in ethanol (6.78 g) with magnetic stirring for 10 minutes. To this mixture was added the CB2RA composition followed by drop wise addition of water (3.08 mL) and then addition of 0.58 mL of 1 mg/mL of cysteine solution. The resulting clear solution was left under stirring for 10 minutes at room temperature and then processed identically to CB2RA composition formulation 1. The quantitative composition of each vial is shown in Table 43.

TABLE 43
CB2RA composition formulation comprising cysteine.

| | | Amount/Vial by |
|---|---|---|
| Ingredients | mg/vial | Weight (%) |
| Composition containing E1 & | 4.0 | 1.9% |
| E2 | | |
| PVP-PLA copolymer | 196.0 | 92.8% |
| Sodium hydrogen phosphate | 9.7 | 5 |
| | | 4.6% |
| Sodium dihydrogen phosphate | 1.4 | 0.7% |
| Cysteine•HCl | 0.2 | 0.1% |
| Total | 211.3 | 100% |

CB2RA Composition Formulation Comprising Vitamin E

PVP-PLA block copolymer (0.541 g) was dissolved in ethanol (6.78 g) with magnetic stirring for 10 minutes. Next To this mixture was added the CB2RA composition followed by drop wise addition of water (3.08 mL) and then 1.16 mL of a 10 mg/mL ethanolic solution of vitamin E succinate. The resulting clear solution was left under stirring for 10 minutes at room temperature and then processed identically to CB2RA composition formulation 1. The quantitative composition of each vial is shown in Table 44.

TABLE 44
CB2RA composition formulation comprising vitamin E

| | | Amount/Vial by |
|---|---|---|
| Ingredients | mg/vial | Weight (%) |
| Composition containing E1 & | 4.0 | 1.9% |
| E2 | | |
| PVP-PLA copolymer | 196.0 | 92.6% |
| Sodium hydrogen phosphate | 9.7 | 4.6% |
| Sodium dihydrogen phosphate | 1.4 | 0.7% |
| Vitamin E succinate | 0.4 | 0.2% |
| Total | 211.5 | 100% |

CB2RA Composition Formulation Comprising BHA.

PVP-PLA block copolymer (0.541 g) was dissolved in ethanol (5.86 g) with magnetic stirring for 10 minutes. Next, 1.16 mL of ethanolic BHA solution (1 mg/mL) was added to the mixture. 11 mg of the CB2RA composition was then added followed by drop wise addition of water (3.67 mL). The resulting clear solution was left under stirring for 10 minutes at room temperature and then processed identically to CB2RA composition formulation 1. The quantitative composition of each vial is shown in Table 45.

TABLE 45
CB2RA composition formulation comprising BHA.

| | | Amount/Vial by |
|---|---|---|
| Ingredients | mg/vial | Weight (%) |
| Composition containing E1 & | 4.0 | 5 |
| E2 | | 1.9% |
| PVP-PLA copolymer | 196.0 | 92.6% |
| Sodium hydrogen phosphate | 9.7 | 4.6% |
| Sodium dihydrogen phosphate | 1.4 | 0.7% |
| BHA | 0.4 | 0.2% |
| Total | 211.5 | 100% |

CB2RA Composition Formulation Comprising Sodium Bisulphite.

PVP-PLA block copolymer (0.541 g) was dissolved in ethanol (6.77 g) with magnetic stirring for 10 minutes. 11 mg of the CB2RA composition was then added followed by drop wise addition of sodium bisulfite solution in water (3.23 mg/mL, 3.66 mL). The resulting clear solution was left under stirring for 10 minutes at room temperature after which it was processed identically to CB2RA composition formulation 1. The quantitative composition of each vial is shown in Table 46.

TABLE 46
CB2RA composition formulation comprising sodium bisulphite

| | | Amount/Vial by |
|---|---|---|
| Ingredients | mg/vial | Weight (%) |
| Composition containing E1 & E2 | 4.0 | 1.9% |
| PVP-PLA copolymer | 196.0 | 91.0% |
| Sodium hydrogen phosphate | 9.7 | 4.5% |
| Sodium dihydrogen phosphate | 1.4 | 0.6% |
| Sodium bisulfite | 4.3 | 2.0% |
| Total | 215.4 | 100% |

CB2RA Composition Formulation Comprising Sodium Metabisulphite.

PVP-PLA block copolymer (0.541 g) was dissolved in ethanol (6.77 g) with magnetic stirring for 10 minutes. 11 mg of the CB2RA composition was then added followed by drop wise addition of sodium metabisulfite solution in water (3.23 mg/mL, 3.66 mL). The resulting clear solution was left under stirring for 10 minutes at room temperature after which it was processed identically to CB2RA composition formulation 1. The quantitative composition of each vial is shown in Table 47.

TABLE 47
CB2RA composition formulation comprising
sodium metabisulphite

| | | Amount/Vial by |
|---|---|---|
| Ingredients | mg/vial | Weight (%) |
| Composition containing E1 & E2 | 4.0 | 1.9% |
| PVP-PLA copolymer | 196.0 | 91.0% |
| Sodium hydrogen phosphate | 9.7 | 4.5% |
| Sodium dihydrogen phosphate | 1.4 | 0.6% |
| Sodium metabisulfite | 4.3 | 2.0% |
| Total | 215.4 | 100% |

CB2RA Composition Formulation Comprising Sorbitol

PVP-PLA block copolymer (0.541 g) was dissolved in ethanol (6.74 g) with magnetic stirring for 10 minutes. 11 mg of CB2RA composition was then added followed by drop wise addition of aqueous sorbitol solution (17.7 mg/mL, 3.64 mL). The resulting clear solution was left under stirring for 10 minutes at room temperature after which it was processed identically to CB2RA composition formulation 1. The quantitative composition of each vial is shown in Table 48.

TABLE 48
CB2RA composition formulation comprising sorbitol

| | | Amount/Vial by |
|---|---|---|
| Ingredients | mg/vial | Weight (%) |
| Composition containing E1 & E2 | 4.0 | 1.7% |
| PVP-PLA copolymer | 196.0 | 83.6% |
| Sodium hydrogen phosphate | 9.7 | 4.1% |
| Sodium dihydrogen phosphate | 1.4 | 0.6% |
| Sorbitol | 23.4 | 10.0% |
| Total | 234.5 | 100% |

CB2RA Composition Formulation Comprising Sodium EDTA.

PVP-PLA block copolymer (0.541 g) was dissolved in ethanol (6.74 g) with magnetic stirring for 10 minutes. 11 mg of CB2RA composition was then added followed by drop wise addition of aqueous EDTA solution (10.0 mg/mL, 2.9 mL). The resulting clear solution was left under stirring for 10 minutes at room temperature after which it was processed identically to CB2RA composition formulation 1. The quantitative composition of each vial is shown in Table 49.

TABLE 49
CB2RA composition formulation comprising EDTA

| | | Amount/Vial by |
|---|---|---|
| Ingredients | mg/vial | Weight (%) |
| Composition containing E1 & E2 | 4.0 | 1.9% |
| PVP-PLA copolymer | 196.0 | 92.4% |
| Sodium hydrogen phosphate | 9.7 | 4.5% |
| Sodium dihydrogen phosphate | 1.4 | 0.7% |
| EDTA | 1.1 | 0.5% |
| Total | 211.9 | 100% |

Forced Degradation Studies Performed with the Antioxidant Containing Formulations.

Capped vials from each of the cysteine hydrochloride, vitamin E, butylated hydroxyanisol (BHA), sodium bisulphite, sodium metabisulphite, sorbitol and EDTA formulation were then subject to FDS by heating at 65° C. containing either laboratory air or backfilled with nitrogen. Results of these studies are shown in Table 50.

TABLE 50
Relative Effectiveness of the various antioxidants assessed

| | Increase in Compound B | | | |
|---|---|---|---|---|
| | composition within the | | | |
| | formulation (%) | | Relative | |
| | Laboratory | | Effectiveness | |
| Antioxidant | Air (A) | Nitrogen (B) | (A + B)/2 | Ranking |
| Cysteine | 0.45 | 0.23 | 0.565 | 3 |
| hydrochloride | | | | |
| Vitamin E (alpha- | 0.64 | 0.08 | 0.68 | 4 |
| tocopherol) | | | | |
| Butylated | 0.20 | 0.04 | 0.22 | 1 |
| hydroxyanisol | | | | |
| Sodium bisulphite | 0.69 | 0.17 | 0.775 | 5 |
| Sodium | 0.91 | 0.2 | 1.01 | 7 |
| metabisulphite | | | | |
| Sorbitol | 0.35 | 0.06 | 0.38 | 2 |
| EDTA | 0.75 | 0.12 | 0.81 | 6 |

As Table 50 shows while 6 out of the seven antioxidants assessed were effective, to various extents, in hindering Compound B formation, sodium metabisulphite caused an increase in the amount of the impurity after 24 hr of incubation.

### SUMMARY

Herein are described new CB2RA compositions, comprising unchanged relative amounts of the two enantiomeric starting components, that are formed during storage of the CB2RA composition under various conditions, that are CB2RA composition degradation products. These new species may be formed in vitro and in vivo either due to body temperature or enzymic or other metabolic processes. Most importantly a new, as yet undescribed degradation product has been discovered that forms in amounts greater than those of other degradation products to levels exceeding those permitted in pharmaceutical products. The new species (Compound B) is described in detail and also the mechanism by which it forms. As stated, the chemical mechanism of Compound B formation (oxidation) was unexpected because the molecule was not generated by the strongly oxidizing conditions imparted by hydrogen peroxide. The structure of Compound B was therefore equally unexpected given the resistance of the CB2RA composition to hydrogen peroxide. Certain ratios of CB2RA composition/Compound B are described that equally could not have been predicted but which may be useful when using the CB2RA composition for therapeutic purposes. In summary, initial experiments taught away from oxidation as the method Compound B generation so that skilled experimentalists would not have expected oxidation to be the route of synthesis had they obtained the same results. It was only through stability studies with the CB2RA formulation that this new degradant was identified.

As Compound B is an impurity its concentration should be controlled. Herein are described a number of methods for controlling its generation. Given the unexpected mechanism of Compound B production these methods could not have been predicted to be successful a priori however all will inhibit oxidation of the single hydroxyl to form the ketone of FIG. 2. Other antioxidants that may be of value in inhibiting formation of Compound 2 can be used based on these findings and, along with those tested here, include ascorbic acid, butylated hydroxytoluene, sesamol, guaiac resin, methionine, citric acid, tartaric acid, phosphoric acid, thiol derivatives, potassium metabisulphite, ascorbyl palmitate, calcium stearate, propyl gallate, sodium thiosulphate, glutathione, dihydroxybenzoic acid, benzoic acid, urate and uric acid, sorbic acid, sodium benzoate and the like and mixtures thereof. Inert gases including nitrogen and argon are also useful alone and together with the chemical antioxidants.

Also of note is that while some antioxidants were effective at controlling the production of Compound B, others were not, those that were successful and those not, not falling into any one particular mechanism of action. Thus, it was not obvious a priori which antioxidants would be valuable and also not obvious therefore what concentration of antioxidant would be effective. Herein, some preferred antioxidants that may be used alone and in combination with each other and with inert gases. As stated herein, a range of CB2RA/Compound B compositions comprising from less 0.1% wt/wt Compound B to approximately 14% wt/wt compound B which may be of use therapeutically, and methods for generating these ratios and controlling the levels of Compound B to within conventional pharmaceutical limits.

### REFERENCES

Janeway, et al. (2001) IMMUNOBIOLOGY: THE IMMUNE SYSTEM IN HEALTH AND DISEASE. 5th edition. New York: Garland Science.
Krystel-Whittemore, et al. (2016) FRONT. IMMUNOL., 6: 620.
Zhang, et al. (2007) INTERNATIONAL ANESTHESIOLOGY CLINIC, 45(2): 27.
Vance, et al. (2009) CELL HOST & MICROBE, 6(1): 10.
Cadieux, et al. (2005) J. CELL SCIENCE, 118(Pt 7): 1437.
Zeilhofer, et al. (2007) BIOCHEMICAL PHARMACOL., 73: 165.
Funk (2001) SCIENCE, 294(5548): 1871.
Reinold et al. (2005) J. CLIN. INVEST., 115: 673.
Lehnardt et al. (2003) PROC. NATL. ACAD. SCI. USA, 100: 8514.
Ricciotti, et al. (2011) ARTERIOSCLER. THROMB. VASC. BIOL., 31(5): 986.
Ferreira S H, et al. (1988) NATURE, 334: 698.
Zelenka, et al. (2005) PAIN, 116(3): 257.
Hess, et al. (2011) PROC. NATL. ACAD. SCI. USA, 108(9): 3731.
Teng, et al. (2017) J. IMMUNOL RES., 2017: 9671604.
Brinkmann, et al. (2004) SCIENCE, 303(5663): 1532.
Wang (2018) CELL TISSUE RES., 371(3): 531.
Garn, et al. (2016) J. ALLERGY AND CLIN. IMMUNOL., 138(1): 47.
Voscopoulos, et al. (2010) BR. J. ANAESTH, 105(S1): 69.
Hoffman, et al. (2016) J. CATARACT REFRACT. SURG., 42(9): 1368.
Razmju, et al. (2012) INT. J. PREV. MED., 3(Supp 1): S199.
Sameen, et al. (2017) PAK. J. MED. SCI., 33(5): 1101.
Colligris, et al. (2014) SAUDI J. OPHTHALMOL. 28(1): 19.
Iwamoto, et al. (2017) NATURE SCIENTIFIC REPORTS, 7:13267.
Flach (2001) TRANS. AM. OPHTHALMOL. SOC., 99: 205.
Arfè, et al. (2016) BRIT. MED. J., 354: i4857.
Kompella, et al. (2010) EXPERT OPIN. DRUG DELIV., 7(5): 631.
Highlights of Prescribing information for CELEBREX® June 2018 revision available at www.accessdata.fda.gov/drugsatfda_docs/label/2018/020998s0501bl.pdf
Oren, et al. (2004) BIOPHYS. J. 87(2): 768.
Hua (2013) FRONT PHARMACOL., 4:127.
Dostert, et al. (2004) CURR. PHARM. DES., 10: 2807.
Couper, et al. (2008) J. IMMUNOL., 180(9): 5771.
Dennison, et al. (1998) B.M.J., 316(7134): 789.
Mohammad, et al., (2017) SYSTEMATIC REV., 6(1): 92.
Motov, et al. (2017) ANN. EMERG. MED., 70(2): 177.
Williams (2018) CLIN. PHARMACOL. CORTICOSTEROIDS RESP. CARE, 63(6): 655.
Becker (2013) ANESTHESIA PROGRESS, 60(1): 25.
Weiser, et al. (2016) BULL. WORLD HEALTH ORG., 94(3): 201.
Gan, et al. (2014) CURR. MED. RES. OPIN., 30(1): 149.
Gan (2017) J. PAIN RES. 10: 2287.
Kehlet, et al. (2001) BR. J. ANAESTH. 87(1): 62.
Zhao, et al. (2019) PAIN RES. MANAGE., 2019: 7490801.
Lail et al. (2014) CAN. J. HOSP. PHARM., 67(5): 337.
Garimella (2013) CLINICS COLON RECTAL SURG., 26(3): 191.
Wunsch, et al. (2016) JAMA 315(15): 1654.
Galor et al. (2015) EYE, 29: 301.
Pereira et al. (2017) PAIN PHYSICIAN, 20: 429.
Trattler et al. (2017) CLIN. OPHTHALMOL., 11: 1423.
Shtein (2011) EXPERT REVIEW OPHTHALMOL., 6(5): 575.
Schwatrtz (2018) JAMA INTERN. MED., 178(2): 181.
Dana et al. (2019) AM. J. OPHTHALMOL. 202: 47.
Ong et al. (2007) CLIN. MED. RES., 5(1):19.
Comprehensive Organic Chemistry. B. M. Trost & I. Fleming, eds., 1991-1992.
Martin, Remington's Pharmaceutical Sciences, 15th Ed., Mack Publ. Co., Easton, P A, 1975.
Soethoudt et al. (2017) NATURE COMMUN., 8: 13958.
Morales et al. (2017) FRONT. PHARMACOL., 8: 422.
Smoum et al. (2015) PROC. NATL. ACAD. SCI. USA, 112.28: 8774.
Patent No. WO2018176158A1.
Lengyel, et al. (2019) SCIENTIA PHARMACEUTICA, 87(3): 20.
Jainyu (2016) NAT. REV. MATER., 1(12): 1.
Canadian Patent No. 3034722.
Yang et al. (2018) YALE J. BIOL. MED., 91(1): 13.
Shtein (2011) EXPERT REV. OPHTHALMOL., 6(5): 575.
Javadi, Feizi (2011) J. OPHTHALMIC VIS. RES., 6(3): 192.
Ozan-Yuksel et al. (2009) EXPER. EYE RES., 88(4): 769.
Kauppinen, et al. (2016) CELL. MOL. LIFE SCI., 73(9): 1765.
Kashiwagi et al. (2003) BR. J. OPHTHALMOL. 87297.
Sharma et al. (2019) METHODS IN MICROBIOL. 46: 93.
Labuda et al. (2005) EUROPEAN J. OF PHARMACOL., 527: 172.
Rimon et al., (2010) PROC NATL ACAD SCI USAUSA, 107(1):28.
https://www.reviewofoptometry.com/article/topical-nsaid-update
Mohammadpour et al. (2018) J. CURR. OPHTHALMOL., 30(2): 110.
Iwamoto et al. (2017) SCI REP 7: 13267.
Baudouin et al. (2018). ACTA OPHTHALMOL., 96(2): 111.
Yamaguchi (2018) INVEST. OPTHALMOL. VIS. SCI., 59(14): DES192.
Hessen, Akpek (2014) J OPHTHALMIC VIS RES., 9(2): 240.
Chung et al. (2019) J DIABETES RES. 2019: 8164250.
Thapa et al. (2018) CANNABIS CANNABINOID RES., 3(1): 11.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A composition for agonizing CB2 receptor activity in a subject, the composition comprising a combination of:
(a) a first compound of Formula I: 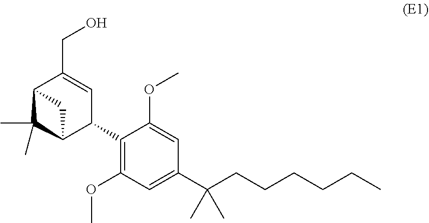 [SMILES: CCCCCCC(C)(C)c1cc(OC)c([C@H]2C=C(CO)[C@H]3C[C@@H]2C3(C)C)c(OC)c1]
or a pharmaceutically acceptable salt thereof; and
b. a second compound of Formula II: 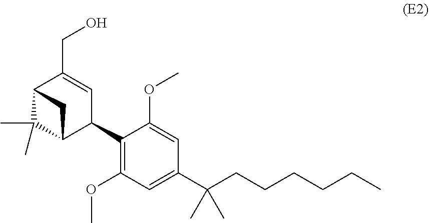 [SMILES: CCCCCCC(C)(C)c1cc(OC)c([C@@H]2C=C(CO)[C@@H]3C[C@H]2C3(C)C)c(OC)c1]
or a pharmaceutically acceptable salt thereof.

2. The composition of claim 1, wherein the composition comprises the compound of Formula I and the compound of Formula II in a weight ratio of from 99.85:0.15 to 93.5:6.5, and a pharmaceutically acceptable excipient.

3. The composition of claim 2, wherein the weight ratio of the compound of Formula I to the compound of Formula II is from 99.8:0.2 to 98.2:1.8.

4. The composition of claim 2, wherein the weight ratio of the compound of Formula I to the compound of Formula II is from 98.8:1.2 to 98.4:1.6.

5. The composition of claim 4, wherein the weight ratio of the compound of Formula I to the compound of Formula II is from 99.3:0.7 to 98.7:1.3.

6. The composition of claim 5, wherein the weight ratio of the compound of Formula I to the compound of Formula II is about 99:1.

7. The composition of claim 1, wherein the pharmaceutically acceptable excipient comprises a polymer, a solubilizing agent, a buffer, a salt, a preservative or a combination thereof.

8. The composition of claim 7, wherein the polymer is a polyvinylpyrrolidone-polylactic acid (PVP-PLA) copolymer.

9. The composition of claim 8, wherein the PVP-PLA copolymer has the structure of Formula III: 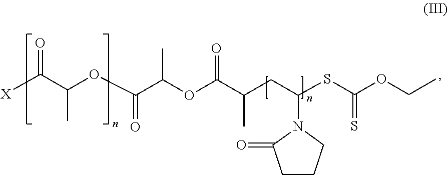
wherein X is an initiator alcohol having a boiling point greater than 145° C., n is, on average, from 20 and 40, and m is, on average, from 10 and 40, wherein the block copolymers have a number average molecular weight (Mn) of at least 3,000 Da.

10. The composition of claim 7, wherein the buffer is a phosphate buffer.

11. The composition of claim 7, wherein the salt is a sodium salt, or a potassium salt.

12. The composition of claim 7, wherein the composition is in the form of a micellar preparation.

13. The composition of claim 12, wherein the micellar preparation is in the form of a liquid.

14. The composition of claim 12, wherein the micellar preparation is dehydrated into a solid form.

15. The composition of claim 14, wherein the composition comprises from about 0.25% w/w to about 60% w/w, from about 0.5% w/w to about 40% w/w, from about 0.75% w/w to about 30%, or about 1% w/w to about 20% w/w of the first and second compounds in combination.

16. The composition of claim 7, wherein the composition comprises from about 5% to about 95%, or from 30% to about 90%, from about 60% to about 85%, or from about 70% to about 80%, by weight of the polymer.

17. The composition of claim 7, wherein the composition comprises from about 1% to about 20% by weight the buffer.

18. The composition of claim 7, wherein the composition further comprises an emulsifying agent, an antioxidant, a controlled release agent, a lubricant, or a flavoring agent.

19. The composition of claim 13, wherein the solid form has been rehydrated in a solvent to produce a micellar solution.

20. The composition of claim 19, wherein the solvent is water, an alcohol, a dextrose solution, or saline.

21. The composition of claim 19, wherein the concentration of the first and second compounds in combination is from about 0.5 mg/mL to about 15 mg/mL.

22. The composition of claim 19, wherein the composition has a pH from about 5 to about 9, from about 6 to about 8, or from about 6.5 to about 7.5 and/or has a viscosity in the range from about from about 0.2 mPas to about 80,000 mPas.

23. The composition of claim 21, wherein the micellar solution comprises particles having a particle size (Z.av) of 12-50 nm, 15-45 nm, or 20-40 nm.

24. The composition of claim 21, wherein the particles have a polydispersity index (PDi) of from about 0.05 to about 0.15.

25. The composition of claim 1, wherein the composition further comprises from 0.015% to 1.5% of a third or fourth compound.

26. The composition of claim 1, which comprises up to 15% wt/wt of an impurity which has a structure of Formula B:

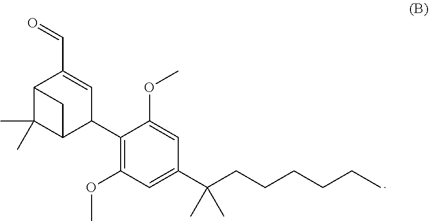 [SMILES: CCCCCCC(C)(C)c1cc(OC)c(C2C=C(C=O)C3CC2C3(C)C)c(OC)c1]

27. The composition of claim 26, which comprises up 13% wt/wt of the impurity, up to 10% wt/wt, up to 5% wt/wt, up to 2% wt/wt, up to 1% wt/wt, up to 0.1% wt/wt, up to 0.01% wt/wt, or up to 0.001% wt/wt of the impurity.

28. The composition of claim 26, which further comprises an antioxidant for reducing formation of the impurity.

29. The composition of claim 28, wherein the antioxidant is selected from the group consisting of ascorbic acid, butylated hydroxytoluene, sesamol, guaiac resin, methionine, citric acid, tartaric acid, phosphoric acid, thiol derivatives, potassium metabisulphite, ascorbyl palmitate, calcium stearate, propyl gallate, sodium thiosulphate, glutathione, dihydroxybenzoic acid, benzoic acid, urate and uric acid, sorbic acid, sodium benzoate, EDTA, sodium bisulphite, vitamin E, cysteine hydrochloride, sorbitol, butylated hydroxyanisol, and mixtures thereof.

30. The composition of claim 28, wherein the antioxidant is selected from the group consisting of EDTA, sodium bisulphite, vitamin E, cysteine hydrochloride, sorbitol, butylated hydroxyanisol, and mixtures thereof.

31. The composition of claim 28, wherein the antioxidant comprises butylated hydroxyanisol.

32. The composition of claim 1, which is essentially free of oxygen.

33-96. (canceled)
